SEQUENCE LISTING (145 sequences)

SEQ ID NO: 1 - protein, 6 aa, synthetic construct
  REGION 1..6: note = peptide is circular
  SITE 2: note = L-2-naphthylalanine
FXRRRQ

SEQ ID NO: 2 - protein, 6 aa, synthetic construct
  REGION 1..6: note = peptide is circular
  SITE 2: note = L-2-naphthylalanine
FXRRRC

SEQ ID NO: 3 - protein, 6 aa, synthetic construct
  REGION 1..6: note = peptide is circular
  SITE 2: note = L-2-naphthylalanine
FXRRRU

SEQ ID NO: 4 - protein, 6 aa, synthetic construct
  REGION 1..6: note = peptide is circular
  SITE 4: note = L-2-naphthylalanine
RRRXFQ

SEQ ID NO: 5 - protein, 6 aa, synthetic construct
  REGION 1..6: note = peptide is circular
  SITE 5: note = L-2-naphthylalanine
RRRRXF

SEQ ID NO: 6 - protein, 6 aa, synthetic construct
  REGION 1..6: note = peptide is circular
  SITE 2: note = L-2-naphthylalanine
FXRRRR

SEQ ID NO: 7 - protein, 7 aa, synthetic construct
  REGION 1..7: note = peptide is circular
  SITE 2: note = D-2-naphthylalanine
  SITE 3: note = D-arginine
  SITE 5: note = D-arginine
  SITE 7: note = D-glutamine
FXRRRRQ

SEQ ID NO: 8 - protein, 7 aa, synthetic construct
  REGION 1..7: note = peptide is circular
  SITE 2: note = D-2-naphthylalanine
  SITE 3: note = D-arginine
  SITE 5: note = D-arginine
FXXRXRQ

SEQ ID NO: 9 - protein, 7 aa, synthetic construct
  REGION 1..7: note = peptide is circular
  SITE 2: note = L-2-naphthylalanine
FXRRRRQ

SEQ ID NO: 10 - protein, 7 aa, synthetic construct
  REGION 1..7: note = peptide is circular
  SITE 1: note = D-phenylalanine
  SITE 2: note = L-2-naphthylalanine
  SITE 4: note = D-arginine
  SITE 6: note = D-arginine
FXRRRRQ

SEQ ID NO: 11 - protein, 7 aa, synthetic construct
  REGION 1..7: note = peptide is circular
  SITE 5: note = L-2-naphthylalanine
RRFRXRQ

SEQ ID NO: 12 - protein, 7 aa, synthetic construct
  REGION 1..7: note = peptide is circular
  SITE 6: note = L-2-naphthylalanine
FRRRRXQ

SEQ ID NO: 13 - protein, 7 aa, synthetic construct
  REGION 1..7: note = peptide is circular
  SITE 1: note = D-arginine
  SITE 5: note = L-2-naphthylalanine
XRFRXRQ

SEQ ID NO: 14 - protein, 7 aa, synthetic construct
  REGION 1..7: note = peptide is circular
  SITE 3: note = L-2-naphthylalanine
RRXFRRQ

SEQ ID NO: 15 - protein, 8 aa, synthetic construct
  REGION 1..8: note = peptide is circular
CRRRRFWQ

SEQ ID NO: 16 - protein, 8 aa, synthetic construct
  REGION 1..8: note = peptide is circular
  SITE 2: note = D-phenylalanine
  SITE 3: note = L-2-naphthylalanine
  SITE 5: note = D-arginine
  SITE 7: note = D-arginine
FXXRXRXQ

SEQ ID NO: 17 - protein, 8 aa, synthetic construct
  REGION 1..8: note = peptide is circular
  SITE 3: note = L-2-naphthylalanine
FFXRRRRQ

SEQ ID NO: 18 - protein, 8 aa, synthetic construct
  REGION 1..8: note = peptide is circular
  SITE 6: note = L-2-naphthylalanine
RFRFRXRQ

SEQ ID NO: 19 - protein, 8 aa, synthetic construct
  REGION 1..8: note = peptide is circular
URRRRFWQ

SEQ ID NO: 20 - protein, 8 aa, synthetic construct
  REGION 1..8: note = peptide is circular
CRRRRFWQ

SEQ ID NO: 21 - protein, 8 aa, synthetic construct
  REGION 1..8: note = peptide is circular
  SITE 2: note = L-2-naphthylalanine
FXRRRRQK

SEQ ID NO: 22 - protein, 8 aa, synthetic construct
  REGION 1..8: note = peptide is circular
  SITE 2: note = L-2-naphthylalanine
FXRRRRQC

SEQ ID NO: 23 - protein, 8 aa, synthetic construct
  REGION 1..8: note = peptide is circular
  SITE 1: note = D-phenylalanine
  SITE 2: note = L-2-naphthylalanine
  SITE 4: note = D-arginine
  SITE 6: note = D-arginine
XXRXRXRQ

SEQ ID NO: 24 - protein, 8 aa, synthetic construct
  REGION 1..8: note = peptide is circular
  SITE 2: note = L-2-naphthylalanine
FXRRRRRQ

SEQ ID NO: 25 - protein, 9 aa, synthetic construct
  REGION 1..9: note = peptide is circular
  SITE 5: note = L-2-naphthylalanine
  SITE 8: note = L-norleucine
RRRRXFDXC

SEQ ID NO: 26 - protein, 5 aa, synthetic construct
  REGION 1..5: note = peptide is circular
  SITE 2: note = L-2-naphthylalanine
FXRRR

SEQ ID NO: 27 - protein, 5 aa, synthetic construct
  REGION 1..5: note = peptide is circular
FWRRR

SEQ ID NO: 28 - protein, 5 aa, synthetic construct
  REGION 1..5: note = peptide is circular
  SITE 4: note = L-2-naphthylalanine
RRRXF

SEQ ID NO: 29 - protein, 5 aa, synthetic construct
  REGION 1..5: note = peptide is circular
RRRWF

SEQ ID NO: 30 - protein, 7 aa, synthetic construct
  REGION 1..7: note = peptide is circular
  SITE 2: note = L-2-naphthylalanine
FXRRRRQ

SEQ ID NO: 31 - protein, 6 aa, synthetic construct
  REGION 1..6: note = peptide is circular
FFRRRQ

SEQ ID NO: 32 - protein, 6 aa, synthetic construct
  REGION 1..6: note = peptide is circular
  SITE 3: note = D-arginine
  SITE 5: note = D-arginine
FFXRXQ

SEQ ID NO: 33 - protein, 6 aa, synthetic construct
  REGION 1..6: note = peptide is circular
  SITE 4: note = D-arginine
FFRXRQ

SEQ ID NO: 34 - protein, 6 aa, synthetic construct
  REGION 1..6: note = peptide is circular
FRFRRQ

SEQ ID NO: 35 - protein, 6 aa, synthetic construct
  REGION 1..6: note = peptide is circular
FRRFRQ

SEQ ID NO: 36 - protein, 6 aa, synthetic construct
  REGION 1..6: note = peptide is circular
FRRRFQ

SEQ ID NO: 37 - protein, 6 aa, synthetic construct
  REGION 1..6: note = peptide is circular
  SITE 2: note = L-2-naphthylalanine
GXRRRQ

SEQ ID NO: 38 - protein, 6 aa, synthetic construct
  REGION 1..6: note = peptide is circular
FFFRAQ

SEQ ID NO: 39 - protein, 6 aa, synthetic construct
  REGION 1..6: note = peptide is circular
FFFRRQ

SEQ ID NO: 40 - protein, 7 aa, synthetic construct
  REGION 1..7: note = peptide is circular
FFRRRRQ

SEQ ID NO: 41 - protein, 7 aa, synthetic construct
  REGION 1..7: note = peptide is circular
FRRFRRQ

SEQ ID NO: 42 - protein, 7 aa, synthetic construct
  REGION 1..7: note = peptide is circular
FRRRFRQ

SEQ ID NO: 43 - protein, 7 aa, synthetic construct
  REGION 1..7: note = peptide is circular
RFFRRRQ

SEQ ID NO: 44 - protein, 7 aa, synthetic construct
  REGION 1..7: note = peptide is circular
RFRRFRQ

SEQ ID NO: 45 - protein, 7 aa, synthetic construct
  REGION 1..7: note = peptide is circular
FRFRRRQ

SEQ ID NO: 46 - protein, 7 aa, synthetic construct
  REGION 1..7: note = peptide is circular
FFFRRRQ

SEQ ID NO: 47 - protein, 6 aa, synthetic construct
  REGION 1..6: note = peptide is circular
FFRRRF

SEQ ID NO: 48 - protein, 7 aa, synthetic construct
  REGION 1..7: note = peptide is circular
FRFFRRQ

SEQ ID NO: 49 - protein, 7 aa, synthetic construct
  REGION 1..7: note = peptide is circular
RRFFFRQ

SEQ ID NO: 50 - protein, 7 aa, synthetic construct
  REGION 1..7: note = peptide is circular
FFRFRRQ

SEQ ID NO: 51 - protein, 7 aa, synthetic construct
  REGION 1..7: note = peptide is circular
FFRRFRQ

SEQ ID NO: 52 - protein, 7 aa, synthetic construct
  REGION 1..7: note = peptide is circular
FRRFFRQ

SEQ ID NO: 53 - protein, 7 aa, synthetic construct
  REGION 1..7: note = peptide is circular
FRRFRFQ

SEQ ID NO: 54 - protein, 7 aa, synthetic construct
  REGION 1..7: note = peptide is circular
FRFRFRQ

SEQ ID NO: 55 - protein, 7 aa, synthetic construct
  REGION 1..7: note = peptide is circular
RFFRFRQ

SEQ ID NO: 56 - protein, 7 aa, synthetic construct
  REGION 1..7: note = peptide is circular
  SITE 2: note = L-2-naphthylalanine
GXRRRRQ

SEQ ID NO: 57 - protein, 8 aa, synthetic construct
  REGION 1..8: note = peptide is circular
FFFRRRRQ

SEQ ID NO: 58 - protein, 8 aa, synthetic construct
  REGION 1..8: note = peptide is circular
RFFRRRRQ

SEQ ID NO: 59 - protein, 7 aa, synthetic construct
  REGION 1..7: note = peptide is circular
RRFFRRR

SEQ ID NO: 60 - protein, 8 aa, synthetic construct
  REGION 1..8: note = peptide is circular
RFFFRRRQ

SEQ ID NO: 61 - protein, 8 aa, synthetic construct
  REGION 1..8: note = peptide is circular
RRFFFRRQ

SEQ ID NO: 62 - protein, 8 aa, synthetic construct
  REGION 1..8: note = peptide is circular
FFRRFRRQ

SEQ ID NO: 63 - protein, 8 aa, synthetic construct
  REGION 1..8: note = peptide is circular
FFRRRRFQ

SEQ ID NO: 64 - protein, 8 aa, synthetic construct
  REGION 1..8: note = peptide is circular
FRRFFRRQ

SEQ ID NO: 65 - protein, 9 aa, synthetic construct
  REGION 1..9: note = peptide is circular
FFFRRRRRQ

SEQ ID NO: 66 - protein, 10 aa, synthetic construct
  REGION 1..10: note = peptide is circular
FFFRRRRRRQ

SEQ ID NO: 67 - protein, 7 aa, synthetic construct
  REGION 1..7: note = peptide is circular
  SITE 2: note = L-2-naphthylalanine
  SITE 4: note = D-arginine
  SITE 6: note = D-arginine
FXRXRXQ

SEQ ID NO: 68 - protein, 7 aa, synthetic construct
  REGION 1..7: note = peptide is circular
  VARIANT 1..2: note = Can be any naturally occurring amino acid
XXRRRRQ

SEQ ID NO: 69 - protein, 7 aa, synthetic construct
  REGION 1..7: note = peptide is circular
  SITE 2: note = D-phenylalanine
  SITE 5: note = D-arginine
FXFRXRQ

SEQ ID NO: 70 - protein, 7 aa, synthetic construct
  REGION 1..7: note = peptide is circular
  SITE 1: note = D-phenylalanine
  SITE 3: note = D-phenylalanine
  SITE 4: note = D-arginine
  SITE 6: note = D-arginine
FFFRRRQ

SEQ ID NO: 71 - protein, 7 aa, synthetic construct
  REGION 1..7: note = peptide is circular
  SITE 1: note = D-phenylalanine
  SITE 3: note = D-phenylalanine
  SITE 5: note = D-arginine
XFXRXRQ

SEQ ID NO: 72 - protein, 7 aa, synthetic construct
  REGION 1..7: note = peptide is circular
  SITE 2: note = D-phenylalanine
  SITE 4: note = D-arginine
  SITE 6: note = D-arginine
FXFXRXQ

SEQ ID NO: 73 - protein, 7 aa, synthetic construct
  REGION 1..7: note = peptide is circular
  SITE 1: note = D-phenylalanine
  SITE 3: note = L-2-naphthylalanine
  SITE 4: note = D-arginine
  SITE 6: note = D-arginine
FFXRRRQ

SEQ ID NO: 74 - protein, 7 aa, synthetic construct
  REGION 1..7: note = peptide is circular
  SITE 1: note = D-phenylalanine
  SITE 2: note = L-2-naphthylalanine
  SITE 3: note = D-phenylalanine
  SITE 4: note = D-arginine
  SITE 6: note = D-arginine
FXFRRRQ

SEQ ID NO: 75 - protein, 7 aa, synthetic construct
  REGION 1..7: note = peptide is circular
  SITE 1: note = L-2-naphthylalanine
  SITE 3: note = D-phenylalanine
  SITE 4: note = D-arginine
  SITE 6: note = D-arginine
XFFRRRQ

SEQ ID NO: 76 - protein, 6 aa, synthetic construct
  REGION 1..6: note = peptide is circular
  SITE 2: note = L-2-naphthylalanine
  SITE 3: note = D-arginine
  SITE 5: note = D-arginine
FXRRRQ

SEQ ID NO: 77 - protein, 6 aa, synthetic construct
  REGION 1..6: note = peptide is circular
  SITE 1: note = D-phenylalanine
  SITE 2: note = L-2-naphthylalanine
  SITE 3: note = D-arginine
  SITE 5: note = D-arginine
FXRRRQ

SEQ ID NO: 78 - protein, 8 aa, synthetic construct
  MOD_RES 1: note = Acetylation
  SITE 2: note = D-phenylalanine
  SITE 5: note = D-arginine
  SITE 7: note = D-arginine
KFFRRRRD

SEQ ID NO: 79 - protein, 8 aa, synthetic construct
  MOD_RES 1: note = Acetylation
  SITE 2: note = D-phenylalanine
  SITE 5: note = D-arginine
  SITE 7: note = D-arginine
  MOD_RES 1: note = L-2,3-diaminopropionic acid
XFFRRRRD

SEQ ID NO: 80 - protein, 8 aa, synthetic construct
  DISULFID 1..8: note = Intrachain via Cys-Cys
CWWRRRRC

SEQ ID NO: 81 - protein, 9 aa, synthetic construct
  DISULFID 1..9: note = Intrachain via Cys-Cys
CWWVRRRRC

SEQ ID NO: 82 - protein, 8 aa, synthetic construct
  DISULFID 1..8: note = Intrachain via Cys-Cys
CFWRRRRC

SEQ ID NO: 83 - protein, 8 aa, synthetic construct
  DISULFID 1..8: note = Intrachain via Cys-Cys
CWWWRRRC

SEQ ID NO: 84 - protein, 7 aa, synthetic construct
  REGION 1..7: note = peptide is circular
  SITE 1: note = L-homeoproline
  SITE 2: note = L-napthylalanine
  SITE 5..6: note = D-arginine
  SITE 7: note = D-glutamate
XXRERRE

SEQ ID NO: 85 - protein, 7 aa, synthetic construct
  REGION 1..7: note = peptide is circular
  SITE 1: note = L-homeoproline
  SITE 2: note = L-napthylalanine
  SITE 5..6: note = D-arginine
  SITE 7: note = D-glutamate
XXRRRRE

SEQ ID NO: 86 - protein, 7 aa, synthetic construct
  REGION 1..7: note = peptide is circular
  SITE 1: note = L-homeoproline
  SITE 2..3: note = L-napthylalanine
  SITE 5..6: note = D-arginine
  SITE 7: note = D-glutamate
XXXRRRE

SEQ ID NO: 87 - protein, 7 aa, synthetic construct
  REGION 1..7: note = peptide is circular
  SITE 1: note = L-homeoproline
  SITE 2..3: note = L-napthylalanine
  SITE 5..6: note = D-arginine
XXXRRRE

SEQ ID NO: 88 - protein, 7 aa, synthetic construct
  REGION 1..7: note = peptide is circular
  SITE 1: note = L-homeoproline
  SITE 2: note = L-napthylalanine
  SITE 5..6: note = D-arginine
  SITE 7: note = D-glutamate
XXFRRRE

SEQ ID NO: 89 - protein, 7 aa, synthetic construct
  REGION 1..7: note = peptide is circular
  SITE 1: note = L-homeoproline
  SITE 2: note = L-napthylalanine
  SITE 5..6: note = D-arginine
XXFRRRE

SEQ ID NO: 90 - protein, 7 aa, synthetic construct
  REGION 1..7: note = peptide is circular
  SITE 1: note = L-homeoproline
  SITE 2: note = L-napthylalanine
  SITE 3: note = D-phenylalanine
  SITE 5..6: note = D-arginine
  SITE 7: note = D-glutamate
XXFRRRE

SEQ ID NO: 91 - protein, 7 aa, synthetic construct
  REGION 1..7: note = peptide is circular
  SITE 1: note = L-homeoproline
  SITE 2: note = L-napthylalanine
  SITE 3: note = D-phenylalanine
  SITE 5..6: note = D-arginine
XXFRRRE

SEQ ID NO: 92 - protein, 7 aa, synthetic construct
  REGION 1..7: note = peptide is circular
  SITE 1: note = L-homeoproline
  SITE 2: note = L-napthylalanine
  SITE 3: note = D-napthylalanine
  SITE 5..6: note = D-arginine
XXXRRRE

SEQ ID NO: 93 - protein, 7 aa, synthetic construct
  REGION 1..7: note = peptide is circular
  SITE 1: note = L-homeoproline
  SITE 2: note = L-napthylalanine
  SITE 3: note = D-napthylalanine
  SITE 5..6: note = D-arginine
  SITE 7: note = D-glutamate
XXXRRRE

SEQ ID NO: 94 - protein, 8 aa, synthetic construct
  REGION 1..4: note = Cyclization between arginine is modified with pimelic acid and arginine is modified with lysine peptoid residue
  SITE 1..4: note = arginine is modified with pimelic acid and arginine is modified with lysine peptoid residue to create a cyclization
  SITE 4: note = arginine is modified with lysine peptoid residue
  SITE 1: note = arginine is modified with pimelic acid
RQRRGRRR

SEQ ID NO: 95 - protein, 19 aa, synthetic construct
  DISULFID 2..19: note = Intrachain via Cys-Cys
KCFQWQRNMRKVRGPPVSC

SEQ ID NO: 96 - protein, 11 aa, synthetic construct
  REGION 1..11: note = Cyclization between Lys and Glu
  SITE 2: note = D-arginine
  SITE 4: note = D-arginine
  SITE 6: note = D-arginine
  SITE 8: note = D-lysine
  SITE 10: note = D-arginine
KXRXGXKXRXE

SEQ ID NO: 97 - protein, 12 aa, synthetic construct
  REGION 1..12: note = Cyclization between Lys and Glu
  SITE 2: note = D-arginine
  SITE 4: note = D-arginine
  SITE 6: note = D-arginine
  SITE 8: note = D-arginine
  SITE 10: note = D-arginine
KXRXRXRXRXRE

SEQ ID NO: 98 - protein, 14 aa, synthetic construct
  SITE 13: note = D-proline
RVRTRGKRRIRRXP

SEQ ID NO: 99 - protein, 14 aa, synthetic construct
  SITE 13: note = D-proline
RTRTRGKRRIRVXP

SEQ ID NO: 100 - protein, 8 aa, synthetic construct
WRWRWRWR

SEQ ID NO: 101 - protein, 34 aa, synthetic construct
  DISULFID 4..21: note = Intrachain via Cys-Cys
  DISULFID 11..23: note = Intrachain via Cys-Cys
  DISULFID 17..29: note = Intrachain via Cys-Cys
GGVCPKILKKCRRDSDCPGACICRGNGYCGSGSD

SEQ ID NO: 102 - protein, 10 aa, synthetic construct
  REGION 1..10: note = Macrocyclization by multicomponent reaction with aziridine aldehyde and isocyanide
  SITE 2: note = L-3-cyclohexyl-alanine
  SITE 3: note = D-arginine
  SITE 4: note = L-3-cyclohexyl-alanine
  SITE 5: note = D-arginine
  SITE 6: note = L-3-cyclohexyl-alanine
  SITE 7: note = D-arginine
  SITE 8: note = L-3-cyclohexyl-alanine
  SITE 9: note = D-arginine
PXRXRXXXRG

SEQ ID NO: 103 - protein, 13 aa, synthetic construct
  SITE 1: note = Serine is modified with trimesic acid
  SITE 13: note = N-terminal amine and side chains of two L-2,3-diaminopropionic
  SITE 1..13: note = These positions are bicyclized with trimesic acid
  REGION 1..13: note = These positions are bicyclized with trimesic acid
  SITE 2: note = D-valine
  SITE 4: note = L-difluorophosphonomethyl phenylalanine
  SITE 6: note = MOD_BASE - L-2,3-diaminopropionic acid
  SITE 8: note = L-2-naphthylalanine
  SITE 13: note = L-2,3-diaminopropionic acid
SXPXHXFXRRRRX

SEQ ID NO: 104 - protein, 16 aa, synthetic construct
  SITE 1: note = D-alanine is modified with trimesic acid
  SITE 1: note = D-alanine
  SITE 16: note = N-terminal amine and side chains of two L-2,3-diaminopropionic
  REGION 1..16: note = Positions are bicyclized with trimesic acid
  SITE 16: note = Position is modified with trimesic acid
  SITE 2: note = MeGly
  SITE 3: note = D-phosphothreonine
  SITE 4: note = L-piperidine-2-carboxylic acid
  SITE 5: note = L-2-naphthylalanine
  SITE 8: note = D-alanine
  SITE 9: note = L-2,3-diaminopropionic acid
  SITE 11: note = L-2-naphthylalanine
  SITE 16: note = L-2,3-diaminopropionic acid
XXXXXRAXXFXRRRRX

SEQ ID NO: 105 - protein, 16 aa, synthetic construct
  SITE 1: note = Bound to C at position 8 with tris(bromomethyl)benzene
  SITE 8: note = Bound to C at positions 1 and 15 with tris(bromomethyl)benzene
  SITE 15: note = Bound to C at position 15 with tris(bromomethyl)benzene
  REGION 1..8: note = Cyclized with tris(bromomethyl)benzene
  REGION 8..15: note = Cyclized with tris(bromomethyl)benzene
CRRSRRGCGRRSRRCG

SEQ ID NO: 106 - protein, 5 aa, synthetic construct
  SITE 1..2: note = linked by dodecanoyl moiety
KRRRR

SEQ ID NO: 107 - protein, 16 aa, synthetic construct
LKKLCKLLKKLCKLAG

SEQ ID NO: 108 - protein, 9 aa, synthetic construct
  REGION 5..9: note = Cyclization between Lys and Glu
RRRRKRRRE

SEQ ID NO: 109 - protein, 9 aa, synthetic construct
  REGION 4..9: note = Cyclization between Lys and Glu
RRRKRRRRE

SEQ ID NO: 110 - protein, 9 aa, synthetic construct
  REGION 3..9: note = Cyclization between Lys and Glu
RRKRRRRRE

SEQ ID NO: 111 - protein, 9 aa, synthetic construct
  REGION 2..9: note = Cyclization between Lys and Glu
RKRRRRRRE

SEQ ID NO: 112 - protein, 8 aa, synthetic construct
CRCRCRCR

SEQ ID NO: 113 - protein, 12 aa, synthetic construct
  SITE 1: note = L-propargylglycine
  SITE 1: note = L-propargylglycine bound to L-6-Azido-2-amino-hexanoic by the click reaction to form cyclization
  REGION 12: note = L-6-Azido-2-amino-hexanoic bound to L-propargylglycine by the click reaction to form cyclization
  REGION 1..12: note = Cyclization by the click reaction between L-propargylglycine and L-6-Azido-2-amino-hexanoic
  SITE 12: note = L-6-Azido-2-amino-hexanoic
XLRKRLRKFRNX

SEQ ID NO: 114 - protein, 9 aa, synthetic construct
  REGION 2..4: note = L-2,3-diaminopropionic acid
  SITE 5: note = D-phenylalanine
  REGION 7..8: note = L-2,3-diaminopropionic acid
TXXXFLXXT

SEQ ID NO: 115 - protein, 9 aa, synthetic construct
  SITE 2: note = L-2-amino-3-guanidinylpropionic acid
  SITE 3: note = L-2,3-diaminopropionic acid
  SITE 4: note = L-2-amino-3-guanidinylpropionic acid
  SITE 5: note = D-phenylalanine
  REGION 7..8: note = L-2-amino-3-guanidinylpropionic acid
TXXXFLXXT

SEQ ID NO: 116 - protein, 7 aa, synthetic construct
  SITE 2: note = L-2-naphthylalanine
  REGION 1..7: note = peptide is circular
FXRRRRQ

SEQ ID NO: 117 - protein, 8 aa, synthetic construct
  REGION 1..8: note = peptide is circular
  SITE 2: note = D-phenylalanine
  SITE 3: note = L-2-naphthylalanine
  SITE 5: note = D-arginine
  SITE 7: note = D-arginine
FXXRXRXQ

SEQ ID NO: 118 - protein, 7 aa, synthetic construct
  REGION 1..7: note = peptide is circular
  SITE 1: note = D-phenylalanine
  SITE 2: note = L-2-naphthylalanine
  SITE 4: note = D-arginine
  SITE 6: note = D-arginine
FXRRRRQ

SEQ ID NO: 119 - protein, 8 aa, synthetic construct
  REGION 1..8: note = peptide is circular
  SITE 1: note = D-phenylalanine
  SITE 2: note = L-2-naphthylalanine
  SITE 4: note = D-arginine
  SITE 6: note = D-arginine
XXRXRXRQ

SEQ ID NO: 120 - protein, 7 aa, synthetic construct
  REGION 1..7: note = peptide is circular
  SITE 2: note = D-2-naphthylalanine
  SITE 3: note = D-arginine
  SITE 5: note = D-arginine
  SITE 7: note = D-glutamine
FXRRRRE

SEQ ID NO: 121 - protein, 7 aa, synthetic construct
  REGION 1..7: note = peptide is circular
  SITE 2: note = D-2-naphthylalanine
  SITE 3: note = D-arginine
  SITE 5: note = D-arginine
FXXRXRQ

SEQ ID NO: 122 - protein, 8 aa, synthetic construct
  REGION 1..8: note = peptide is circular
  SITE 2: note = L-2-naphthylalanine
FXRRRRRQ

SEQ ID NO: 123 - protein, 7 aa, synthetic construct
  REGION 1..7: note = peptide is circular
  SITE 5: note = L-2-naphthylalanine
RRFRXRQ

SEQ ID NO: 124 - protein, 8 aa, synthetic construct
  REGION 1..8: note = peptide is circular
  SITE 3: note = L-2-naphthylalanine
FFXRRRRQ

SEQ ID NO: 125 - protein, 8 aa, synthetic construct
  REGION 1..8: note = peptide is circular
  SITE 6: note = L-2-naphthylalanine
RFRFRXRQ

SEQ ID NO: 126 - protein, 6 aa, synthetic construct
  REGION 1..6: note = peptide is circular
  SITE 2: note = L-2-naphthylalanine
FXRRRQ

SEQ ID NO: 127 - protein, 7 aa, synthetic construct
  REGION 1..7: note = peptide is circular
  SITE 6: note = L-2-naphthylalanine
FRRRRXQ

SEQ ID NO: 128 - protein, 7 aa, synthetic construct
  REGION 1..7: note = peptide is circular
  SITE 1: note = D-arginine
  SITE 5: note = L-2-naphthylalanine
XRFRXRQ

SEQ ID NO: 129 - protein, 7 aa, synthetic construct
  REGION 1..7: note = peptide is circular
  SITE 3: note = L-2-naphthylalanine
RRXFRRQ

SEQ ID NO: 130 - protein, 200 aa, synthetic construct
  VARIANT 5..200: note = up to 195 residues may be absent in groups of 5
RRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRR

SEQ ID NO: 131 - protein, 10 aa, synthetic construct
  VARIANT 2..9: note = up to 5 may be absent
CRRRRRRRRC

SEQ ID NO: 132 - protein, 11 aa, synthetic construct
  SITE 2: note = bAla
  VARIANT 3..10: note = up to 5 may be absent
CXRRRRRRRRC

SEQ ID NO: 133 - protein, 11 aa, synthetic construct
  VARIANT 2..9: note = up to 5 positions may be absent
  SITE 10: note = bALA
CRRRRRRRRXC

SEQ ID NO: 134 - protein, 12 aa, synthetic construct
  VARIANT 2: note = Position can be any naturally occurring amino acid
  VARIANT 11: note = Position can be any naturally occurring amino acid
CXRRRRRRRRXC

SEQ ID NO: 135 - skipped

SEQ ID NO: 136 - protein, 7 aa, synthetic construct
CRRRRRC

SEQ ID NO: 137 - DNA, 21 bp, synthetic construct
  misc_feature 1..21: note = siLuc siRNA sequence
  misc_feature 1..21: note = DNA/RNA hybrid sequence
  misc_feature 2: note = t is uracil
  misc_feature 3: note = t is uracil
  misc_feature 8: note = t is uracil
  misc_feature 12: note = t is uracil
  misc_feature 15: note = t is uracil
  misc_feature 16: note = t is uracil
  misc_feature 1: note = Backbone is deoxyribosome at this position
  misc_feature 2: note = Backbone is ribosome at this position
  misc_feature 3: note = Backbone is ribosome at this position
  misc_feature 4..9: note = Backbone is deoxyribosome at this position
  misc_feature 8: note = Backbone is ribosome at this position
  misc_feature 9..11: note = Backbone is deoxyribosome at this position
  misc_feature 12: note = Backbone is ribosome at this position
  misc_feature 13..14: note = Backbone is deoxyribosome at this position
  misc_feature 15: note = Backbone is ribosome at this position
  misc_feature 16: note = Backbone is ribosome at this position
  misc_feature 17..21: note = Backbone is deoxyribosome at this position
  source 1..21: mol_type = other DNA
cttacgctgagtacttcgatt

SEQ ID NO: 138 - DNA, 21 bp, synthetic construct
  misc_feature 1..21: note = siLuc siRNA sequence
  misc_feature 1..21: note = DNA/RNA hybrid sequence
  misc_feature 1: note = t is uracil
  misc_feature 7: note = t is uracil
  misc_feature 10: note = t is uracil
  misc_feature 16: note = t is uracil
  misc_feature 1: note = Backbone is ribosome at this position
  misc_feature 2..6: note = Backbone is deoxyribosome at this position
  misc_feature 7: note = Backbone is ribosome at this position
  misc_feature 8..9: note = Backbone is deoxyribosome at this position
  misc_feature 10: note = Backbone is ribosome at this position
  misc_feature 11..15: note = Backbone is deoxyribosome at this position
  misc_feature 16: note = Backbone is ribosome at this position
  misc_feature 17..21: note = Backbone is deoxyribosome at this position
  source 1..21: mol_type = other DNA
tcgaagtactcagcgtaagtt

SEQ ID NO: 139 - DNA, 21 bp, synthetic construct
  modified_base 3: mod_base = cm
  modified_base 5: mod_base = cm
  modified_base 6: mod_base = um
  modified_base 10: mod_base = cm
  modified_base 12..13: mod_base = um
  modified_base 16: mod_base = um
  modified_base 17: mod_base = cm
  source 1..21: mol_type = other DNA
aangnngggngnnaannaatt

SEQ ID NO: 140 - DNA, 21 bp, synthetic construct
  misc_feature 1..21: note = DNA/RNA hybrid sequence
  misc_feature 1..21: note = DNA/RNA hybrid sequence
  misc_feature 1: note = t is uracil
  misc_feature 2: note = t is uracil
  misc_feature 5: note = t is uracil
  modified_base 6: mod_base = um
  modified_base 13: mod_base = cm
  misc_feature 18: note = t is uracil
  misc_feature 19: note = t is uracil
  misc_feature 1: note = Backbone is ribosome at this position
  misc_feature 2: note = Backbone is ribosome at this position
  misc_feature 3..4: note = Backbone is deoxyribosome at this position
  misc_feature 5: note = Backbone is ribosome at this position
  misc_feature 6
  misc_feature 7..17: note = Backbone is deoxyribosome at this position
  misc_feature 18: note = Backbone is ribosome at this position
  misc_feature 19: note = Backbone is ribosome at this position
  misc_feature 20..21: note = Backbone is deoxyribosome at this position
  source 1..21: mol_type = other DNA
ttgattaacgcccagcgtttt

SEQ ID NO: 141 - protein, 8 aa, synthetic construct
  REGION 1..8: note = synthetic peptide
  SITE 1: note = D-phenylalanine
  SITE 2: note = L-2-naphthylalanine
  SITE 4: note = D-arginine
  SITE 6: note = D-arginine
  SITE 7: note = MOD_BASE - A miniPEG (diethylene glycol) moiety is bound to the side chain of Q
  SITE 8: note = MOD_BASE - A miniPEG (diethylene glycol) moiety is bound to the CO2 of C
  REGION 7..8: note = Cyclization: Q and C are joined by a miniPEG (diethylene glycol) moiety
FXRRRRQC

SEQ ID NO: 142 - protein, 5 aa, synthetic construct
RRRRR

SEQ ID NO: 143 - protein, 6 aa, synthetic construct
RRRRRR

SEQ ID NO: 144 - protein, 20 aa, synthetic construct
RRRRRSSRRRRRRSSRRRRR

SEQ ID NO: 145 - protein, 6 aa, synthetic construct
RRRRRC

(19) United States
(12) Patent Application Publication
(10) Pub. No.: US 2026/0115301 A1
(43) Pub. Date: Apr. 30, 2026

(54) INTRACELLULAR DELIVERY OF NUCLEIC ACIDS

(71) Applicant: Ohio State Innovation Foundation, Columbus, OH (US)

(72) Inventors: Dehua PEI, Columbus, OH (US); Marina BUYANOVA, Columbus, OH (US); Ziqing QIAN, Columbus, OH (US)

(73) Assignee: Ohio State Innovation Foundation, Columbus, OH (US)

(21) Appl. No.: 19/329,431

(22) Filed: Sep. 15, 2025

Related U.S. Application Data
(63) Continuation of application No. 18/950,272, filed on Nov. 18, 2024.
Application No. 18/950,272 is a continuation of application No. 17/257,224, now Pat. No. 12,171,838.
(60) Provisional application No. 62/692,939, filed on Jul. 2, 2018.

(51) Int. Cl. A61K 47/64; C07K 7/64

(52) U.S. Cl. CPC A61K 47/6455; C07K 7/64

(57) ABSTRACT

The present disclosure provides polypeptide conjugates comprising a cell-penetrating peptide (CPP), directly or indirectly attached to a group comprising at least one peptide or polyamine, such as a polyarginine peptide.

### CROSS-REFERENCE TO RELATED APPLICATIONS

This application is a continuation of U.S. application Ser. No. 18/950,272, filed Nov. 18, 2024, which is a continuation of U.S. application Ser. No. 17/257,224, filed Dec. 30, 2020, which is a national stage application filed under 35 U.S.C. § 371 of PCT/US2019/040335 filed Jul. 2, 2019, which claims the benefit of priority to U.S. Provisional Application No. 62/692,939, filed Jul. 2, 2018. The entirety of each of these applications is hereby incorporated by reference for all purposes.

### STATEMENT REGARDING FEDERAL FUNDING

This invention was made with government support under GM110208 and GM122459 awarded by the National Institutes of Health. The government has certain rights in the invention.

### INCORPORATION BY REFERENCE

The Sequence Listing XML file named “25063-207US1_ST26” created on Sep. 15, 2025, and having a size of 282, 194 bytes, is hereby incorporated by reference.

### FIELD OF THE INVENTION

The present disclosure provides polypeptide conjugates comprising a cell-penetrating peptide (CPP), directly or indirectly attached to a group comprising at least one peptide or polyamine, such as a polyarginine peptide.

### BACKGROUND

Nucleic acids and their synthetic analogs hold enormous potential as therapeutic agents, especially against targets that are challenging for conventional drug modalities (e.g., intracellular protein-protein interactions and missing/defective proteins caused by genetic mutations). In the classical gene therapy approach, a gene of interest is incorporated into a viral vector or bacterial plasmid and delivered to tissues/cells to restore normal or correct for pathologic gene expression. More recently, a wide variety of strategies exploiting short oligonucleotides have been explored. For example, antisense oligonucleotides and small interfering RNAs (siRNAs) can be used to specifically knockdown virtually any single or group of genes. Splice-switching oligonucleotides, microRNAs, and anti-microRNAs can also enhance target gene expression or modulate/switch mRNA splicing to express the desired gene products. Another potentially very powerful approach is the use of gene-editing platforms (e.g., TALENs and CRISPR/CAS9) to alter the genomic DNA and provide a permanent cure of the disease. Finally, chemically stabilized mRNAs can be directly delivered into diseased cells and tissues and used as templates for protein synthesis. Collectively, these nucleic acid-based approaches greatly expand the space of pharmacologic targets which are otherwise undruggable with conventional drugs.

Despite their undoubted potential, clinical translation of nucleic acid-based drugs is limited by their poor bioavailability in the target tissues/cells. Because of their high molecular weights and negative charges (with the exception of a few oligonucleotide analogs), nucleic acids cannot cross the cellular membranes to reach the cell interior. Therefore, these nucleic acid-based molecules must be delivered into the target tissues/cells by an appropriate delivery system. The present disclosure is directed towards a novel nucleic acid delivery system which can effectively penetrate through the cellular membranes to reach the interior of the cells.

### SUMMARY OF THE INVENTION

In various embodiments, the present disclosure provides for polypeptide conjugates comprising:
a) a group that binds to a nucleic acid sequence by electrostatic interactions (P) comprising at least one peptide or polyamine; and
b) at least one cell-penetrating peptide (CPP);
wherein each peptide comprises at least three monomers selected from arginine, arginine-analog, lysine, lysine-analog, histidine, or histidine-analog; wherein the P is conjugated to the CPP through a bond or at least one linker (L); and
wherein the polypeptide conjugate is optionally charged.

In some embodiments, the polypeptide conjugate as disclosed herein has a molar ratio of P:CPP ranging from about 30:1 to about 1:2.

In some embodiments, the polypeptide conjugate as disclosed herein has an average molecular weight ranging from about 3 kDa to about 100 kDa.

In some embodiments, the polypeptide conjugate as disclosed herein has the following structure:

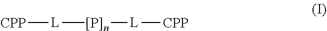

wherein n is an integer from 1 to 50; and
wherein P at each occurrence is same or different.

In some embodiments of the polypeptide conjugate as disclosed herein, P comprises a polyarginine peptide (pArg) comprising at least three monomers selected from arginine or arginine-analog.

In some embodiments of the polypeptide conjugate as disclosed herein, P comprises a polyamine selected from a spermidine polymer or a spermine polymer.

In some embodiments of the polypeptide conjugate as disclosed herein, at least one of the CPP is a cyclic CPP (cCPP). In some embodiments, each CPP is, independently, a cyclic CPP (cCPP). In some embodiments, the cCPP comprises from 4 to 14 amino acid monomers. In other embodiments, each cCPP is, independently, selected from Table 4.

In some embodiments of the polypeptide conjugate as disclosed herein, the cCPP is a cyclo(fΦRrRrQ) (SEQ ID NO: 118) peptide or a cyclo(FfΦRrRrQ) (SEQ ID NO: 16), wherein:
F is a L-phenylalanine;
f is a D-phenylalanine;
Φ is an L-2-naphthylalanine;
R is a L-arginine;
r is a D-arginine; and
Q is a L-glutamine.

In some embodiments of the polypeptide conjugate as disclosed herein, the pArg comprises at least five arginine monomers or arginine-analog monomers. In some embodiments, the pArg further comprises at least one cysteine monomer.

In some embodiments, the polypeptide conjugate as disclosed herein has the following structure:

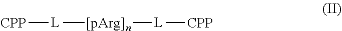

wherein the [pArg]ₙ (SEQ ID NO: 130) is

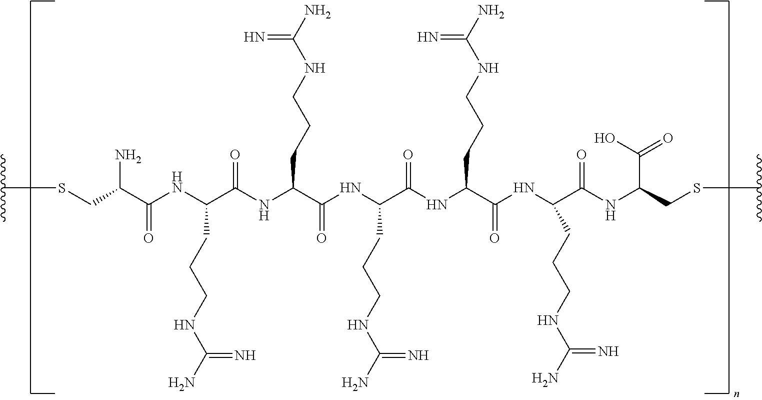

or a charged species thereof.

In some embodiments of the polypeptide conjugate as disclosed herein, n is an integer 1 to 40.

In some embodiments of the polypeptide conjugate as disclosed herein, at least one L comprises a divalent optionally substituted group selected from amino acid, alkylene, alkenylene, alkynylene, aryl, cycloalkyl, cycloalkenyl, cycloalkynyl, heterocyclyl, heteroaryl, (R¹—X—R²)z-, or combinations thereof; wherein
each of R¹ and R² are independently selected from a bond, alkylene, alkenylene, alkynylene, carbocyclyl, and heterocyclyl, wherein R¹ and R² are not both a bond;
each X is independently N, S, and O; and
z is an integer selected from 1 to 20.

In some embodiments of the polypeptide conjugate as disclosed herein, at least one L comprises an optionally substituted —(O—CH₂CH₂)z- or an optionally substituted —(CH₂CH₂—O)z-. In some embodiments, at least one L comprises a divalent 8-amino-3,6-dioxaoctanoic acid residue. In other embodiments, at least one L comprises a divalent 8-amino-3,6,9-trioxaundecanoic acid residue.

In some embodiments of the polypeptide conjugate as disclosed herein, at least one L comprises a physiological cleavable group (PCG). In some embodiments, each PCG is, independently, selected from —S—S—, carbonate, thiocarbonate, thioester, sulfoxide, hydrazine, or protease-cleavable dipeptide linker. In other embodiments, each PCG comprises at least one —S—S—.

In some embodiments of the polypeptide conjugate of formula (I), at least one of the “-” between L and [P]ₙ represents a bond between two sulfur atoms (disulfide bond). In other embodiments, the “-” between each L and [P]ₙ represents a bond between two sulfur atoms (disulfide bond).

In some embodiments of the polypeptide conjugate disclosed herein, each P, independently, further comprises at least one group selected from:

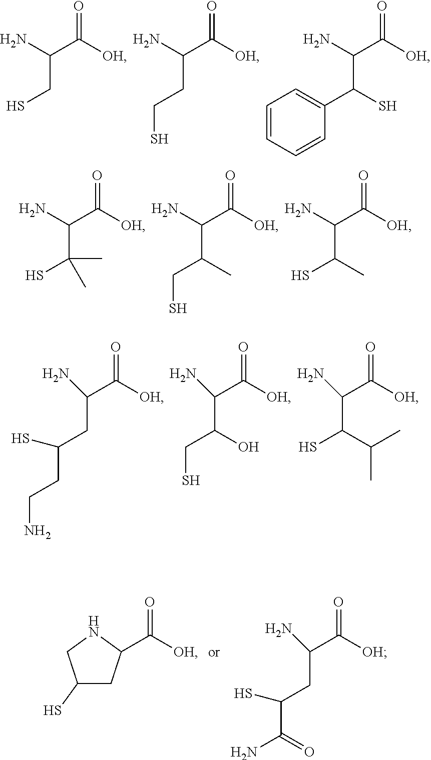

wherein the bond to the hydrogen on at least one of the N- or C-termini is replaced by a bond to the peptide or polyamine; and
wherein the bond to the hydrogen on the thiol group is replaced by a bond to the CPP (or cCPP)

In some embodiments of the polypeptide conjugate disclosed herein, each P, independently, further comprises at least one group selected from:

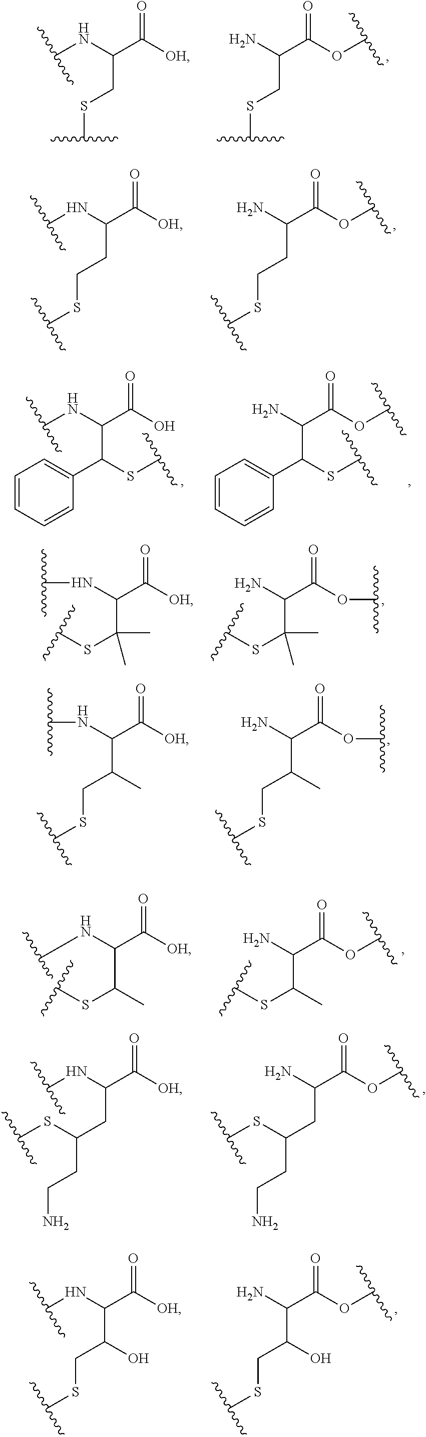

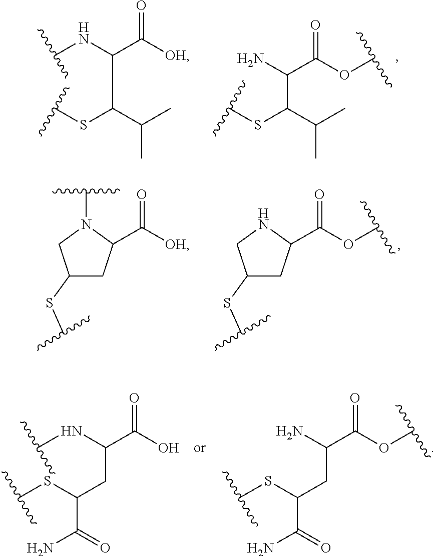

In some embodiments of the polypeptide conjugate disclosed herein, at least one of the P further comprises

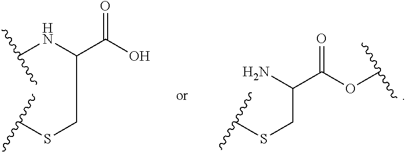 [SMILES: *NC(CS*)C(=O)O ; *OC(=O)C(N)CS*]

In other embodiments, at least one of the P further comprises at least two groups selected form

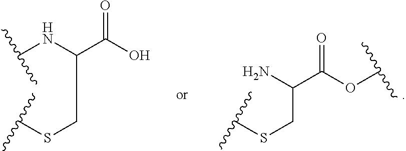 [SMILES: *NC(CS*)C(=O)O ; *OC(=O)C(N)CS*]

In some embodiments, the polypeptide conjugate as disclosed herein has the following structure:

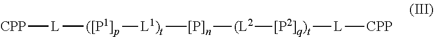

wherein
n, p, and q are each independently an integer from 1 to 50;
tis each independently 0 or 1;
P¹ and P² each comprises at least one peptide or polyamine, wherein each peptide comprises at least three monomers selected from arginine, arginine-analog, lysine, lysine-analog, histidine, or histidine-analog, wherein P, P¹ and P², at each occurrence, are same or different; and
L¹ and L² are each independently absent or L as defined in claim 1, wherein L, L¹, and L², at each occurrence, are same or different.

In some embodiments of the polypeptide conjugate of formula (III), P, P¹ or P² comprises a polyarginine peptide (pArg) comprising at least three monomers selected from arginine or arginine-analog. In other embodiments, P, P¹ or P² comprises a polyamine selected from a spermidine polymer or a spermine polymer.

In some embodiments of the polypeptide conjugate of formula (III), at least one of the CPP is a cyclic CPP (cCPP). In other embodiments, each CPP is, independently, a cyclic CPP (cCPP). In one embodiment, the cCPP comprises from 4 to 14 amino acid monomers. In another embodiment, each cCPP is, independently, selected from Table 4.

In some embodiments of the polypeptide conjugate of formula (III) as disclosed herein, the cCPP is a cyclo(fΦRrRrQ) (SEQ ID NO: 118) peptide or a cyclo(FfΦRrRrQ) (SEQ ID NO: 16), wherein:
F is a L-phenylalanine;
f is a D-phenylalanine;
Φ is an L-2-naphthylalanine;
R is a L-arginine;
r is a D-arginine; and
Q is a L-glutamine.

In some embodiments of the polypeptide conjugate of formula (III), the pArg comprises at least five arginine monomers or arginine-analog monomers. In one embodiment, the pArg further comprises at least one cysteine monomer.

In some embodiments of the polypeptide conjugate of formula (III), n is an integer 1 to 40. In some embodiments of the polypeptide conjugate of formula (III), n is an integer 5 to 40.

In some embodiments of the polypeptide conjugate of formula (III), at least one of L, L¹, or L² comprises a divalent optionally substituted group selected from amino acid, polyethylene glycol, alkylene, alkenylene, alkynylene, aryl, cycloalkyl, cycloalkenyl, cycloalkynyl, heterocyclyl, heteroaryl, —(R¹—X—R²)z-, or combinations thereof;
each of R¹ and R² are independently selected from a bond, alkylene, alkenylene, alkynylene, carbocyclyl, and heterocyclyl, wherein R¹ and R² are not both a bond;
each X is independently N, S, and O; and
z is an integer selected from 1 to 20.

In some embodiments of the polypeptide conjugate of formula (III), at least one of L, L¹, or L² comprises an optionally substituted —(O—CH₂CH₂)z- or an optionally substituted —(CH₂CH₂—O)z-.

In some embodiments of the polypeptide conjugate of formula (III), at least one of L, L¹, or L² comprises a divalent 8-amino-3,6-dioxaoctanoic acid residue. In some embodiments, at least one of L, L¹, or L² comprises a divalent 8-amino-3,6,9-trioxaundecanoic acid residue. In other embodiments, at least one of L, L¹, or L² comprises a physiological cleavable group (PCG).

In some embodiments of the polypeptide conjugate of formula (III) as disclosed herein, each PCG is, independently, selected from —S—S—, carbonate, thiocarbonate, thioester, sulfoxide, hydrazine, or protease-cleavable dipeptide linker. In some embodiments, each PCG comprises at least one —S—S—.

In some embodiments of the polypeptide conjugate of formula (III), at least one of the “-” between L and ([P¹]ₚ-L¹)ₜ, L and [P]ₙ, or L and (L²-[P²]_{q})ₜ represents a bond between two sulfur atoms (disulfide bond).

In some embodiments of the polypeptide conjugate of formula (III), at least one of P, P¹ or P² further comprises at least one group selected from:

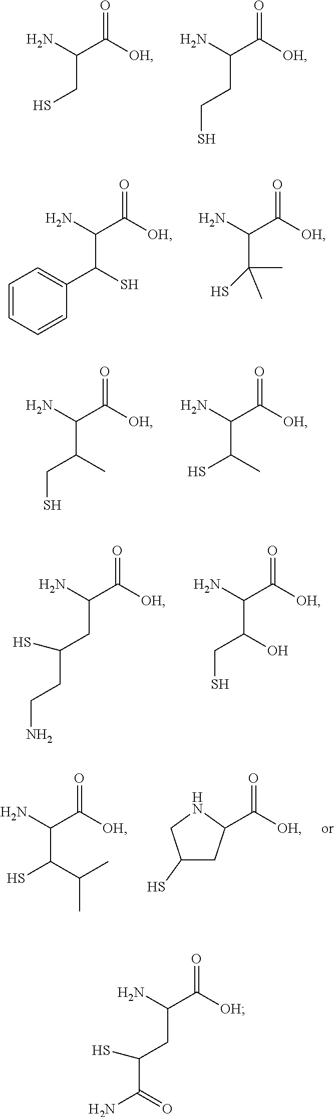

wherein the bond to the hydrogen on at least one of the N- or C-termini is replaced by a bond to the peptide or polyamine; and
wherein the bond to the hydrogen on the thiol group is replaced by a bond to the CPP.

In some embodiments of the polypeptide conjugate of formula (III), at least one of P, P¹ or P² further comprises at least one group selected from:

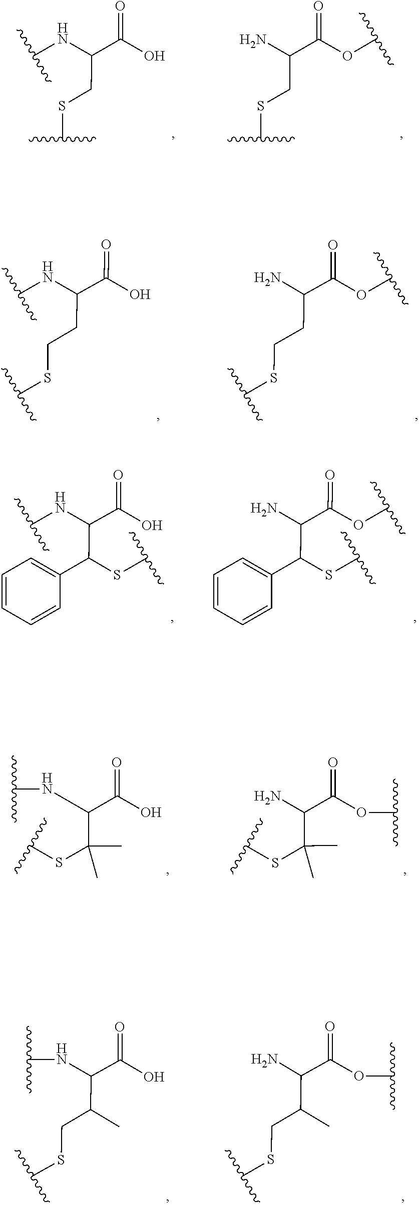

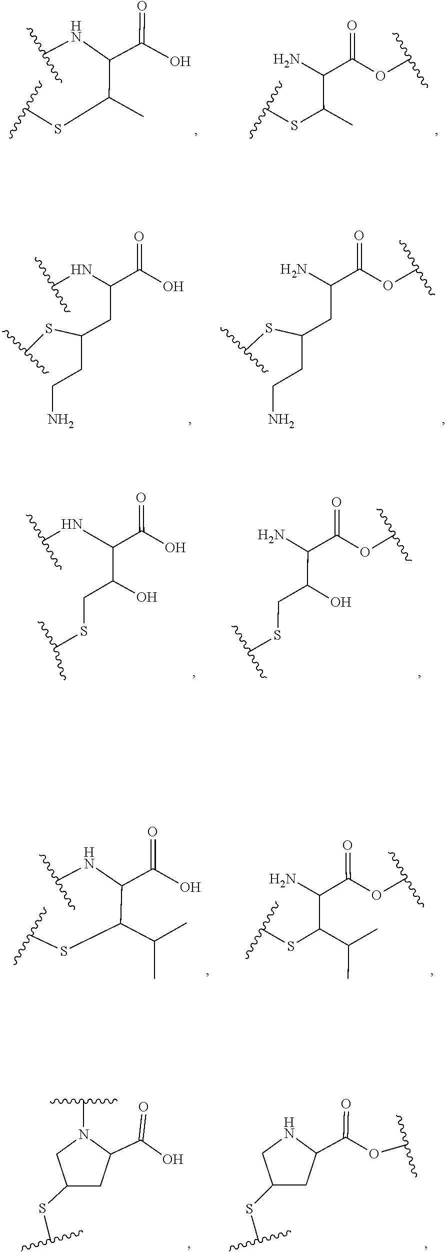

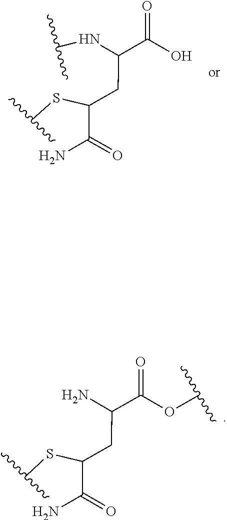 [SMILES: *NC(CC(S*)C(N)=O)C(=O)O ; *OC(=O)C(N)CC(S*)C(N)=O]

In some embodiments of the polypeptide conjugate of formula (III), at least one of P, P¹ or P² further comprises

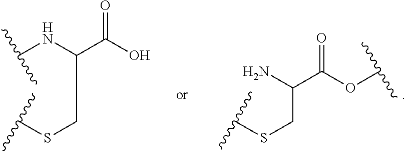 [SMILES: *NC(CS*)C(=O)O ; *OC(=O)C(N)CS*]

In other embodiments, at least two of P, P¹ or P² further comprises a group selected form

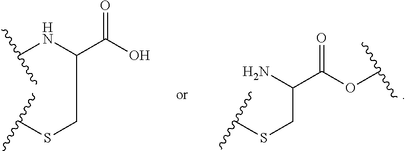 [SMILES: *NC(CS*)C(=O)O ; *OC(=O)C(N)CS*]

In some embodiments of the polypeptide conjugate of formula (III), the pArg further comprises one or more β-alanine monomers. In some embodiments, each pArg comprises, independently, 3, 4, 5, 6, 7, or 8 arginine monomers. In other embodiments, each pArg comprises 5 arginine monomers. In one embodiment, at least one pArg further comprises a cysteine monomer.

In some embodiments of the polypeptide conjugate of formula (I), (II), or (III), the pArg is each independently selected from: -Cys-(Arg)_{X}-Cys- (SEQ ID NO: 131), -Cys-βAla-(Arg)_{X}-Cys- (SEQ ID NO: 132), -Cys-(Arg)_{X}-βAla-Cys- (SEQ ID NO: 133), or -Cys-βAla-(Arg)_{X}-βAla-Cys- (SEQ ID NO: 134), wherein x=3, 4, 5, 6, 7, or 8.

In some embodiments of the polypeptide conjugate of formula (I), (II), or (III), P is each independently selected from: -Cys-(Arg)_{X}-Cys- (SEQ ID NO: 131), -Cys-βAla-(Arg)_{X}-Cys- (SEQ ID NO: 132), -Cys-(Arg)_{X}-βAla-Cys- (SEQ ID NO: 133), or -Cys-βAla-(Arg)_{X}-βAla-Cys- (SEQ ID NO: 134), -Cys-(polyamine)_{X}-Cys-, -Cys-βAla-(polyamine)_{X}-Cys-, -Cys-(polyamine)_{X}-βAla-Cys-, -Cys-βAla-(polyamine)_{X}-βAla-Cys- (SEQ ID NO: 135), —S-(polyamine)_{X}-S—, wherein x=3, 4, 5, 6, 7, or 8.

In some embodiments, the polypeptide conjugate of formula (I) as disclosed herein has the structure

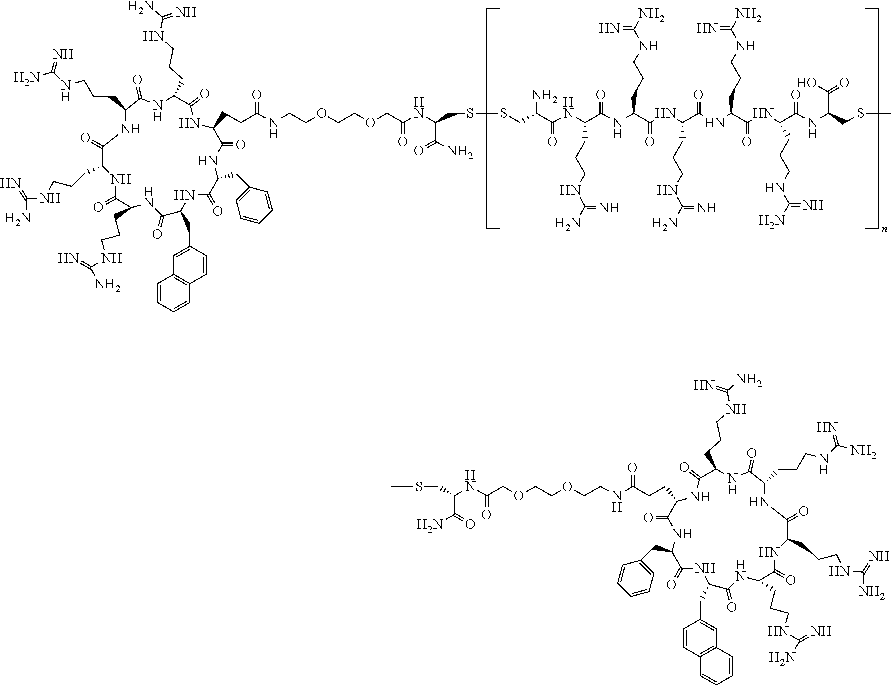

or a charged species thereof.

In some embodiments, the polypeptide conjugate of formula (I) as disclosed herein has the structure

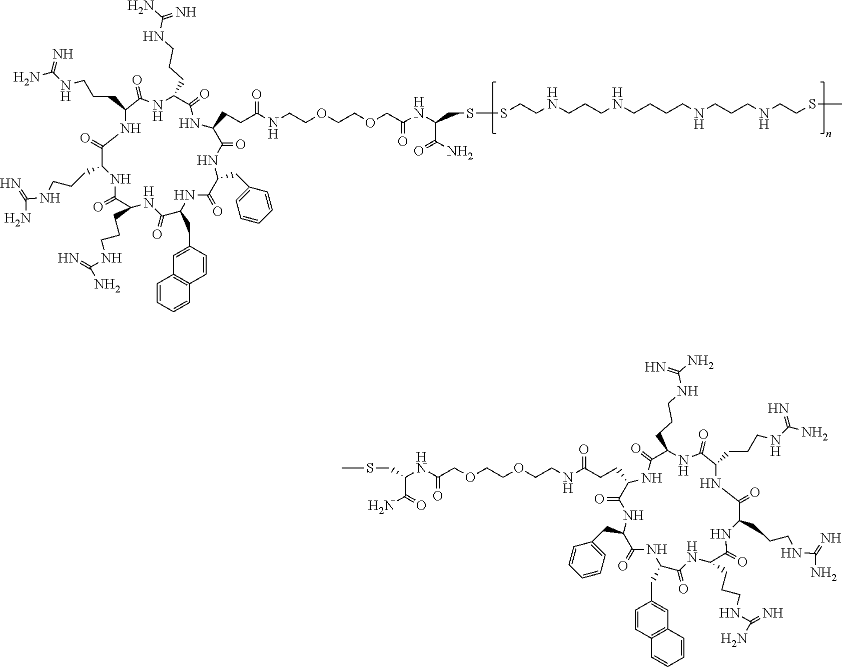

or a charged species thereof.

In some embodiments, the polypeptide conjugate as disclosed herein has the following structure:

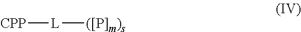

wherein:
s is an integer from 1 to 10;
each m is, independently, an integer from 1 to 50; and
wherein P at each occurrence is same or different.

In some embodiments of the polypeptide conjugate of formula (IV), P comprises a polyarginine peptide (pArg) comprising at least three monomers selected from arginine or arginine-analog. In some embodiments, P comprises a polyamine selected from a spermidine polymer or a spermine polymer.

In some embodiments of the polypeptide conjugate of formula (IV), the CPP is a cyclic CPP (cCPP). In some embodiments, the cCPP comprises from 4 to 14 amino acid monomers. In other embodiments, the cCPP is selected from Table 4.

In some embodiments of the polypeptide conjugate of formula (IV), the pArg comprises at least five arginine monomers or arginine-analog monomers. In some embodiments, the pArg further comprises at least one cysteine monomer.

In some embodiments of the polypeptide conjugate of formula (IV), L comprises a divalent optionally substituted group selected from amino acid, polyethylene glycol, alkylene, alkenylene, alkynylene, aryl, cycloalkyl, cycloalkenyl, cycloalkynyl, heterocyclyl, heteroaryl, —(R¹—X—R²)z-, or combinations thereof;
each of R¹ and R² are independently selected from a bond, alkylene, alkenylene, alkynylene, carbocyclyl, and heterocyclyl, wherein R¹ and R² are not both a bond;
each X is independently N, S, and O; and
z is an integer selected from 1 to 20.

In some embodiments of the polypeptide conjugate of formula (IV), L comprises an optionally substituted —(O—CH₂CH₂)z- or an optionally substituted —(CH₂CH₂—O)z-. In some embodiments, L comprises a divalent 8-amino-3,6-dioxaoctanoic acid residue. In other embodiments, L comprises a divalent 8-amino-3,6,9-trioxaundecanoic acid residue. In one embodiment, L comprises a physiological cleavable group (PCG).

In some embodiments of the polypeptide conjugate of formula (IV), each PCG is, independently, selected from —S—S—, carbonate, thiocarbonate, thioester, sulfoxide, hydrazine, or protease-cleavable dipeptide linker. In some embodiments, each PCG comprises at least one —S—S—.

In some embodiments of the polypeptide conjugate of formula (IV), L comprises a polythiolamine or a 3,5-bis(mercaptomethyl)benzoyl (Bmb) amide.

In some embodiments of the polypeptide conjugate of formula (IV), L comprises two or more physiological cleavable groups.

In some embodiments of the polypeptide conjugate of formula (IV), the “-” between L and ([P]ₘ)ₛ represents a bond between two sulfur atoms (disulfide bond).

In some embodiments of the polypeptide conjugate of formula (IV), each P, independently, further comprises at least one group selected from:

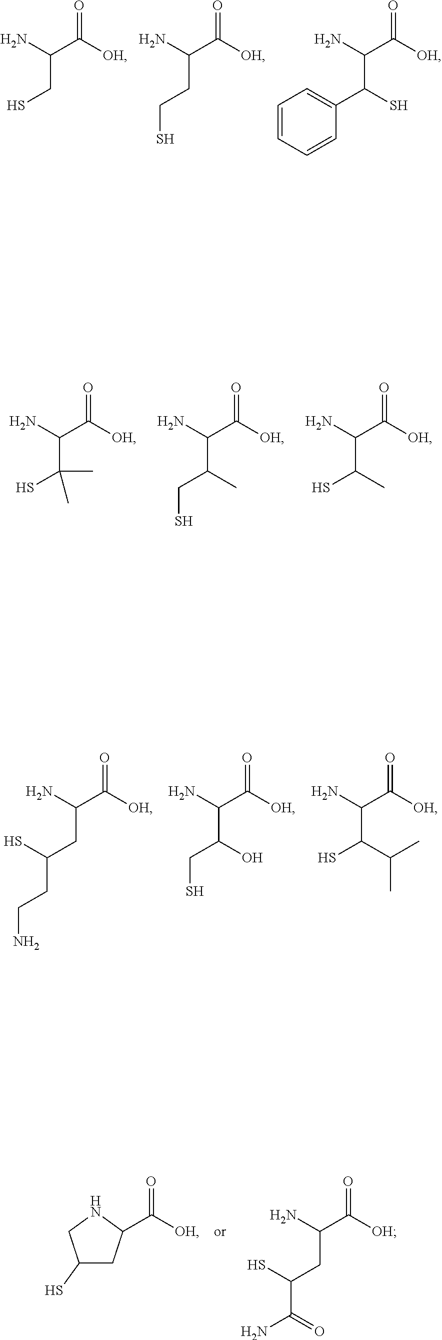

wherein the bond to the hydrogen on one at least one of the N- or C-termini is replaced by a bond to the peptide or polyamine; and
wherein the bond to the hydrogen on the thiol group is replaced by a bond to the CPP.

In some embodiments of the polypeptide conjugate of formula (IV), each P, independently, further comprises at least one group selected from:

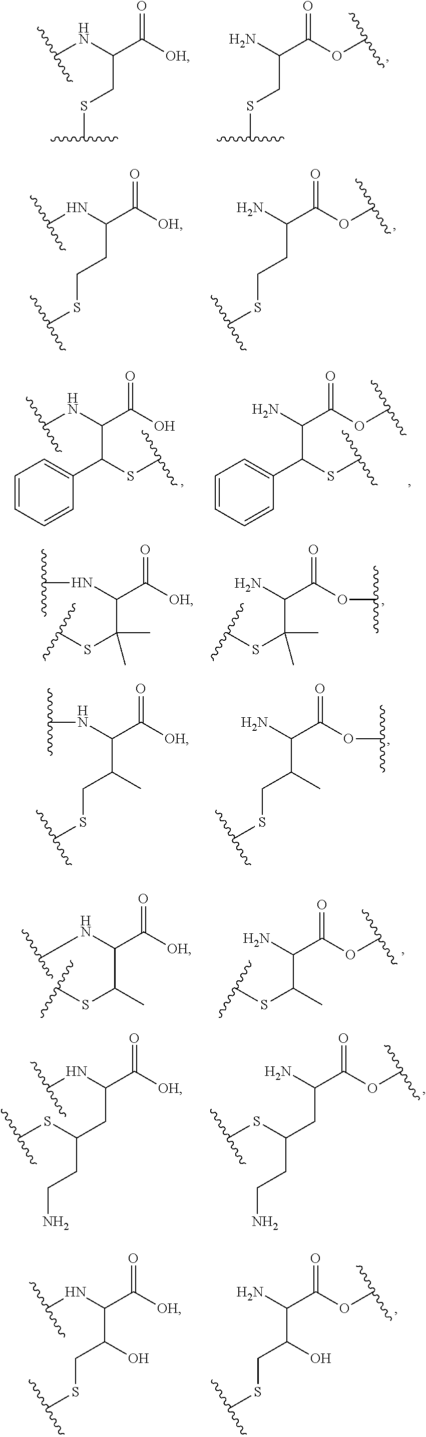

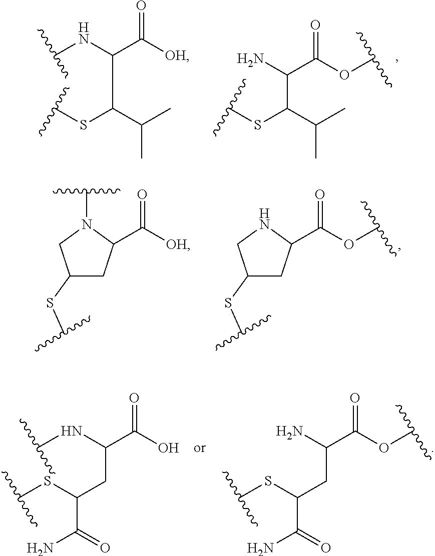

In some embodiments of the polypeptide conjugate of formula (IV), at least one of the P further comprises

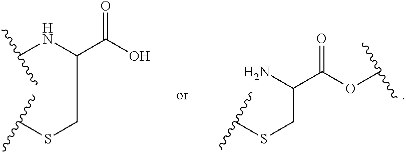 [SMILES: *NC(CS*)C(=O)O ; *OC(=O)C(N)CS*]

In other embodiments, at least one of the P further comprises at least two groups selected from

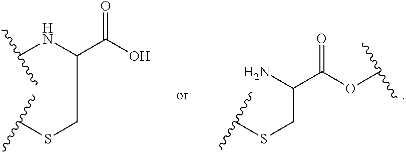 [SMILES: *NC(CS*)C(=O)O ; *OC(=O)C(N)CS*]

In some embodiments of the polypeptide conjugate of formula (IV), s is 2, 3, 4, or 5.

In some embodiments, the polypeptide conjugate of formula (IV) as disclosed herein has the structure

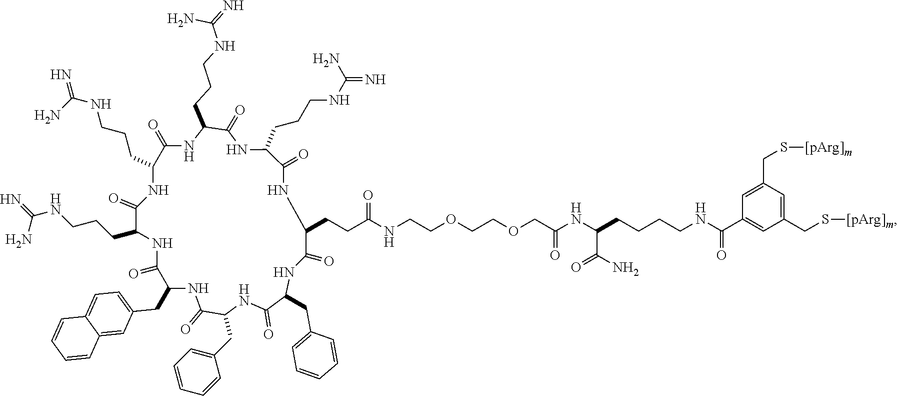

or a charged species thereof.

In some embodiments, the polypeptide conjugate of formula (IV) as disclosed herein has the structure

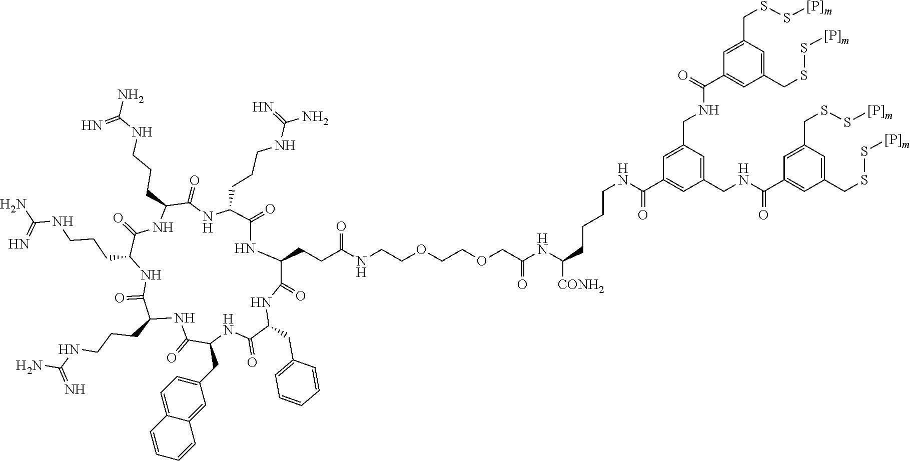

or a charged species thereof.

In some embodiments, the polypeptide conjugate as disclosed herein has the following structure:

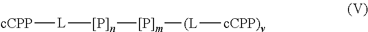

wherein,
cCPP is a cyclic CPP; and
n is an integer selected from 1 to 50;
m is an integer selected from 0 to 49 provided that the sum of n and m is 50 or less;
v is 0 or 1;
wherein P at each occurrence is same or different; and
wherein when v is 0, the last [P] in [P]ₘ is monovalent.

In some embodiments of the polypeptide conjugate of formula (V), the “-” between [P]ₙ and [P]ₘ represents a bond between two sulfur atoms (a disulfide bond).

In some embodiments, the polypeptide conjugate of formula (IV) has the following structure:

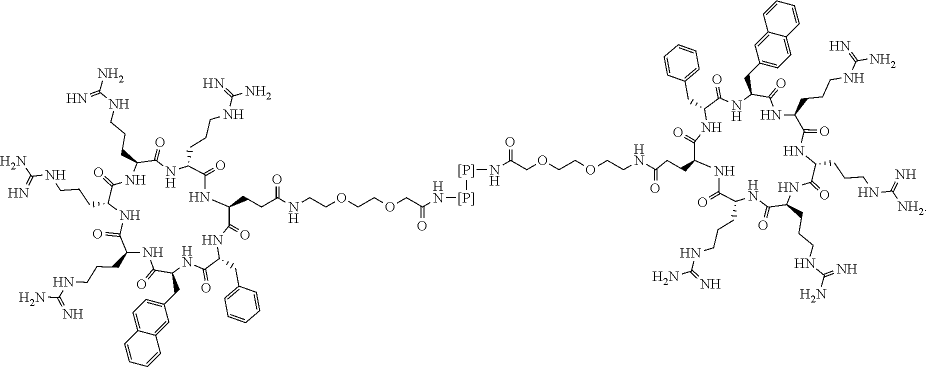

or a charged species thereof

### (VI).

In some embodiments of the polypeptide conjugate of formula (VI), the “-” between [P] and [P] represents a bond between two sulfur atoms (a disulfide bond).

In some embodiments, the polypeptide conjugate as disclosed herein has the following structure:

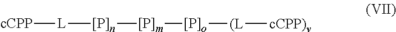

wherein,
cCPP is a cyclic CPP; and
n is an integer selected from 1 to 50;
m and o, are each independently, an integer from 0 to 49 provided that the sum of n, m, and o is 50 or less;
v is 0 or 1;
wherein P at each occurrence is same or different; and
wherein when v is 0, the last [P] in [P]ₒ is monovalent.

In some embodiments of the polypeptide conjugate of formula (VII) as disclosed herein, at least one of the “-” between [P]ₙ and [P]ₘ or [P]ₘ and [P]ₒ represents a bond between two sulfur atoms (a disulfide bond). In other embodiments, the “-” between [P]ₙ and [P]ₘ and between [P]ₘ and [P]ₒ represents a bond between two sulfur atoms (a disulfide bond).

In some embodiments, the polypeptide conjugate of formula (VI) as disclosed herein, has the following structure:

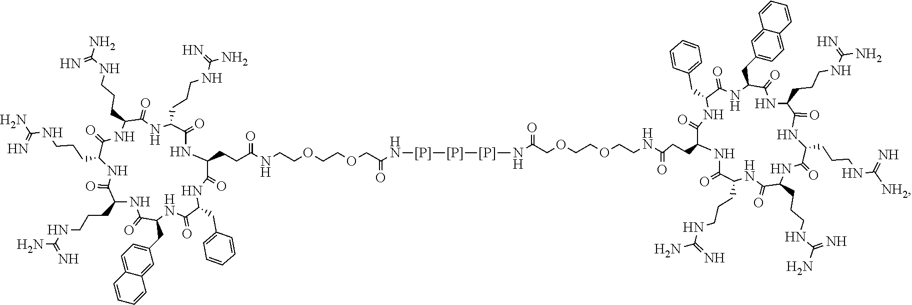

or a charged species thereof

### (VIII)

In some embodiments of the polypeptide conjugate of formula (VIII) as disclosed herein, at least one of the “-” between [P] and [P] represents a bond between two sulfur atoms (a disulfide bond). In other embodiment, each “-” between [P] and [P] represents a bond between two sulfur atoms (a disulfide bond).

The present disclosure also relates to a complex comprising any one of the polypeptide conjugates as disclosed herein and at least one nucleic acid sequence.

The present disclosure also relates to a cell comprising any one of the polypeptide conjugates as disclosed herein.

The present disclosure also relates to a cell comprising a complex comprising any one of the polypeptide conjugates as disclosed herein and a nucleic acid sequence.

The present disclosure also relates to a method of delivering a nucleic acid sequence to a cell, comprising contacting the cell with any one of the complexes as disclosed herein.

The present disclosure also relates to a method of delivering a nucleic acid sequence to a cell of a subject in need thereof, comprising administering any one of the complexes as disclosed herein.

The present disclosure also relates to a method of treating a disease or condition in a patient in need thereof, comprising administering any one of the complexed as disclosed herein to the patient.

### BRIEF DESCRIPTION OF FIGURES

FIG. 1 shows a design concept of polypeptide conjugate of the disclosure.

FIG. 2 shows an analytical HPLC (high-performance liquid chromatography) trace of polypeptide conjugate synthesized in Example 1. The arrows to CRC and cyclo(fΦRrRrQ) (SEQ ID NO: 118) points to HPLC trace of conjugate after reduction with 10 mM DTT (dithiothreitol).

FIG. 3 shows ¹H NMR (nuclear magnetic resonance) spectrum of the polypeptide conjugate synthesized in Example 1.

FIG. 4A shows gel-filtration chromatography traces of poly-L-lysine standard.

FIG. 4B shows gel-filtration chromatography traces of the polypeptide conjugate synthesized in Example 1 (CRC5—top line at t=9 min; CRC10 bottom line at t=9 min).

FIG. 5A shows the effect of CRC5 polymer synthesized in Example 1 on HeLa cells as assayed by the MTT (3-(4,5-dimethylthiazol-2-yl)-2,5-diphenyltetrazolium bromide) test.

FIG. 5B shows the effect of lipofectamine on Hela cells as assayed by the MTT

FIG. 6 shows live-cell confocal microscope images of HeLa cells treated with the CRC5/5′-FAM-siLuc complex (Example 1) at 3 □M siRNA in OptiMEM (top) or at 1 □M siRNA in DMEM with 1% FBS, 1% Abs (bottom). Left, GFP channel; Center, DIC; and right, overlap of the above.

FIG. 7 shows knockdown of luciferase expression in HeLa-Luc cells by CRC5/siLuc complexes (Example 1, 75 nM), the individual components, and lipofectamine/siLuc complex at 24 h and 48 h after treatment.

FIG. 8 shows MALDI-TOF MS spectra of CPP12-(R₅)₂ (top), CPP12-(R₁₀)₂ (middle), and CPP12-(R₁₅)₂ (bottom).

FIG. 9 shows live-cell confocal microscope images of Hela cells treated with the CPP9-(R₅)₂/5′-FAM-siLuc complex (Example 2) at 3 □M siRNA. Center, GFP channel; Right, DIC; and left, overlap of the above.

FIG. 10 shows knockdown of luciferase expression in HeLa-Luc cells by CPP12-(Rₙ)₂/siLuc complexes (Example 2, 75 nM) and lipofectamine/siLuc complex at 48 h after treatment.

FIG. 11 shows MALDI-TOF MS spectra of (CPP9-R₅)₂ (top) and (CPP9-R₈)₂ (bottom).

FIG. 12 shows live-cell confocal microscope images of HeLa cells treated with the (CPP9-R₅)₂/5′-FAM-siLuc complex (Example 3) at 3 μM siRNA. Center, GFP channel; Right, DIC; and left, overlap of the above.

FIG. 13 shows knockdown of luciferase expression in HeLa-Luc cells by CPP9-Rₙ)₂/siLuc complexes (Example 3, 75 nM) and lipofectamine/siLuc complex at 48 h after treatment.

FIG. 14 shows MALDI-TOF MS spectra of the polypeptide conjugate synthesized according to Example 4.

FIG. 15 shows the effect of the polypeptide conjugate synthesized according to Example 4 on HeLa cells as assayed by the MTT test.

FIG. 16 shows live-cell confocal microscope images of HeLa cells treated with the polypeptide conjugate synthesized according to Example 4/5′-FAM-siLuc complex at 1 μM siRNA in DMEM with 1% FBS (top) or 3 μM siRNA in OptiMEM (bottom). Center, GFP channel; Right, DIC; and left, overlap of the above.

FIG. 17 shows knockdown of luciferase expression in HeLa-Luc cells by the polypeptide conjugate of Example 4/siLuc complexes (Example 4, 75 nM) and lipofectamine/siLuc complex at 48 h after treatment.

FIG. 18 is a graph of gene expression (%) with cells treated with SiLuc and either Lipo2000 or CRC5.

FIG. 19 is a graph of gene expression (%) with cells treated with SiGFP and either Lipo2000 or CRC5.

### DETAILED DESCRIPTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### Definitions

While the following terms are believed to be well understood by one of ordinary skill in the art, the following definitions are set forth to facilitate explanation of the presently disclosed subject matter.

Throughout the present specification, the terms “about” and/or “approximately” may be used in conjunction with numerical values and/or ranges. The term “about” is understood to mean those values near to a recited value, as well as the recited value.

Throughout the present specification, numerical ranges are provided for certain quantities. It is to be understood that these ranges comprise all values and subranges therein. Thus, the range “from 50 to 80” includes all possible values therein (e.g., 50, 51, 52, 53, 54, 55, 56, etc.) and all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “a polypeptide conjugate” refers to one or more polypeptide conjugates or at least one polypeptide conjugate. As such, the terms “a” (or “an”), “one or more” and “at least one” are used interchangeably herein. In addition, reference to “a polypeptide conjugate” by the indefinite article “a” or “an” does not exclude the possibility that more than one of the polypeptide conjugates is present, unless the context clearly requires that there is one and only one of the polypeptide conjugates.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

As used herein, “treat,” “treating,” “treatment” and variants thereof, refers to any administration of the polypeptide conjugate of the present disclosure that partially or completely alleviates, ameliorates, prevents, relieves, inhibits, delays onset of, reduces severity of, and/or reduces incidence of one or more symptoms or features of a disease or a condition as described herein.

As used herein, “therapeutically effective” refers to an amount of the polypeptide conjugate or the complex thereof of the present disclosure that can deliver an amount of a therapeutic nucleic acid which confers a therapeutic effect on a patient.

As used herein, “cell penetrating peptide” or “CPP” refers to any peptide which is capable of penetrating a cell membrane. As used herein, “cyclic cell penetrating peptide” or “cCPP” refers to any cyclic peptide which is capable of penetrating a cell membrane.

As used herein, “linker” or “L” refers to a moiety that covalently attaches two or more components of the polypeptide conjugates disclosed herein (e.g., a linker may covalently attach a CPP and a group that binds to a nucleic acid sequence by electrostatic interactions [i.e., P]). In some embodiments, the linker can be natural or non-natural amino acid or polypeptide. In other embodiments, the linker is a synthetic compound containing two or more appropriate functional groups suitable to bind, e.g., the CPP and, independently, P. In some embodiments, the linker is about 3 to about 100 (e.g., about 3 to about 20) atoms in linear length (not counting the branched atoms or substituents). In some embodiments, the linker provides about 1 Å to about 400 Å in distance of the two groups to which it connects.

As used herein, “polypeptide” refers to a string of at least two amino acids attached to one another by a peptide bond. There is no upper limit to the number of amino acids that can be included in a polypeptide. Further, polypeptides may include non-natural amino acids, amino acid analogs, or other synthetic molecules that are capable of integrating into a polypeptide.

As used herein, “polyarginine peptide” refers to a string of at least two arginine amino acids (independently D or L). In some embodiments, the polyarginine peptide has the following repeating units where n is an integer selected from 1 to 100 and the polyarginine peptide can have one additional arginine monomer to make an odd-numbered chain.

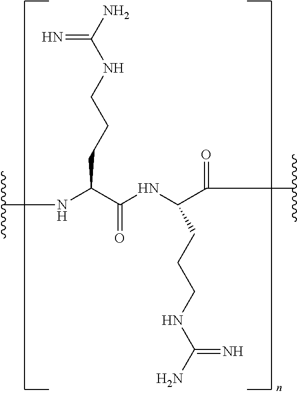

As used herein, a “monomer” refers to an amino acid residue in a polypeptide. In some embodiments, an amino acid monomer is divalent. In other embodiments, an amino acid monomer may be trivalent if the monomer is further substituted. For example, a cysteine monomer can independently form peptide bonds at the N and C termini, and also form a disulfide bond.

As used herein, an “amino acid-analog” or “analog” (e.g., “arginine-analog”, “lysine-analog” or “histidine-analog”) refers to a variant of an amino acid that retains at least one function of the amino acid, such as the ability to bind an oligonucleotide through electrostatic interactions. Such variants may have an elongated or shorter side chain (e.g., by one or more —CH₂— groups that retains the ability to bind an oligonucleotide through electrostatic interactions, or alternatively, the modification can improve the ability to bind an oligonucleotide through electrostatic interactions. For example, an arginine analog may include an additional methylene or ethylene between the backbone and guanidine/guanidinium group. Other examples include amino acids with one or more additional substituents (e.g., Me, Et, halogen, thiol, methoxy, ethoxy, C1-haloalkyl, C2-haloalkyl, amine, guanidine, etc). The amino acid-analog can be monovalent, divalent, or trivalent.

Throughout the present specification, peptides and amino acid monomers are depicted as charge neutral species. It is to be understood that such species may bear a positive or negative charge depending on the conditions. For example, at pH 7, the N-terminus of an amino acid is protonated and bears a positive charge (—NH₃⁺), and the C-terminus of an amino acid is deprotonated and bears a negative charge (—CO₂⁻). Similarly, the side chains of certain amino acids may bear a positive or negative charge.

As used herein, a “charged species” refers to a moiety bearing either a positive or negative charge. For example, when the CPP or cCPP contains arginine or arginine analog monomers, the guanidine group

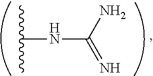

under certain conditions, can be protonated to form a guanidinium group

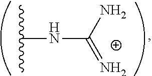

i.e., the charged species. In some embodiments, the between about 5% and about 100% of the moieties in the conjugates described herein that are capable of bearing a charge, are changed, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween.

As used herein, “divalent” refers to moiety having two points of attachment to the rest of the molecule. For example, a divalent linker group was two points of attachment in a polypeptide conjugate, the first point of attachment is to the cell-penetrating peptide (CPP) and the second point of attachment is to the group that binds to a nucleic acid sequence by electrostatic interactions (P).

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₈alkyl. A C₁-C₈alkyl includes C₅ alkyls, C₄ alkyls, C₃ alkyls, C₂ alkyls and C₁ alkyl (i.e., methyl). A C₁-C₆ alkyl includes all moieties described above for C₁-C₈alkyls but also includes C₆ alkyls. A C₁-C₁₀ alkyl includes all moieties described above for C₁-C₈alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, 1-butyl, n-pentyl, 1-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, having from one to forty carbon atoms. Non-limiting examples of C₂-C₄₀ alkylene include ethylene, propylene, n-butylene, pentylene, and the like. Unless stated otherwise specifically in the specification, an alkylene chain can be optionally substituted as described herein.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to forty carbon atoms, and having one or more carbon-carbon double bonds. Non-limiting examples of C₂-C₄₀ alkenylene include ethenylene (—CH═CH—), propenylene, butenylene, and the like. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to forty carbon atoms, and having one or more carbon-carbon triple bonds. Non-limiting examples of C₂-C₄₀ alkynylene include ethynylene (—C≡C—), propargylene and the like. Unless stated otherwise specifically in the specification, an alkynylene chain can be optionally substituted.

“Aryl” refers to a hydrocarbon ring system comprising hydrogen, 6 to 40 carbon atoms and at least one aromatic ring. For purposes of this disclosure, the aryl can be a monovalent or a divalent radical (not counting substituents), which can be a monocyclic, bicyclic, tricyclic or tetracyclic ring system, and which can include fused or bridged ring systems. Aryl radicals include, but are not limited to, radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl radical can be divalent when used as a linker or as a part of a linker. Unless stated otherwise specifically in the specification, an aryl group can be optionally substituted.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Carbocyclic rings include aryls and cycloalkyl and rings that are fully unsaturated, partially unsaturated, and fully saturated. In some embodiments, the carbocyclyl can be divalent when used as a linker or as a part of a linker. Unless stated otherwise specifically in the specification, a carbocyclyl group can be optionally substituted.

“Cycloalkyl” refers to a stable non-aromatic monocyclic or polycyclic fully saturated hydrocarbon radical having from 3 to 40 carbon atoms and at least one ring, wherein the ring consists solely of carbon and hydrogen atoms, which can include fused or bridged ring systems. For purposes of this disclosure, the cycloalkyl can be a monovalent or a divalent radical (not counting substituents). Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. In some embodiments, the cycloalkyl radical can be divalent when used as a linker or as a part of a linker. Unless otherwise stated specifically in the specification, a cycloalkyl group can be optionally substituted.

“Cycloalkenyl” refers to a stable non-aromatic monocyclic or polycyclic hydrocarbon radical having from 3 to 40 carbon atoms, at least one ring having, and one or more carbon-carbon double bonds, wherein the ring consists solely of carbon and hydrogen atoms, which can include fused or bridged ring systems. For purposes of this invention, the cycloalkenyl can be a monovalent or a divalent radical (not counting substituents). Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. In some embodiments, the cycloalkenyl radical can be divalent when used as a linker or as a part of a linker. Unless otherwise stated specifically in the specification, a cycloalkenyl group can be optionally substituted.

“Cycloalkynyl” refers to a stable non-aromatic monocyclic or polycyclic hydrocarbon radical having from 3 to 40 carbon atoms, at least one ring, and one or more carbon-carbon triple bonds, wherein the ring consists solely of carbon and hydrogen atoms, which can include fused or bridged ring systems. For purposes of this invention, the cycloalkynyl can be a monovalent or a divalent radical (not counting substituents). Monocyclic cycloalkynyl radicals include, for example, cycloheptynyl, cyclooctynyl, and the like. In some embodiments, the cycloalkynyl radical can be divalent when used as a linker or as a part of a linker. Unless otherwise stated specifically in the specification, a cycloalkynyl group can be optionally substituted.

“Heterocyclyl,” “heterocyclic ring” or “heterocycle” refers to a stable 3- to 20-membered aromatic ring radical which consists of two to twelve carbon atoms and from one to six heteroatoms selected from the group consisting of nitrogen, oxygen and sulfur. For purposes of this invention, the heterocyclyl radical can be a monovalent or a divalent radical (not counting substituents). Heterocyclycl or heterocyclic rings include heteroaryls as defined below. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In some embodiments, the heterocyclyl radical can be divalent when used as a linker or as a part of a linker. Unless stated otherwise specifically in the specification, a heterocyclyl group can be optionally substituted.

“Heteroaryl” refers to a 5- to 20-membered ring system radical comprising hydrogen atoms, one to fourteen carbon atoms, one to six heteroatoms selected from the group consisting of nitrogen, oxygen and sulfur, and at least one aromatic ring. For purposes of this invention, the heteroaryl radical can be a monovalent or a divalent radical (not counting substituents) and can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). In some embodiments, the heteroaryl radical can be divalent when used as a linker or as a part of a linker. Unless stated otherwise specifically in the specification, a heteroaryl group can be optionally substituted.

The term “ether” used herein refers to a straight or branched divalent radical moiety —[(CH₂)ₘ—O—(CH₂)ₙ]_{z}— wherein each of m, n, and z are independently selected from 1 to 40. Examples include, but are not limited to, polyethylene glycol. Unless stated otherwise specifically in the specification, the ether can be optionally substituted.

The term “substituted” used herein means any of the above groups (i.e., alkylene, alkenylene, alkynylene, aryl, carbocyclyl, cycloalkyl, cycloalkenyl, cycloalkynyl, heterocyclyl, heteroaryl, and/or ether) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents. Further, those skilled in the art will recognize that “substituted” also encompasses instances in which one or more atoms on any of the above groups are replaced by a substituent listed in this paragraph, and the substituent forms a covalent bond with the CPP, P, or L. For example, in certain embodiments, any of the above groups can be substituted at a first position with a carboxylic acid (i.e., —C(═O)OH) which forms an amide bond with a lysine in the CPP, or a group can be substituted at a second position with a thiol group which forms a disulfide bond with a cysteine (or amino acid analog having a thiol group).

### Polypeptide Conjugates

Nucleic acid delivery systems can be divided into two main strategies: 1) viral delivery and 2) non-viral delivery. Viral vectors have the advantage of high efficacy, but can result in immunogenicity and tumorigenicity. Additionally, viral delivery is limited to biologically synthesized nucleic acids and incompatible with short synthetic oligonucleotides or their analogs. Non-viral delivery vectors include various cationic lipids, polymers, carbohydrate analogs, and cell-penetrating peptides (CPPs). These vectors are usually mixed with nucleic acids to form complexes such as nanoparticles or liposomes. The complexes/conjugates are taken up by cells through various endocytic pathways, including macropinocytosis, clathrin- and caveolae-mediated endocytosis. The main limitation of the non-viral delivery systems has been the poor endosomal escape efficiency, resulting in the entrapment of the vast majority of the cargoes inside the endosomal/lysosomal compartments. The nanoparticle-based systems are also limited to distribution into organs/tissues with good blood access and/or relatively large blood vessel fenestrations, such as liver, spleen, or kidney.

The present disclosure relates to a discovery that some CPPs, including cyclic CPPs (cCPPs), are highly active with cytosolic delivery efficiencies of up to 120% (compared to 2% for Tat). The CPPs bind directly to the plasma membrane phospholipids and are internalized by various endocytic mechanisms. The inventors discovered that cCPPs are especially remarkably efficient in endosomal escape, by binding to the early endosomal membrane and inducing budding of small vesicles enriched with the CPPs from the endosomal membrane. Subsequent collapse of the budded vesicles releases the CPPs (and CPP-cargo conjugates) into the cytosol. Moreover, the cCPPs have proven highly effective for cytosolic delivery of a wide variety of cargos, including small molecules, linear peptides, cyclic peptides, and proteins.

The present disclosure relates to a discovery of novel biodegradable polypeptide conjugate comprising at least one CPP, which form non-covalent complexes with nucleic acids (e.g., siRNA). Without being bound by theory, the polypeptide conjugate/nucleic acid complex can effectively enter the cytosol of mammalian cells, where the complex undergoes spontaneous degradation in the reducing environment, releasing the nucleic acid cargo for modulation of the biological activity of a specific target(s).

In various embodiments, the polypeptide conjugates described herein comprise:
a) a group that binds to a nucleic acid sequence by electrostatic interactions (P) comprising at least one peptide or polyamine; and
b) at least one cell-penetrating peptide (CPP);
wherein each peptide comprises at least three monomers selected from arginine, arginine-analog, lysine, lysine-analog, histidine, or histidine-analog; wherein the P is conjugated to the CPP through a bond or at least one linker (L); and
wherein the polypeptide conjugate is optionally charged.

In some embodiments, the polypeptide conjugate as disclosed herein has a molar ratio of P:CPP ranging from about 30:1 to about 1:2. In some embodiments, the molar ratio of P:CPP is about 30:1, about 29:1, about 28:1, about 27:1, about 26:1, about 25:1, about 24:1, about 23:1, about 22:1, about 21:1, about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, or about 1:2, inclusive of all values and subranges therebetween.

In some embodiments, the polypeptide conjugate as disclosed herein has an average molecular weight ranging from about 1 kDa to about 100 kDa. In some embodiments, the average molecular weight of the polypeptide conjugate is about 1 kDa, about 2 kDa, about 3 kDa, about 4 kDa, about 5 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, about 20 kDa, about 21 kDa, about 22 kDa, about 23 kDa, about 24 kDa, about 25 kDa, about 26 kDa, about 27 kDa, about 28 kDa, about 29 kDa, about 30 kDa, about 31 kDa, about 32 kDa, about 33 kDa, about 34 kDa, about 35 kDa, about 36 kDa, about 37 kDa, about 38 kDa, about 39 kDa, about 40 kDa, about 41 kDa, about 42 kDa, about 43 kDa, about 44 kDa, about 45 kDa, about 46 kDa, about 47 kDa, about 48 kDa, about 49 kDa, about 50 kDa, about 51 kDa, about 52 kDa, about 53 kDa, about 54 kDa, about 55 kDa, about 56 kDa, about 57 kDa, about 58 kDa, about 59 kDa, about 60 kDa, about 61 kDa, about 62 kDa, about 63 kDa, about 64 kDa, about 65 kDa, about 66 kDa, about 67 kDa, about 68 kDa, about 69 kDa, about 70 kDa, about 71 kDa, about 72 kDa, about 73 kDa, about 74 kDa, about 75 kDa, about 76 kDa, about 77 kDa, about 78 kDa, about 79 kDa, about 80 kDa, about 81 kDa, about 82 kDa, about 83 kDa, about 84 kDa, about 85 kDa, about 86 kDa, about 87 kDa, about 88 kDa, about 89 kDa, about 90 kDa, about 91 kDa, about 92 kDa, about 93 kDa, about 94 kDa, about 95 kDa, about 96 kDa, about 97 kDa, about 98 kDa, about 99 kDa, or about 100 kDa, inclusive of all ranges and subranges therebetween. In some embodiments, the polypeptide conjugate as disclosed herein has an average molecular weight ranging from about 3 kDa to about 100 kDa.

In some embodiments, the polypeptide conjugate as disclosed herein has the following structure:

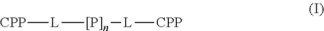

wherein n is an integer from 1 to 50; and
wherein P at each occurrence is same or different.

In some embodiments of the polypeptide conjugate as disclosed herein, P comprises a polyarginine peptide (pArg) comprising at least three monomers selected from arginine or arginine-analog.

In some embodiments of the polypeptide conjugate as disclosed herein, P comprises a polyamine selected from a spermidine polymer or a spermine polymer.

In some embodiments of the polypeptide conjugate as disclosed herein, at least one of the CPP is a cyclic CPP (cCPP). In some embodiments, the CPP is, each independently, a cyclic CPP (cCPP). In some embodiments, cCPP comprises from 4 to 14 amino acid monomers. In other embodiments, cCPP is, each independently, selected from Table 4.

In some embodiments of the polypeptide conjugate as disclosed herein, the cCPP is a cyclo(fΦRrRrQ) peptide or a cyclo(FfΦRrRrQ), wherein:
F is a L-phenylalanine;
f is a D-phenylalanine;
Φ is an L-2-naphthylalanine;
R is a L-arginine;
r is a D-arginine; and
Q is a L-glutamine.

In some embodiments of the polypeptide conjugate as disclosed herein, the pArg comprises at least five arginine monomers or arginine-analog monomers. In some embodiments, the pArg further comprises at least one cysteine monomer.

In some embodiments, the polypeptide conjugate as disclosed herein has the following structure:

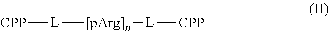

wherein the [pArg]ₙ is

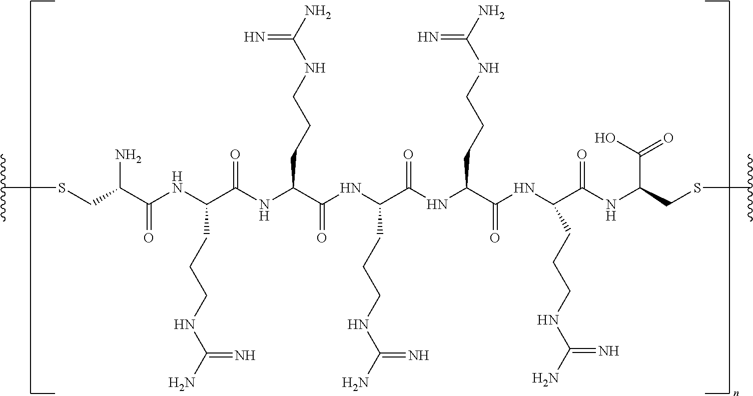

or a charged species thereof.

In some embodiments of the polypeptide conjugate as disclosed herein, n is an integer 1 to 40. In some embodiments of the polypeptide conjugate as disclosed herein, n is an integer 2 to 40. In other embodiments of the polypeptide conjugate as disclosed herein, n is an integer selected from 2 to 30. In other embodiments of the polypeptide conjugate as disclosed herein, n is an integer selected from 2 to 20. In other embodiments of the polypeptide conjugate as disclosed herein, n is an integer selected from 2 to 10.

In some embodiments of the polypeptide conjugate as disclosed herein, at least one L comprises a divalent optionally substituted group selected from amino acid, alkylene, alkenylene, alkynylene, aryl, cycloalkyl, cycloalkenyl, cycloalkynyl, heterocyclyl, heteroaryl, —(R¹—X—R²)z-, or combinations thereof; wherein
each of R¹ and R² are independently selected from a bond, alkylene, alkenylene, alkynylene, carbocyclyl, and heterocyclyl, wherein R¹ and R² are not both a bond;
each X is independently N, S, and O; and
z is an integer selected from 1 to 20.

In some embodiments of the polypeptide conjugate as disclosed herein, at least one L comprises an optionally substituted —(O—CH₂CH₂)z- or an optionally substituted —(CH₂CH₂—O)z-. In some embodiments, at least one L comprises a divalent 8-amino-3,6-dioxaoctanoic acid residue. In other embodiments, at least one L comprises a divalent 8-amino-3,6,9-trioxaundecanoic acid residue.

In some embodiments of the polypeptide conjugate as disclosed herein, at least one L comprises a physiological cleavable group (PCG). In some embodiments, each PCG is, independently, selected from —S—S—, carbonate, thiocarbonate, thioester, sulfoxide, hydrazine, or protease-cleavable dipeptide linker. In other embodiments, each PCG comprises at least one —S—S—.

In some embodiments of the polypeptide conjugate of formula (I) as disclosed herein, at least one of the “-” between L and [P]ₙ represents a bond between two sulfur atoms (disulfide bond). In other embodiments, the “-” between L and [P]ₙ each represents a bond between two sulfur atoms (disulfide bond).

In some embodiments of the polypeptide conjugate as disclosed herein, each P, independently, further comprises at least one group selected from:

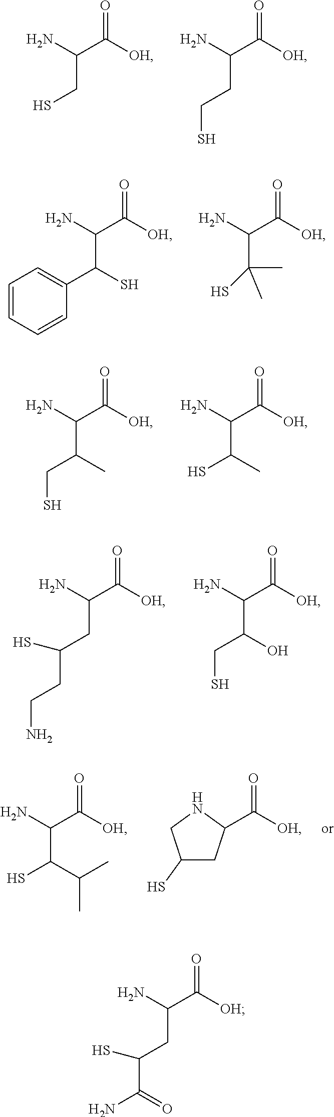

wherein the bond to the hydrogen on at least one of the N- or C-termini is replaced by a bond to the peptide or polyamine; and
wherein the bond to the hydrogen on the thiol group is replaced by a bond to the CPP.

In some embodiments of the polypeptide conjugate as disclosed herein, each P, independently, further comprises at least one group selected from:

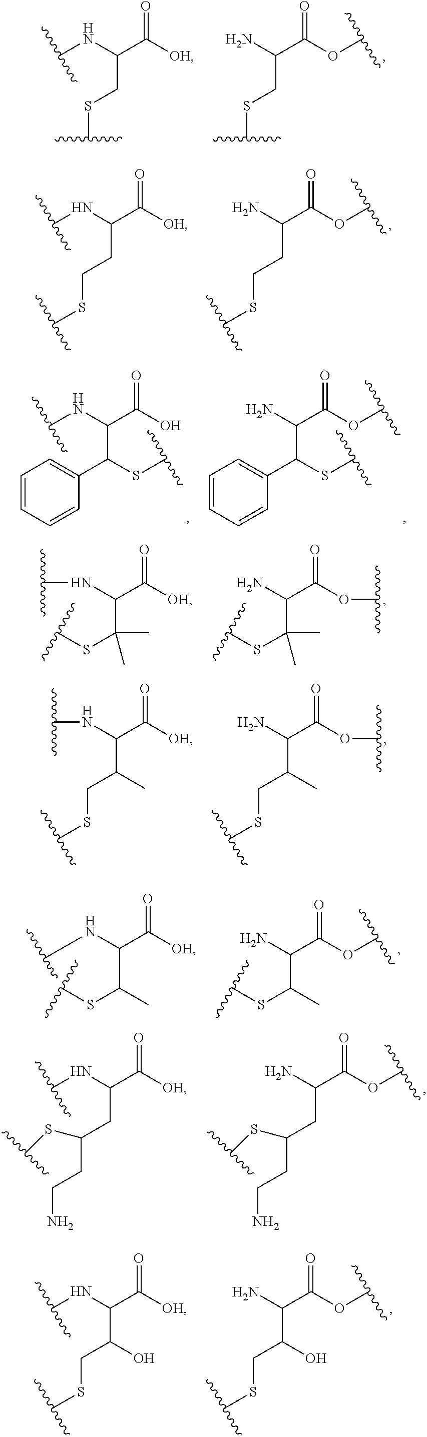

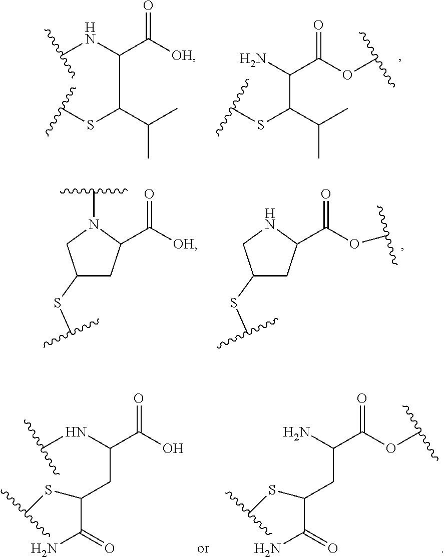

In some embodiments of the polypeptide conjugate as disclosed herein, at least one of the P further comprises

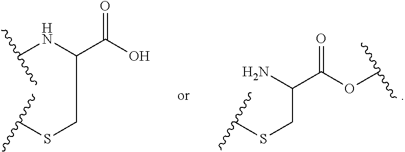 [SMILES: *NC(CS*)C(=O)O ; *OC(=O)C(N)CS*]

In other embodiments, at least one of the P further comprises at least two groups selected from

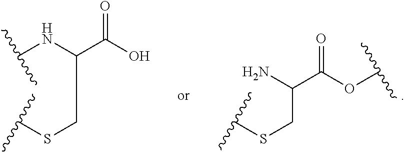 [SMILES: *NC(CS*)C(=O)O ; *OC(=O)C(N)CS*]

In some embodiments, the polypeptide conjugate as disclosed herein has the following structure:

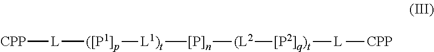

wherein
n, p, and q are each independently an integer from 1 to 50;
t is each independently 0 or 1;
P¹ and P² each comprises at least one peptide or polyamine, wherein each peptide comprises at least three monomers selected from arginine, arginine-analog, lysine, lysine-analog, histidine, or histidine-analog, wherein P, P¹ and P², at each occurrence, are same or different; and
L¹ and L² are each independently absent or L as defined in claim 1, wherein L, L¹, and L², at each occurrence, are same or different.

In some embodiments of the polypeptide conjugate of formula (III) as disclosed herein, P, P¹ or P² comprises a polyarginine peptide (pArg) comprising at least three monomers selected from arginine or arginine-analog. In some embodiments of the polypeptide conjugate of formula (III) as disclosed herein, P, P¹ or P² comprises a polyarginine peptide (pArg) comprising three monomers selected from arginine or arginine-analog. In other embodiments, P, P¹ or P² comprises a polyamine selected from a spermidine polymer or a spermine polymer.

In some embodiments of the polypeptide conjugate of formula (III) as disclosed herein, P, P¹ or P² comprises mixtures of monomers selected from arginine, arginine-analog, lysine, lysine-analog, histidine, or histidine-analog (e.g., -Arg-Lys-Arg-; -Arg-Arg-His-; etc). In some embodiments of the polypeptide conjugate of formula (III) as disclosed herein, P, P¹ or P² further comprises at least one amino acid monomer (e.g., such as those shown in Table 1).

In some embodiments of the polypeptide conjugate of formula (III) as disclosed herein, at least one of the CPP is a cyclic CPP (cCPP). In other embodiments, the CPP is, each independently, a cyclic CPP (cCPP). In one embodiment, the cCPP comprises from 4 to 14 amino acid monomers. In another embodiment, the cCPP is, each independently, selected from Table 4.

In some embodiments of the polypeptide conjugate of formula (III), the cCPP is a cyclo(fΦRrRrQ) (SEQ ID NO: 118) peptide or a cyclo(FfΦRrRrQ) (SEQ ID NO: 16), wherein:
F is a L-phenylalanine;
f is a D-phenylalanine;
Φ is an L-2-naphthylalanine;
R is a L-arginine;
r is a D-arginine; and
Q is a L-glutamine.

In some embodiments of the polypeptide conjugate of formula (III), the pArg comprises at least three arginine monomers or arginine-analog monomers. In some embodiments of the polypeptide conjugate of formula (III), the pArg comprises three arginine monomers or arginine-analog monomers. In some embodiments of the polypeptide conjugate of formula (III), the pArg comprises at least five arginine monomers or arginine-analog monomers. In other embodiments, the pArg further comprises at least one amino acid monomer (e.g., such as those shown in Table 4). In one embodiment, the pArg further comprises at least one cysteine monomer. In one embodiment, at least one pArg further comprises a thioether moiety (—S—).

In some embodiments of the polypeptide conjugate of formula (III), n is an integer 1 to 40. In some embodiments of the polypeptide conjugate of formula (III), n is an integer 5 to 40.

In some embodiments of the polypeptide conjugate of formula (III), at least one of L, L¹, or L² comprises a divalent optionally substituted group selected from amino acid, polyethylene glycol, alkylene, alkenylene, alkynylene, aryl, cycloalkyl, cycloalkenyl, cycloalkynyl, heterocyclyl, heteroaryl, —(R¹—X—R²)z-, or combinations thereof;
each of R1 and R2 are independently selected from a bond, alkylene, alkenylene, alkynylene, carbocyclyl, and heterocyclyl, wherein R1 and R2 are not both a bond;
each X is independently N, S, and O; and
z is an integer selected from 1 to 20.

In some embodiments of the polypeptide conjugate of formula (III), at least one of L, L¹, or L² comprises an optionally substituted —(O—CH₂CH₂)z- or an optionally substituted —(CH₂CH₂—O)z-.

In some embodiments of the polypeptide conjugate of formula (III), at least one of L, L¹, or L² comprises a divalent 8-amino-3,6-dioxaoctanoic acid residue. In some embodiments, at least one of L, L¹, or L² comprises a divalent 8-amino-3,6,9-trioxaundecanoic acid residue. In other embodiments, at least one of L, L¹, or L² comprises a physiological cleavable group (PCG).

In some embodiments of the polypeptide conjugate of formula (III), each PCG is, independently, selected from —S—S—, carbonate, thiocarbonate, thioester, sulfoxide, hydrazine, or protease-cleavable dipeptide linker. In some embodiments, each PCG comprises at least one —S—S—.

In some embodiments of the polypeptide conjugate of formula (III), at least one of the “-” between L and ([P¹]ₚ-L¹)ₜ, L and [P]ₙ, or L and (L²-[P²]_{q})ₜ represents a bond between two sulfur atoms (disulfide bond).

In some embodiments of the polypeptide conjugate of formula (III), at least one of P, P¹ or P² further comprises at least one group selected from:

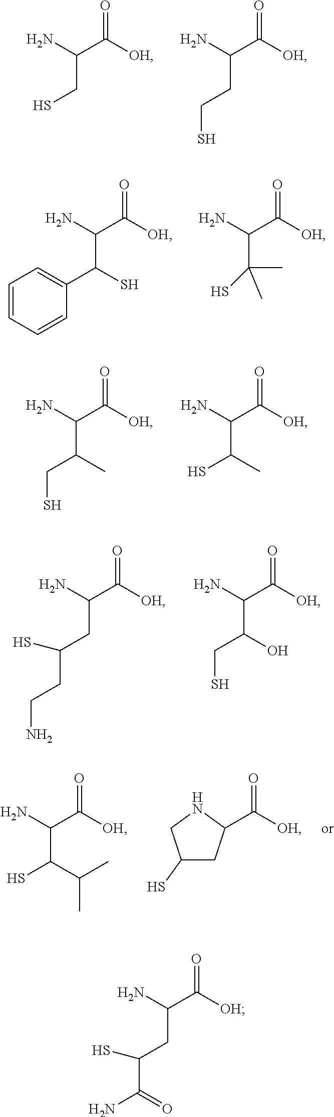

wherein the bond to the hydrogen on at least one of the N- or C-termini is replaced by a bond to the peptide or polyamine; and
wherein the bond to the hydrogen on the thiol group is replaced by a bond to the CPP.

In some embodiments of the polypeptide conjugate of formula (III), at least one of P, P¹ or P³ further comprises at least one group selected from:

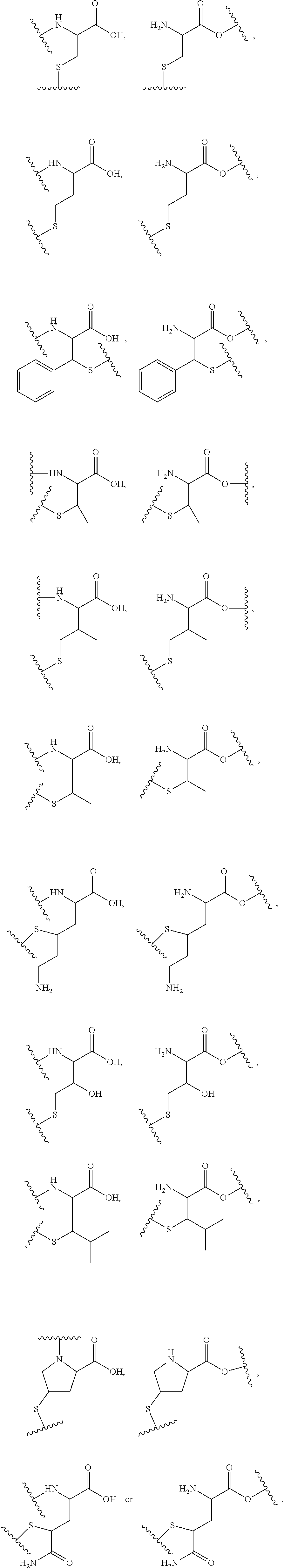

In some embodiments of the polypeptide conjugate of formula (III), at least one of P, P¹ or P² further comprises

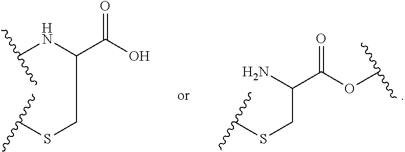 [SMILES: *NC(CS*)C(=O)O ; *OC(=O)C(N)CS*]

In other embodiments, at least at least two of P, P¹ or P² further comprises a group selected form

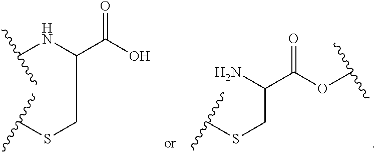 [SMILES: *NC(CS*)C(=O)O ; *OC(=O)C(N)CS*]

In some embodiments of the polypeptide conjugate of formula (III), the pArg further comprises one or more β-alanine monomers. In some embodiments, each pArg comprises, independently, 3, 4, 5, 6, 7, or 8 arginine monomers. In other embodiments, each pArg comprises 3 arginine monomers. In other embodiments, each pArg comprises 5 arginine monomers. In other embodiments, at least one pArg further comprises at least one amino acid monomer (e.g., such as those shown in Table 4). In one embodiment, at least one pArg further comprises a cysteine monomer. In one embodiment, at least one pArg further comprises a thioether moiety (—S—).

In some embodiments of the polypeptide conjugate of formula (III), n, p, and q are each independently an integer from 1 to 40. In other embodiments, n, p, and q are each independently an integer from 1 to 30. In other embodiments, n, p, and q are each independently an integer from 1 to 20. In other embodiments, n, p, and q are each independently an integer from 1 to 10. In other embodiments, n, p, and q are each independently an integer from 1 to 5.

In some embodiments of the polypeptide conjugate of formula (I), (II), or (III) as disclosed herein, P is each independently selected from: -Cys-(Arg)_{X}-Cys- (SEQ ID NO: 131), -Cys-βAla-(Arg)_{X}-Cys- (SEQ ID NO: 132), -Cys-(Arg)_{X}-βAla-Cys- (SEQ ID NO: 133), or -Cys-βAla-(Arg)_{X}-βAla-Cys- (SEQ ID NO: 134), -Cys-(polyamine)_{X}-Cys-, -Cys-βAla-(polyamine)_{X}-Cys-, -Cys-(polyamine)_{X}-βAla-Cys-, -Cys-βAla-(polyamine)_{X}-βAla-Cys-(SEQ ID NO: 135), or —S-(polyamine)_{X}-S—, wherein x=3, 4, 5, 6, 7, or 8.

In some embodiments, the polypeptide conjugate of formula (I) as disclosed herein has the structure

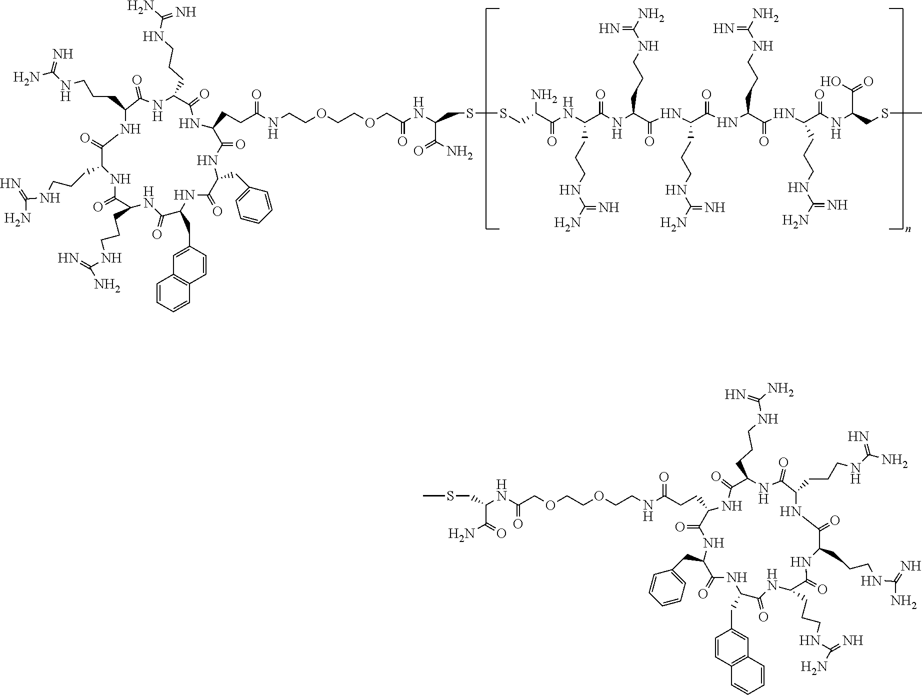

or a charged species thereof.

In some embodiments, the polypeptide conjugate of formula (I) as disclosed herein has the structure

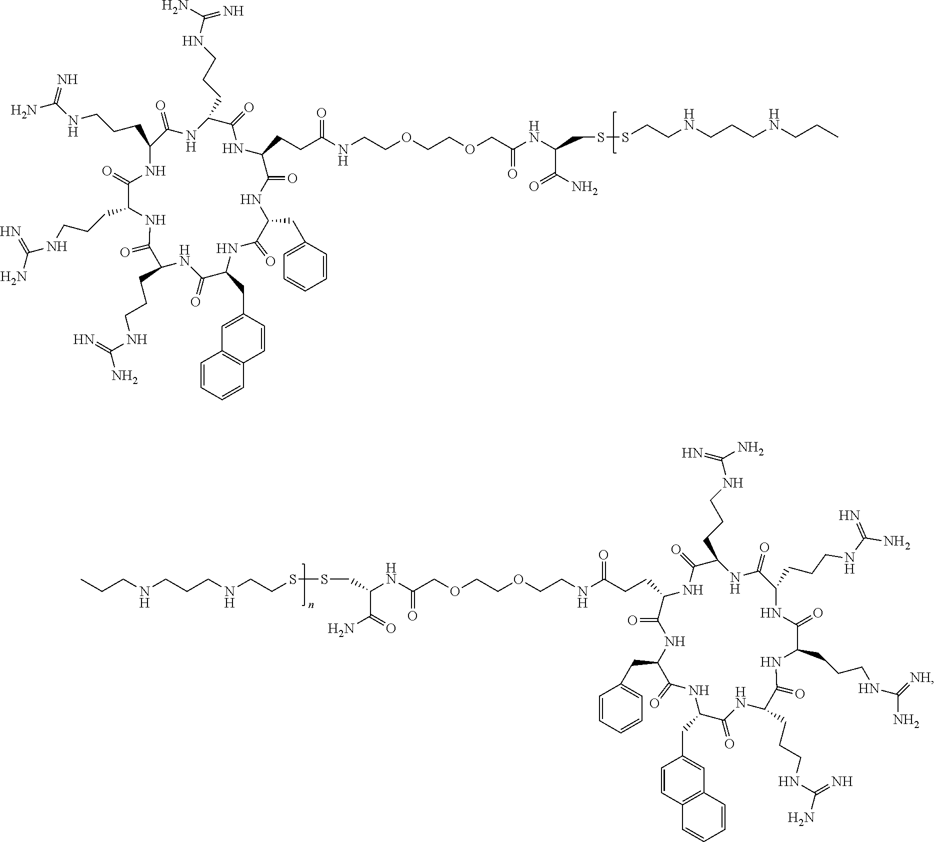

or a charged species thereof.

In some embodiments, the polypeptide conjugate as disclosed herein has the following structure:

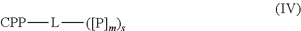

wherein:
s is an integer from 1 to 10;
each m is, independently, an integer from 1 to 50; and
wherein P at each occurrence is same or different.

In some embodiments of the polypeptide conjugate of formula (IV) as disclosed herein, P comprises a polyarginine peptide (pArg) comprising at least three monomers selected from arginine or arginine-analog. In some embodiments, P comprises a polyamine selected from a spermidine polymer or a spermine polymer.

In some embodiments of the polypeptide conjugate of formula (IV) as disclosed herein, at least one of the CPP is a cyclic CPP (cCPP). In some embodiments, the cCPP comprises from 4 to 14 amino acid monomers. In other embodiments, the cCPP is, each independently, selected from Table 4.

In some embodiments of the polypeptide conjugate of formula (IV) as disclosed herein, the pArg comprises three arginine monomers or arginine-analog monomers. In some embodiments of the polypeptide conjugate of formula (IV) as disclosed herein, the pArg comprises at least five arginine monomers or arginine-analog monomers. In other embodiments, the pArg further comprises at least one amino acid monomer (e.g., such as those shown in Table 4). In some embodiments, the pArg further comprises at least one cysteine monomer. In one embodiment, the pArg further comprises a cysteine monomer. In one embodiment, at least one pArg further comprises a thioether moiety (—S—).

In some embodiments of the polypeptide conjugate of formula (IV) as disclosed herein, L comprises a divalent optionally substituted group selected from amino acid, polyethylene glycol, alkylene, alkenylene, alkynylene, aryl, cycloalkyl, cycloalkenyl, cycloalkynyl, heterocyclyl, heteroaryl, —(R¹—X—R²)z-, or combinations thereof;
each of R¹ and R² are independently selected from a bond, alkylene, alkenylene, alkynylene, carbocyclyl, and heterocyclyl, wherein R1 and R2 are not both a bond;
each X is independently N, S, and O; and
z is an integer selected from 1 to 20.

In some embodiments of the polypeptide conjugate of formula (IV) as disclosed herein, L comprises an optionally substituted —(O—CH₂CH₂)z- or an optionally substituted —(CH₂CH₂—O)z-. In some embodiments, L comprises a divalent 8-amino-3,6-dioxaoctanoic acid residue. In other embodiments, L comprises a divalent 8-amino-3,6,9-trioxaundecanoic acid residue. In one embodiment, L comprises a physiological cleavable group (PCG).

In some embodiments of the polypeptide conjugate of formula (IV) as disclosed herein, each PCG is, independently, selected from —S—S—, carbonate, thiocarbonate, thioester, sulfoxide, hydrazine, or protease-cleavable dipeptide linker. In some embodiments, each PCG comprises at least one —S—S—.

In some embodiments of the polypeptide conjugate of formula (IV) as disclosed herein, L comprises a polythiolamine or a 3,5-bis(mercaptomethyl)benzoyl (Bmb) amide.

In some embodiments of the polypeptide conjugate of formula (IV), L comprises two or more physiological cleavable groups.

In some embodiments of the polypeptide conjugate of formula (IV) as disclosed herein, the “-” between L and ([P]ₘ)ₛ represents a bond between two sulfur atoms (disulfide bond).

In some embodiments of the polypeptide conjugate of formula (IV) as disclosed herein, each P, independently, further comprises at least one group selected from:

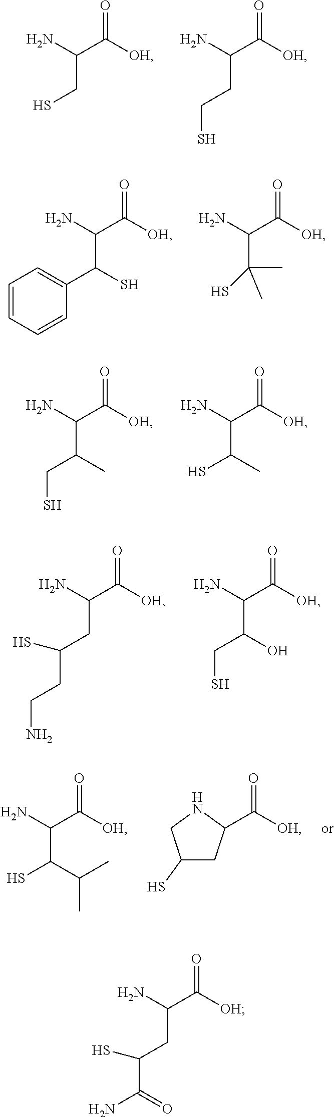

wherein the bond to the hydrogen on one at least one of the N- or C-termini is replaced by a bond to the peptide or polyamine; and
wherein the bond to the hydrogen on the thiol group is replaced by a bond to the CPP.

In some embodiments of the polypeptide conjugate of formula (IV) as disclosed herein, each P, independently, further comprises at least one group selected from:

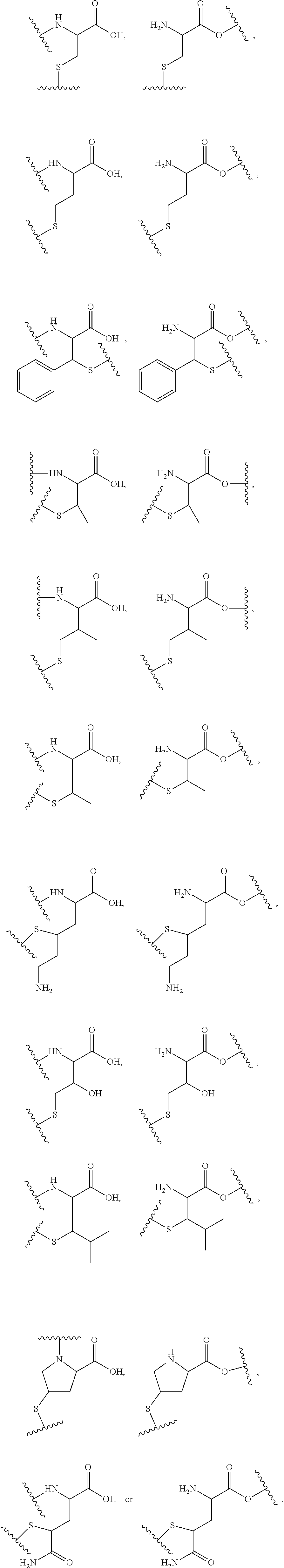

In some embodiments of the polypeptide conjugate of formula (IV) as disclosed herein, at least one of the P further comprises

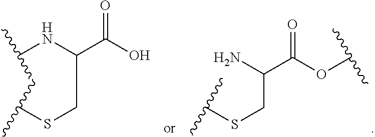 [SMILES: *NC(CS*)C(=O)O ; *OC(=O)C(N)CS*]

In other embodiments, at least one of the P further comprises at least two groups selected form

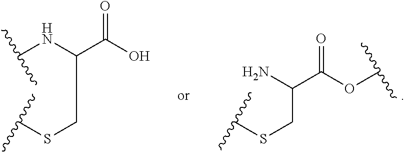 [SMILES: *NC(CS*)C(=O)O ; *OC(=O)C(N)CS*]

In some embodiments of the polypeptide conjugate of formula (IV) as disclosed herein, sis 2, 3, 4, or 5.

In some embodiments, the polypeptide conjugate of formula (IV) as disclosed herein has the structure

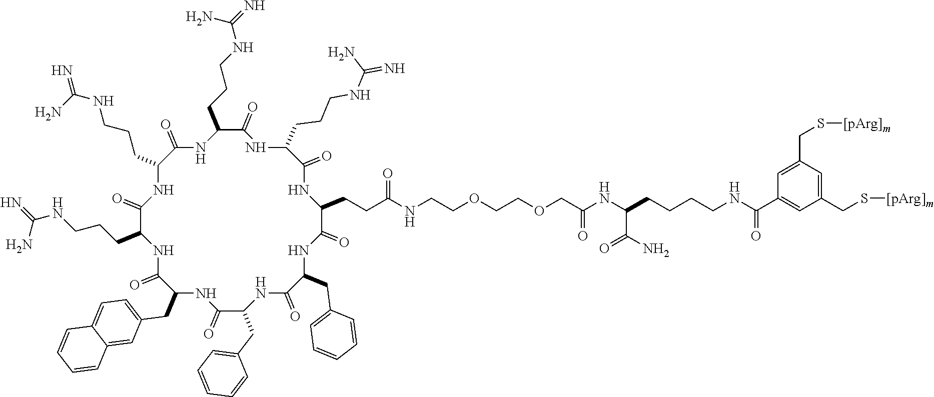

or charged species thereof.

In some embodiments, the polypeptide conjugate of formula (IV) as disclosed herein has the structure

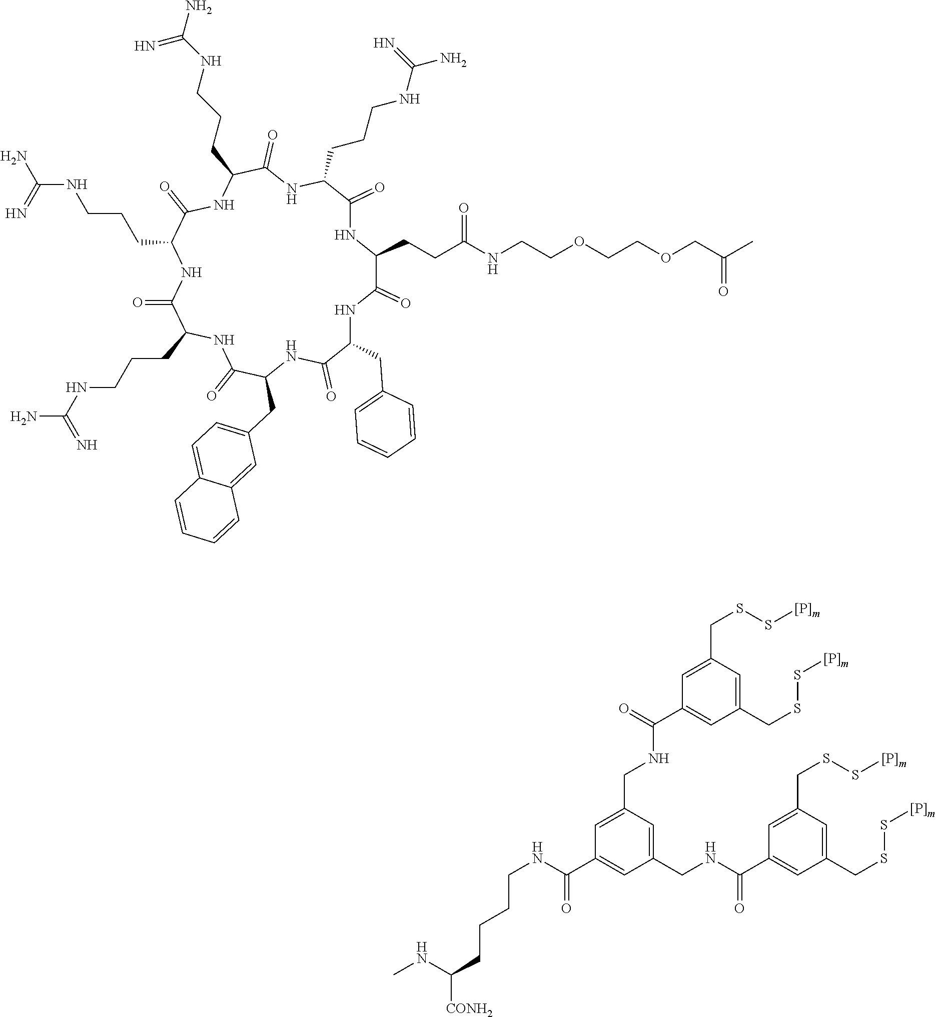

or charged species thereof.

In some embodiments, the polypeptide conjugate as disclosed herein has the following structure:

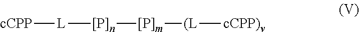

wherein,
cCPP is a cyclic CPP; and
n is an integer selected from 1 to 50;
m is an integer selected from 0 to 49 provided that the sum of n and m is 50 or less;
v is 0 or 1;
wherein P at each occurrence is same or different; and
wherein when v is 0, the last [P] in [P]ₘ is monovalent.

In some embodiments of the polypeptide conjugate of formula (V), v is 1.

In some embodiments of the polypeptide conjugate of formula (V) as disclosed herein, the “-” between [P]ₙ and [P]ₘ represents a bond between two sulfur atoms (a disulfide bond).

In some embodiments, the polypeptide conjugate of formula (IV) as disclosed herein, has the following structure:

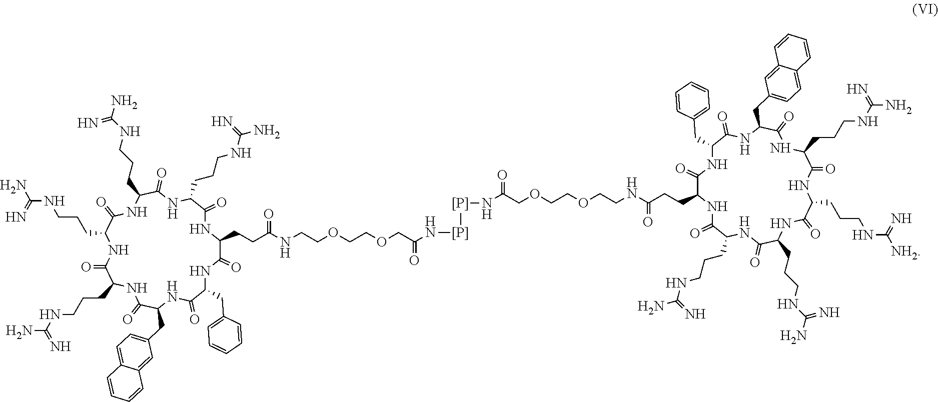

In some embodiments of the polypeptide conjugate of formula (VI) as disclosed herein, the “-” between [P] and [P] represents a bond between two sulfur atoms (a disulfide bond).

In some embodiments, the polypeptide conjugate as disclosed herein has the following structure:

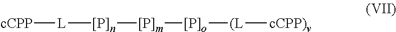

wherein,
cCPP is a cyclic CPP; and
n is an integer selected from 1 to 50;
m and o, are each independently, an integer from 0 to 49 provided that the sum of n, m, and o is 50 or less;
v is 0 or 1;
wherein P at each occurrence is same or different; and
wherein when v is 0, the last [P] in [P]ₒ is monovalent.

In some embodiments of the polypeptide conjugate of formula (VII), v is 1.

In some embodiments of the polypeptide conjugate of formula (VII) as disclosed herein, at least one of the “-” between [P]ₙ and [P]ₘ or [P]ₘ and [P]ₒ represents a bond between two sulfur atoms (a disulfide bond). In other embodiments, the “-” between [P]ₙ and [P]ₘ and between [P]ₘ and [P]ₒ represents a bond between two sulfur atoms (a disulfide bond).

In some embodiments, the polypeptide conjugate of formula (VI) as disclosed herein, has the following structure:

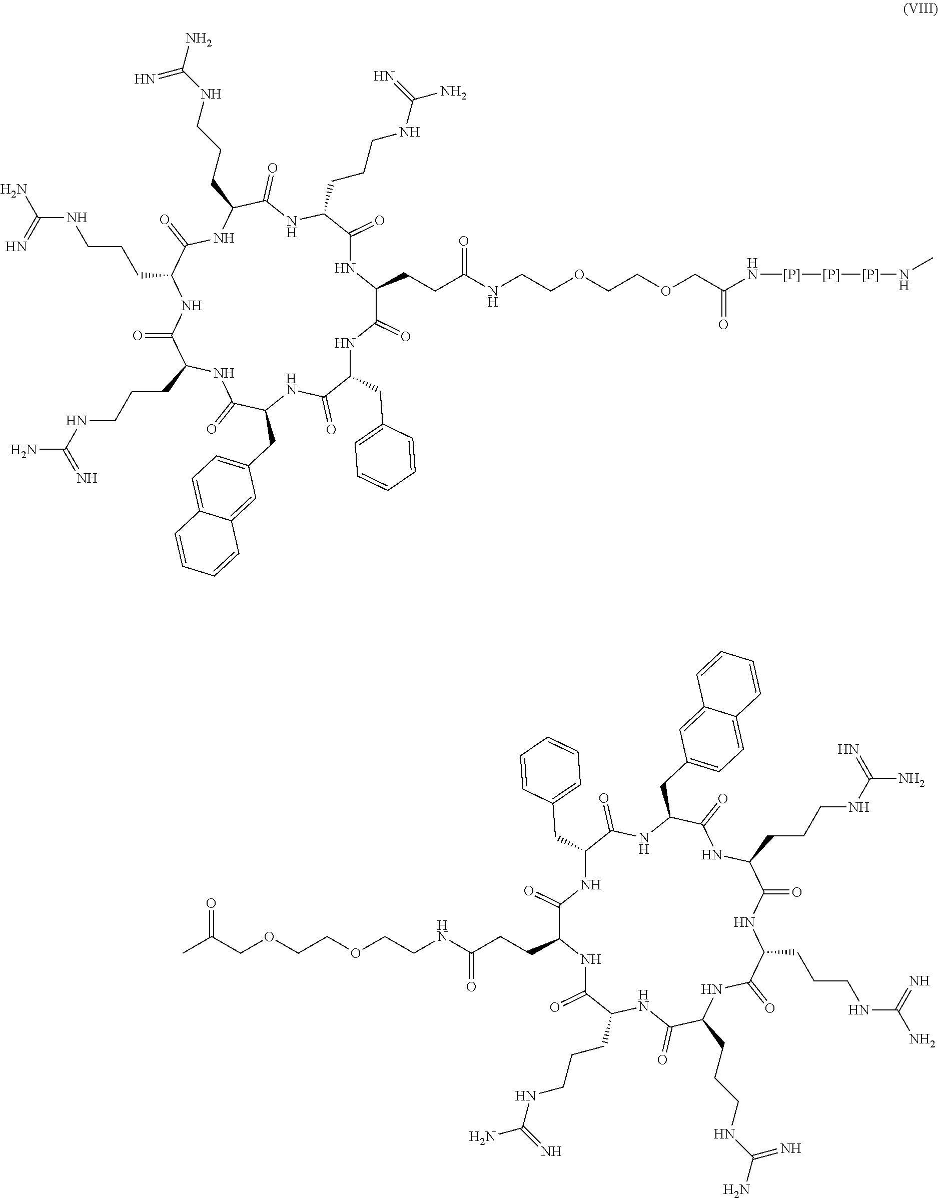

In some embodiments of the polypeptide conjugate of formula (VIII) as disclosed herein, at least one of the “-” between [P] and [P] represents a bond between two sulfur atoms (a disulfide bond). In other embodiment, each “-” between [P] and [P] represents a bond between two sulfur atoms (a disulfide bond).

In some embodiment, the dash “-” between P (including P¹ and P²), L (including L¹ and L²), and CPP (including cCPP) in formula (I), (II), (III), (IV), (V), (VI), (VII), and (VIII) represents each component's spatial orientation. To be clear, “-” not strictly as a C—C bond, although in some embodiments, it may be. In some embodiments, each P can be discussed as its own component having a chemical group necessary to covalently attach to L. In some embodiments, each P can be discussed as its own component having a chemical group necessary to covalently attach to another group of P. In some embodiments, the CPP can be discussed as its own component having a chemical group necessary to covalently attach to L. One skilled in the art would readily understand how each component, described separately, can covalently attach to one another to provide the polypeptide conjugate as disclosed herein.

In some embodiments, any of L (including L¹ and L²) disclosed herein can be the linker that is covalently attached to P and/or to the CPP. In some embodiments, any of L disclosed herein can describe the linker moiety before covalently attaching it to P and/or to CPP. In a non-limited example, L can comprise a chemical group (e.g., —SH, —NH₂, —OH etc) which can be reacted with another chemical group on or attached to P or CPP in order to form a covalent bond, e.g., disulfide bond (—S—S—), amine bond (—NH—), ether bond (—O—), amide bond (—NH(O)—), ester bond (—C(O)O—), etc. In one embodiment, a chemical group already present in L as described herein can be used to covalently attach L to the P and/or to the CPP. The chemistry used to covalently attach P to L and L to CPP can be readily understood by one skilled in the art.

In one embodiment, any of L disclosed herein can further comprise a chemical group useful in covalently attaching L to the P and/or to the CPP.

### Cell-Penetrating Peptides (CPP)

As discussed above, the polypeptide conjugates disclosed herein comprise cell-penetrating peptides (CPPs). CPPs are peptides that facilitate cellular intake or uptake of various molecular equipment, often refers to as a “cargo” (e.g., nucleic acid or siRNA). The “cargo” is associated with the peptides through non-covalent interactions, such as by electrostatic interactions. The function of the CPPs are to deliver the cargo into cells, a process that commonly occurs through endocytosis and subsequently released into cytosol of mammalian cells.

(i) CPPs typically have an amino acid composition that either contains a high relative abundance of positively charged amino acids such as arginine or has sequences that contain an alternating pattern of polar/charged amino acids and non-polar, hydrophobic amino acids. Some CPPs comprise hydrophobic peptides, containing only apolar residues, with low net charge or have hydrophobic amino acid groups that are crucial for cellular uptake. Non-limiting examples of linear CPPs include Polyarginine (e.g., R₉ or R₁₁), Antennapedia sequences, HIV-TAT, Penetratin, Antp-3A (Antp mutant), Buforin II. Transportan, MAP (model amphipathic peptide), K-FGF, Ku70, Prion, pVEC, Pep-1, SynB1, Pep-7, HN-1, BGSC (Bis-Guanidinium-Spermidine-Cholesterol, and BGTC (Bis-Guanidinium-Tren-Cholesterol).

In some embodiments, CPPs are cyclic CPPs (cCPPs). The cCPP may be or include any amino sequence, which facilitates cellular uptake of the polypeptide conjugates disclosed herein. Suitable cCPPs for use in the polypeptide conjugates and methods described herein can include naturally occurring sequences, modified sequences, and synthetic sequences. In embodiments, the total number of amino acids in the cCPP may be in the range of from 4 to about 20 amino acids, e.g., about 5, about 6, about 7, about 8, about 9, about 10, about 11, about 12, about 13, about 14, about 15, about 16, about 17, about 18, and about 19 amino acids, inclusive of all ranges and subranges therebetween. In some embodiments, the cCPPs disclosed herein comprise about 4 to about to about 13 amino acids. In particular embodiments, the CPPs disclosed herein comprise about 6 to about 10 amino acids, or about 6 to about 8 amino acids.

Each amino acid in the CPP or cCPP may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be the D-isomer of the natural amino acids. Examples of suitable amino acids include, but are not limited to, alanine, allosoleucine, arginine, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative, or combinations thereof. These, and others, are listed in the Table 1 along with their abbreviations used herein.

TABLE 1
Amino Acid Abbreviations

| | Abbreviations* | Abbreviations* |
|---|---|---|
| Amino Acid | L-amino acid | D-amino acid |
| Alanine | Ala (A) | ala (a) |
| Allosoleucine | AIle | aile |
| Arginine | Arg (R) | arg (r) |
| Asparagine | Asn (N) | asn (n) |
| aspartic acid | Asp (D) | asp (d) |
| Cysteine | Cys (C) | cys (c) |
| Cyclohexylalanine | Cha | cha |
| 2,3-diaminopropionic acid | Dap | dap |
| 4-fluorophenylalanine | Fpa (Σ) | pfa |
| glutamic acid | Glu (E) | glu (e) |
| glutamine | Gln (Q) | gln (q) |
| glycine | Gly (G) | gly (g) |
| histidine | His (H) | his (h) |
| Homoproline (aka pipecolic acid) | Pip (Θ) | pip (θ) |
| isoleucine | Ile (I) | ile (i) |
| leucine | Leu (L) | leu (l) |
| lysine | Lys (K) | lys (k) |
| methionine | Met (M) | met (m) |
| napthylalanine | Nal (Φ) | nal (φ) |
| norleucine | Nle (Ω) | nle |
| phenylalanine | Phe (F) | phe (F) |
| phenylglycine | Phg (Ψ) | phg |
| 4-(phosphonodifluoromethyl) | F₂Pmp (Λ) | f₂pmp |
| phenylalanine | | |
| proline | Pro (P) | pro (p) |
| sarcosine | Sar (Ξ) | sar |
| selenocysteine | Sec (U) | sec (u) |
| serine | Ser (S) | ser (s) |
| threonine | Thr (T) | thr (y) |
| tyrosine | Tyr (Y) | tyr (y) |
| tryptophan | Trp (W) | trp (w) |
| valine | Val (V) | val (v) |

*single letter abbreviations: when shown in capital letters herein it indicates the L-amino acid form, when shown in lower case herein it indicates the D-amino acid form.

In some embodiments, the cCPPs may include any combination of at least two arginines and at least two hydrophobic amino acids. In some embodiments, the cCPPs may include any combination of two to three arginines and at least two hydrophobic amino acids.

In some embodiments, the CPP used in polypeptide conjugates described herein has a structure comprising Formula 1:

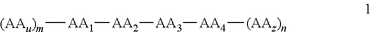

wherein:
each of AA₁, AA₂, AA₃, and AA₄, are independently selected from a D or L amino acid,
each of AAᵤ and AA_{z}, at each instance and when present, are independently selected from a D or L amino acid, and
m and n are independently selected from a number from 0 to 6.

In some embodiments of the Formula 1, at least two of AAᵤ (when present), AA₁, AA₂, AA₃, AA₄, and AA_{z} (when present), are independently arginine. In some embodiments, at least two of AAᵤ (when present), AA₁, AA₂, AA₃, AA₄, and AA_{z} (when present), are independently an amino acid having a hydrophobic side chain. In some embodiments, at least two of AAᵤ (when present), AA₁, AA₂, AA₃, AA₄, and AA_{z} (when present), are independently arginine and at least two of AAᵤ (when present), AA₁, AA₂, AA₃, AA₄, and AA_{z} (when present), are independently an having a hydrophobic side chain.

In some embodiments, each amino acid having a hydrophobic side chain is independently selected from glycine, alanine, valine, leucine, isoleucine, methionine, phenylalanine, tryptophan, proline, naphthylalanine, phenylglycine, homophenylalanine, tyrosine, cyclohexylalanine, piperidine-2-carboxylic acid, or norleucine, each of which is optionally substituted with one or more substituents. In particular embodiments, each amino acid having a hydrophobic side chain is independently an amino acid having a hydrophobic aromatic side chain. In some embodiments, the amino acid having a hydrophobic aromatic side chain is naphthylalanine, phenylglycine, homophenylalanine, phenylalanine, tryptophan, or tyrosine, each of which is optionally substituted with one or more substituents. In particular embodiments, the amino acid having a hydrophobic side chain is piperidine-2-carboxylic acid, naphthylalanine, tryptophan, or phenylalanine, each of which is optionally substituted with one or more substituents.

The optional substituent can be any atom or group which does not significantly reduce the cytosolic delivery efficiency of the CPP or cCPP, e.g., a substituent that does not reduce relative cytosolic delivery efficiency to less than that of c (FΦRRRRQ) (SEQ ID NO: 9). In some embodiments, the optional substituent can be a hydrophobic substituent or a hydrophilic substituent. In certain embodiments, the optional substituent is a hydrophobic substituent. In some embodiments, the substituent increases the solvent-accessible surface area (as defined herein) of the hydrophobic amino acid. In some embodiments, the substituent can be a halogen, alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio. In some embodiments, the substituent is a halogen.

Amino acids having higher hydrophobicity values can be selected to improve cytosolic delivery efficiency of a CPP relative to amino acids having a lower hydrophobicity value. In some embodiments, each hydrophobic amino acid independently has a hydrophobicity value which is greater than that of glycine. In other embodiments, each hydrophobic amino acid independently is a hydrophobic amino acid having a hydrophobicity value which is greater than that of alanine. In still other embodiments, each hydrophobic amino acid independently has a hydrophobicity value which is greater or equal to phenylalanine. Hydrophobicity may be measured using hydrophobicity scales known in the art. Table 2 below lists hydrophobicity values for various amino acids as reported by Eisenberg and Weiss (Proc. Natl. Acad. Sci. U.S.A. 1984; 81(1):140-144), Engleman, et al. (Ann. Rev. of Biophys. Biophys. Chem. 1986; 1986(15):321-53), Kyte and Doolittle (J. Mol. Biol. 1982; 157(1):105-132), Hoop and Woods (Proc. Natl. Acad. Sci. U.S.A. 1981; 78(6):3824-3828), and Janin (Nature. 1979; 277(5696):491-492), the entirety of each of which is herein incorporated by reference in its entirety. In particular embodiments, hydrophobicity is measured using the hydrophobicity scale reported in Engleman, et al.

TABLE 2
Hydrophobicity Values of Amino Acids

| Ami- | | | | Kyrie | Hoop | |
|---|---|---|---|---|---|---|
| no | | Eisenberg | Engleman | and | and | |
| Acid | Group | and Weiss | et al. | Doolittle | Woods | Janin |
| Ile | Nonpolar | 0.73 | 3.1 | 4.5 | −1.8 | 0.7 |
| Phe | Nonpolar | 0.61 | 3.7 | 2.8 | −2.5 | 0.5 |
| Val | Nonpolar | 0.54 | 2.6 | 4.2 | −1.5 | 0.6 |
| Leu | Nonpolar | 0.53 | 2.8 | 3.8 | −1.8 | 0.5 |
| Trp | Nonpolar | 0.37 | 1.9 | −0.9 | −3.4 | 0.3 |
| Met | Nonpolar | 0.26 | 3.4 | 1.9 | −1.3 | 0.4 |
| Ala | Nonpolar | 0.25 | 1.6 | 1.8 | −0.5 | 0.3 |
| Gly | Nonpolar | 0.16 | 1.0 | −0.4 | 0.0 | 0.3 |
| Cys | Unch/Polar | 0.04 | 2.0 | 2.5 | −1.0 | 0.9 |
| Tyr | Unch/Polar | 0.02 | −0.7 | −1.3 | −2.3 | −0.4 |
| Pro | Nonpolar | −0.07 | −0.2 | −1.6 | 0.0 | −0.3 |
| Thr | Unch/Polar | −0.18 | 1.2 | −0.7 | −0.4 | −0.2 |
| Ser | Unch/Polar | −0.26 | 0.6 | −0.8 | 0.3 | −0.1 |
| His | Charged | −0.40 | −3.0 | −3.2 | −0.5 | −0.1 |
| Glu | Charged | −0.62 | −8.2 | −3.5 | 3.0 | −0.7 |
| Asn | Unch/Polar | −0.64 | −4.8 | −3.5 | 0.2 | −0.5 |
| Gln | Unch/Polar | −0.69 | −4.1 | −3.5 | 0.2 | −0.7 |
| Asp | Charged | −0.72 | −9.2 | −3.5 | 3.0 | −0.6 |
| Lys | Charged | −1.10 | −8.8 | −3.9 | 3.0 | −1.8 |
| Arg | Charged | −1.80 | −12.3 | −4.5 | 3.0 | −1.4 |

The chirality of the amino acids can be selected to improve cytosolic uptake efficiency. In some embodiments, at least two of the amino acids have the opposite chirality. In some embodiments, the at least two amino acids having the opposite chirality can be adjacent to each other. In some embodiments, at least three amino acids have alternating stereochemistry relative to each other. In some embodiments, the at least three amino acids having the alternating chirality relative to each other can be adjacent to each other. In some embodiments, at least two of the amino acids have the same chirality. In some embodiments, the at least two amino acids having the same chirality can be adjacent to each other. In some embodiments, at least two amino acids have the same chirality and at least two amino acids have the opposite chirality. In some embodiments, the at least two amino acids having the opposite chirality can be adjacent to the at least two amino acids having the same chirality. Accordingly, in some embodiments, adjacent amino acids in the CPP can have any of the following sequences: D-L; L-D; D-L-L-D; L-D-D-L; L-D-L-L-D; D-L-D-D-L; D-L-L-D-L; or L-D-D-L-D.

In some embodiments, an arginine is adjacent to an amino acid having a hydrophobic side chain. In some embodiments, the arginine has the same chirality as the amino acid having a hydrophobic side chain. In some embodiments, at least two arginines are adjacent to each other. In still other embodiments, three arginines are adjacent to each other. In some embodiments, at least two amino acids each having a hydrophobic side chain are adjacent to each other. In other embodiments, at least three amino acids each having a hydrophobic side chain are adjacent to each other. In other embodiments, the CPPs described herein comprise at least two consecutive amino acids each having a hydrophobic side chain and at least two consecutive arginines. In further embodiments, one hydrophobic amino acid is adjacent to one of the arginines. In still other embodiments, the CPPs described herein comprise at least three consecutive amino acids each having a hydrophobic side chain and there consecutive arginines. In further embodiments, one hydrophobic amino acid is adjacent to one of the arginines. These various combinations of amino acids can have any arrangement of D and L amino acids, e.g., the sequences described above. As used herein, adjacent refers to amino acids that are coupled to each other through a peptide bond.

In some embodiments, any four adjacent amino acids in the CPPs described herein can have one of the following sequences: AA_{H2}-AA_{H1}-R-r, AA_{H2}-AA_{H1}-r-R, R-r-AA_{H1}-AA_{H2}, or r-R-AA_{H1}-AA_{H2}, wherein each of AA_{H1} and AA_{H2} are independently an amino acid having a hydrophobic side chain. Accordingly, in some embodiments, the CPPs used in the polypeptide conjugates described herein have a structure according any of Formula 2A-2D:

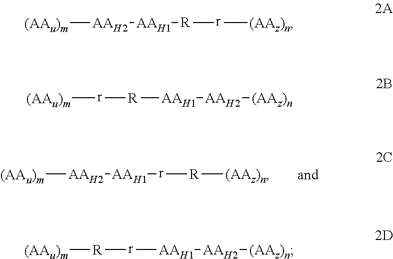

wherein:
each of AA_{H1} and AA_{H2} are independently an amino acid having a hydrophobic side chain;
at each instance and when present, each of AA_{U} and AA_{Z} are independently any amino acid; and
m and n are independently selected from a number from 0 to 6.

In some embodiments, the total number of amino acids (including r, R, AA_{H1}, AA_{H2}), in the CPPs of Formula 2A to 2D are in the range of 6 to 10. In some embodiments, the total number of amino acids is 6. In some embodiments, the total number of amino acids is 7. In some embodiments, the total number of amino acids is 8. In some embodiments, the total number of amino acids is 9. In some embodiments, the total number of amino acids is 10.

In some embodiments of Formula 2A-2D, the sum of m and n is from 2 to 6. In some embodiments, the sum of m and n is 2. In some embodiments, the sum of m and n is 3. In some embodiments, the sum of m and n is 4. In some embodiments, the sum of m and n is 5. In some embodiments, the sum of m and n is 6. In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, n is 0. In some embodiments, n is 1. In some embodiments, n is 2. In some embodiments, n is 3. In some embodiments, n is 4. In some embodiments, n is 5. In some embodiments, n is 6.

In some embodiments, each amino acid having a hydrophobic side chain is independently selected from glycine, alanine, valine, leucine, isoleucine, methionine, phenylalanine, tryptophan, proline, naphthylalanine, phenylglycine, homophenylalanine, tyrosine, cyclohexylalanine, piperidine-2-carboxylic acid, or norleucine, each of which is optionally substituted with one or more substituents. In particular embodiments, each amino acid having a hydrophobic side chain is independently an amino acid having a hydrophobic side chain. In some embodiments, the aromatic hydrophobic amino acid is naphthylalanine, phenylglycine, homophenylalanine, phenylalanine, tryptophan, or tyrosine, each of which is optionally substituted with one or more substituents. In particular embodiments, the amino acid having a hydrophobic side chain is piperidine-2-carboxylic acid, naphthylalanine, tryptophan, or phenylalanine, each of which is optionally substituted with one or more substituents.

In some embodiments of Formula 2A-2D, each of AA_{H1} and AA_{H2} are independently an amino acid having a hydrophobic side chain with a hydrophobicity value which is greater than that of glycine. In other embodiments, each of AA_{H1} and AA_{H2} are independently an amino acid having a hydrophobic side chain with a hydrophobicity value which is greater than that of alanine. In still other embodiments, each of AA_{H1} and AA_{H2} are independently an amino acid having a hydrophobic side chain with a hydrophobicity value which is greater than that of phenylalanine, e.g., as measured using the hydrophobicity scales described above, including Eisenberg and Weiss (Proc. Natl. Acad. Sci. U.S.A. 1984; 81(1):140-144), Engleman, et al. (Ann. Rev. of Biophys. Biophys. Chem. 1986; 1986(15):321-53), Kyte and Doolittle (J. Mol. Biol. 1982; 157(1):105-132), Hoop and Woods (Proc. Natl. Acad. Sci. U.S.A. 1981; 78(6):3824-3828), and Janin (Nature. 1979; 277(5696):491-492), (see Table 1 above). In particular embodiments, hydrophobicity is measured using the hydrophobicity scale reported in Engleman, et al.

The presence of an amino acid having a hydrophobic side chain on the N- or C-terminal of a D-Arg or L-Arg, or a combination thereof, has also found to improve the cytosolic uptake of the CPP (and the attached cargo). For example, in some embodiments, the CPPs (including cCPPs) disclosed herein may include AA_{H1}-D-Arg or D-Arg-AA_{H1}. In other embodiments, the CPPs disclosed herein may include AA_{H1}-L-Arg or L-Arg-AA_{H1}.

The size of the hydrophobic chain of the amino acid on the N- or C-terminal of the D-Arg or an L-Arg, or a combination thereof (i.e., AA_{H1}), may be selected to improve cytosolic delivery efficiency of the CPP. For example, a larger hydrophobic chain on an amino acid on the N- or C-terminal of a D-Arg or L-Arg, or a combination thereof, improves cytosolic delivery efficiency compared to an otherwise identical sequence having a smaller hydrophobic amino acid. The size of the hydrophobic amino acid can be measured in terms of molecular weight of the hydrophobic amino acid, the steric effects of the hydrophobic amino acid, the solvent-accessible surface area (SASA) of the side chain, or combinations thereof. In some embodiments, the size of the hydrophobic amino acid is measured in terms of the molecular weight of the hydrophobic amino acid, and the larger hydrophobic amino acid has a side chain with a molecular weight of at least about 90 g/mol, or at least about 130 g/mol, or at least about 141 g/mol. In other embodiments, the size of the amino acid is measured in terms of the SASA of the hydrophobic side chain, and the larger hydrophobic amino acid has a side chain with a SASA greater than alanine, or greater than glycine.

In other embodiments, AA_{H1} has a hydrophobic side chain with a SASA greater than or equal to about piperidine-2-carboxylic acid, greater than or equal to about tryptophan, greater than or equal to about phenylalanine, or equal to or greater than about naphthylalanine. In some embodiments, AA_{H1} has a side chain side with a SASA of at least about 200 Å², at least about 210 Å2, at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². In some embodiments, AAH₂ has a side chain side with a SASA of at least about 200 Å², at least about 210 Å², at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². In some embodiments, the side chains of AAH₁ and AAH₂ have a combined SASA of at least about 350 Å², at least about 360 Å², at least about 370 Å², at least about 380 Å², at least about 390 Å², at least about 400 Å², at least about 410 Å², at least about 420 Å², at least about 430 Å², at least about 440 Å², at least about 450 Å², at least about 460 Å², at least about 470 Å², at least about 480 Å², at least about 490 Å², greater than about 500 Å², at least about 510 Å², at least about 520 Å², at least about 530 Å², at least about 540 Å², at least about 550 Å², at least about 560 Å², at least about 570 Å², at least about 580 Å², at least about 590 Å², at least about 600 Å², at least about 610 Å², at least about 620 Å², at least about 630 Å², at least about 640 Å², greater than about 650 Å², at least about 660 Å², at least about 670 Å², at least about 680 Å², at least about 690 Å², or at least about 700 Å². In some embodiments, AA_{H2} is a hydrophobic amino acid with a side chain having a SASA that is less than or equal to the SASA of the hydrophobic side chain of AA_{H1}.

By way of example, and not by limitation, a cCPP having a Nal-Arg motif exhibits improved cytosolic delivery efficiency compared to an otherwise identical CPP having a Phe-Arg motif; a cCPP having a Phe-Nal-Arg motif exhibits improved cytosolic delivery efficiency compared to an otherwise identical cCPP having a Nal-Phe-Arg motif; and a phe-Nal-Arg motif exhibits improved cytosolic delivery efficiency compared to an otherwise identical cCPP having a nal-Phe-Arg motif.

As used herein, “hydrophobic surface area” or “SASA” refers to the surface area (reported as square Ångstroms; Å²) of an amino acid side chain that is accessible to a solvent. In particular embodiments, SASA is calculated using the ‘rolling ball’ algorithm developed by Shrake & Rupley (J Mol Biol. 79 (2): 351-71), which is herein incorporated by reference in its entirety for all purposes. This algorithm uses a “sphere” of solvent of a particular radius to probe the surface of the molecule. A typical value of the sphere is 1.4 Å, which approximates to the radius of a water molecule.

SASA values for certain side chains are shown below in Table 3. In certain embodiments, the SASA values described herein are based on the theoretical values listed in Table 3 below, as reported by Tien, et al. (PLOS ONE 8(11): e80635. https://doi.org/10.1371/journal.pone.0080635, which is herein incorporated by reference in its entirety for all purposes.

TABLE 3
SASA Values of Amino Acid Side Chains

| | | | Miller et al. | Rose et al. |
|---|---|---|---|---|
| Residue | Theoretical | Empirical | (1987) | (1985) |
| Alanine | 129.0 | 121.0 | 113.0 | 118.1 |
| Arginine | 274.0 | 265.0 | 241.0 | 256.0 |
| Asparagine | 195.0 | 187.0 | 158.0 | 165.5 |
| Aspartate | 193.0 | 187.0 | 151.0 | 158.7 |
| Cysteine | 167.0 | 148.0 | 140.0 | 146.1 |
| Glutamate | 223.0 | 214.0 | 183.0 | 186.2 |
| Glutamine | 225.0 | 214.0 | 189.0 | 193.2 |
| Glycine | 104.0 | 97.0 | 85.0 | 88.1 |
| Histidine | 224.0 | 216.0 | 194.0 | 202.5 |
| Isoleucine | 197.0 | 195.0 | 182.0 | 181.0 |
| Leucine | 201.0 | 191.0 | 180.0 | 193.1 |
| Lysine | 236.0 | 230.0 | 211.0 | 225.8 |
| Methionine | 224.0 | 203.0 | 204.0 | 203.4 |
| Phenylalanine | 240.0 | 228.0 | 218.0 | 222.8 |
| Proline | 159.0 | 154.0 | 143.0 | 146.8 |
| Serine | 155.0 | 143.0 | 122.0 | 129.8 |
| Threonine | 172.0 | 163.0 | 146.0 | 152.5 |
| Tryptophan | 285.0 | 264.0 | 259.0 | 266.3 |
| Tyrosine | 263.0 | 255.0 | 229.0 | 236.8 |
| Valine | 174.0 | 165.0 | 160.0 | 164.5 |

In some embodiments, the CPP does not include an amino acid with a hydrophobic side chain on the N- and/or C-terminal of AA_{H2}-AA_{H1}-R-r, AA_{H2}-AA_{H1}-r-R, R-r-AA_{H1}-AA_{H2}, or r-R-AA_{H1}-AA_{H2}. In alternative embodiments, the CPP does not include an amino acid having a hydrophobic side chain which is larger (as described herein) than at least one of AA_{H1} or AA_{H2}. In further embodiments, the CPP does not include an amino acid with a hydrophobic side chain having a surface area greater than AA_{H1}. For example, in embodiments in which at least one of AA_{H1} or AA_{H2} is phenylalanine, the cPP does not further include a naphthylalanine (although the CPP include at least one amino acid with a hydrophobic side chain that is smaller than AA_{H1} and AA_{H2}, e.g., leucine). In still other embodiments, the CPP does not include a naphthylalanine in addition to the hydrophobic amino acids in AA_{H2}-AA_{H1}-R-r, AA_{H2}-AA_{H1}-r-R, R-r-AA_{H1}-AA_{H2}, or r-R-AA_{H1}-AA_{H2}.

The chirality of the amino acids (i.e., D or L amino acids) can be selected to improve cytosolic delivery efficiency of the CPP (and the attached cargo as described below). In some embodiments, the hydrophobic amino acid on the N- or C-terminal of an arginine (e.g., AA_{H1}) has the same or opposite chirality as the adjacent arginine. In some embodiments, AA_{H1} has the opposite chirality as the adjacent arginine. For example, when the arginine is D-Arg (i.e. “r”), AA_{H1} is a D-AA_{H1}, and when the arginine is L-Arg (i.e., “R”), AA_{H1} is a L-AA_{H1}. Accordingly, in some embodiments, the CPPs disclosed herein may include at least one of the following motifs: D-AA_{H1}-D-arg, D-arg-D-AA_{H1}, L-AA_{H1}-L-Arg, or L-Arg-LAA_{H1}. In particular embodiments, when arginine is D-arg, AAH can be D-nal, D-trp, or D-phe. In another non-limiting example, when arginine is L-Arg, AAH can be L-Nal, L-Trp, or L-Phe.

In some embodiments, the CPPs (including cCPPs) described herein include three arginines. Accordingly, in some embodiments, the CPPs described herein include one of the following sequences: AA_{H2}-AA_{H1}-R-r-R, AA_{H2}-AA_{H1}-R-r-r, AA_{H2}-AA_{H1}-r-R—R, AA_{H2}-AA_{H1}-r-R-r, R—R-r-AA_{H1}-AA_{H2}, r-R-r-AA_{H1}-AA_{H2}, r-r-R-AA_{H1}-AA_{H2}, or, R-r-R-AA_{H1}-AA_{H2}. In particular embodiments, the CPPS have one of the following sequences AA_{H2}-AA_{H1}-R-r-R, AA_{H2}-AA_{H1}-r-R-r, r-R-r-AA_{H1}-AA_{H2}, or R-r-R-AA_{H1}-AA_{H2}. In some embodiments, the chirality of AAH₁ and AAH₂ can be selected to improve cytosolic uptake efficiency, e.g., as described above, where AAH₁ has the same chirality as the adjacent arginine, and AAH₁ and AAH₂ have the opposite chirality.

In some embodiments, the CPPs described herein include at least three amino acids having a hydrophobic side chain. Accordingly, in some embodiments, the CPPs described herein include one of the following sequences: AA_{H3}-AA_{H2}-AA_{H1}-R-r, AA_{H3}-AA_{H2}-AA_{H1}-R-r, AA_{H3}-AA_{H2}-AA_{H1}-r-R, AA_{H3}-AA_{H2}-AA_{H1}-r-R, R-r-AA_{H1}-AA_{H2}-AA_{H3}, R-r-AA_{H1}-AA_{H2}-AA_{H3}, r-R-AA_{H1}-AA_{H2}-AA_{H3}, or, r-R-AA_{H1}-AA_{H2}-AA_{H3}, wherein AA_{H3} is any amino acid having a hydrophobic side chain described above, e.g., piperidine-2-carboxylic acid, naphthylalanine, tryptophan, or phenylalanine. In some embodiments, the chirality of AA_{H1}, AA_{H2}, and AA_{H3} can be selected to improve cytosolic uptake efficiency, e.g., as described above, where AA_{H1} has the same chirality as the adjacent arginine, and AA_{H1} and AA_{H2} have the opposite chirality. In other embodiments, the size of AA_{H1}, AA_{H2}, and AA_{H3} can be selected to improve cytosolic uptake efficiency, e.g., as described above, where AA_{H3} has a SASA of less than or equal to AA_{H1} and/or AA_{H2}.

In some embodiments, AA_{H1} and AA_{H2} have the same or opposite chirality. In certain embodiments, AA_{H1} and AA_{H2} have the opposite chirality. Accordingly, in some embodiments, the CPPs disclosed herein include at least one of the following sequences: D-AA_{H2}-L-AA_{H1}-R-r; L-AA_{H2}-D-AA_{H1}-r-R; R-r-D-AA_{H1}-L-AA_{H2}; or r-R-L-AA_{H1}-D-AA_{H1}, wherein each of D-AA_{H1} and D-AA_{H2} is a hydrophobic amino acid having a D configuration, and each of L-AA_{H1} and L-AA_{H2} is a hydrophobic amino acid having an L configuration. In some embodiments, each of D-AA_{H1} and D-AA_{H2} is independently selected from the group consisting of D-pip, D-nal, D-trp, and D-phe. In particular embodiments, D-AA_{H1} or D-AA_{H2} is D-nal. In other particular embodiments, D-AA_{H1} is D-nal. In some embodiments, each of L-AA_{H1} and L-AA_{H2} is independently selected from the group consisting of L-Pip, L-Nal, L-Trp, and L-Phe. In particular embodiments, each of L-AA_{H1} and L-AA_{H2} is L-Nal. In other particular embodiments, L-AA_{H1} is L-Nal.

As discussed above, the disclosure provides for various modifications to a cyclic peptide sequence which may improve cytosolic delivery efficiency. In some embodiments, improved cytosolic uptake efficiency can be measured by comparing the cytosolic delivery efficiency of the polypeptide conjugate or complex of the present disclosure having the modified sequence to a proper control sequence. In some embodiments, the control sequence does not include a particular modification (e.g., matching chirality of R and AA_{H1}) but is otherwise identical to the modified sequence. In other embodiments, the control has the following sequence: cyclic (FΦRRRRQ)

As used herein cytosolic delivery efficiency refers to the ability of a polypeptide conjugate or complex of the present disclosure to traverse a cell membrane and enter the cytosol. In embodiments, cytosolic delivery efficiency of the CPP is not dependent on a receptor or a cell type. Cytosolic delivery efficiency can refer to absolute cytosolic delivery efficiency or relative cytosolic delivery efficiency.

Absolute cytosolic delivery efficiency is the ratio of cytosolic concentration of a polypeptide conjugate or complex of the present disclosure over the concentration of the polypeptide conjugate or complex of the present disclosure in the growth medium. Relative cytosolic delivery efficiency refers to the concentration of a polypeptide conjugate or complex of the present disclosure in the cytosol compared to the concentration of a control polypeptide conjugate or complex of the present disclosure in the cytosol. Quantification can be achieved by fluorescently labeling the polypeptide conjugate or complex of the present disclosure (e.g., with a FTIC dye) and measuring the fluorescence intensity using techniques well-known in the art.

In particular embodiments, relative cytosolic delivery efficiency is determined by comparing (i) the amount of a polypeptide conjugate or complex of the present disclosure internalized by a cell type (e.g., HeLa cells) to (ii) the amount of the polypeptide conjugate or complex of the present disclosure internalized by the same cell type. To measure relative cytosolic delivery efficiency, the cell type may be incubated in the presence of a cell-penetrating peptide of the invention for a specified period of time (e.g., 30 minutes, 1 hour, 2 hours, etc.) after which the amount of the polypeptide conjugate or complex of the present disclosure internalized by the cell is quantified using methods known in the art, e.g., fluorescence microscopy. Separately, the same concentration of the control is incubated in the presence of the cell type over the same period of time, and the amount of the control internalized by the cell is quantified.

Non-limiting examples of suitable cyclic cell penetrating peptide are provided in Table 4.

TABLE 4
Examples of Cyclic Cell-Penetrating Peptides (cCPPs)

| | | SEQ |
|---|---|---|
| | | ID |
| ID | cCPP Sequence | NO |
| PCT 1 | cyclo(FΦRRRQ) | 1 |
| PCT 2 | cyclo(FΦRRRC) | 2 |
| PCT 3 | cyclo(FΦRRRU) | 3 |
| PCT 4 | cyclo(RRRΦFQ) | 4 |
| PCT 5 | cyclo(RRRRΦF) | 5 |
| PCT 6 | cyclo(FΦRRRR) | 6 |
| PCT 7 | cyclo(FφrRrRq) | 7 |
| PCT 8 | cyclo(FφrRrRQ) | 8 |
| PCT 9 | cyclo(FΦRRRRQ) | 9 |
| PCT 10 | cyclo(fΦRrRrQ) | 10 |
| PCT 11 | cyclo(RRFRΦRQ) | 11 |
| PCT 12 | cyclo(FRRRRΦQ) | 12 |
| PCT 13 | cyclo(rRFRΦRQ) | 13 |
| PCT 14 | cyclo(RRΦFRRQ) | 14 |
| PCT 15 | cyclo(CRRRRFWQ) | 15 |
| PCT 16 | cyclo(FfΦRrRrQ) | 16 |
| PCT 17 | cyclo(FFΦRRRRQ) | 17 |
| PCT 18 | cyclo(RFRFRΦRQ) | 18 |
| PCT 19 | cyclo(URRRRFWQ) | 19 |
| PCT 20 | cyclo(CRRRRFWQ) | 20 |
| PCT 21 | cyclo(FΦRRRRQK) | 21 |
| PCT 22 | cyclo(FΦRRRRQC) | 22 |
| PCT 23 | cyclo(fΦRrRrRQ) | 23 |
| PCT 24 | cyclo(FΦRRRRRQ) | 24 |
| PCT 25 | cyclo(RRRRΦFDΩC) | 25 |
| PCT 26 | cyclo(FΦRRR) | 26 |
| PCT 27 | cyclo(FWRRR) | 27 |
| PCT 28 | cyclo(RRRΦF) | 28 |
| PCT 29 | cyclo(RRRWF) | 29 |
| SAR 1 | cyclo(FΦRRRRQ) | 30 |
| SAR 19 | cyclo(FFRRRQ) | 31 |
| SAR 20 | cyclo(FFrRrQ) | 32 |
| SAR 21 | cyclo(FFRrRQ) | 33 |
| SAR 22 | cyclo(FRFRRQ) | 34 |
| SAR 23 | cyclo(FRRFRQ) | 35 |
| SAR 24 | cyclo(FRRRFQ) | 36 |
| SAR 25 | cyclo(GΦRRRQ) | 37 |
| SAR 26 | cyclo(FFFRAQ) | 38 |
| SAR 27 | cyclo(FFFRRQ) | 39 |
| SAR 28 | cyclo(FFRRRRQ) | 40 |
| SAR 29 | cyclo(FRRFRRQ) | 41 |
| SAR 30 | cyclo(FRRRFRQ) | 42 |
| SAR 31 | cyclo(RFFRRRQ) | 43 |
| SAR 32 | cyclo(RFRRFRQ) | 44 |
| SAR 33 | cyclo(FRFRRRQ) | 45 |
| SAR 34 | cyclo(FFFRRRQ) | 46 |
| SAR 35 | cyclo(FFRRRFQ) | 47 |
| SAR 36 | cyclo(FRFFRRQ) | 48 |
| SAR 37 | cyclo(RRFFFRQ) | 49 |
| SAR 38 | cyclo(FFRFRRQ) | 50 |
| SAR 39 | cyclo(FFRRFRQ) | 51 |
| SAR 40 | cyclo(FRRFFRQ) | 52 |
| SAR 41 | cyclo(FRRFRFQ) | 53 |
| SAR 42 | cyclo(FRFRFRQ) | 54 |
| SAR 43 | cyclo(RFFRFRQ) | 55 |
| SAR 44 | cyclo(GΦRRRRQ) | 56 |
| SAR 45 | cyclo(FFFRRRRQ) | 57 |
| SAR 46 | cyclo(RFFRRRRQ) | 58 |
| SAR 47 | cyclo(RRFFRRRQ) | 59 |
| SAR 48 | cyclo(RFFFRRRQ) | 60 |
| SAR 49 | cyclo(RRFFFRRQ) | 61 |
| SAR 50 | cyclo(FFRRFRRQ) | 62 |
| SAR 51 | cyclo(FFRRRRFQ) | 63 |
| SAR 52 | cyclo(FRRFFRRQ) | 64 |
| SAR 53 | cyclo(FFFRRRRRQ) | 65 |
| SAR 54 | cyclo(FFFRRRRRRQ) | 66 |
| SAR 55 | cyclo(FΦRrRrQ) | 67 |
| SAR 56 | cyclo(XXRRRRQ) | 68 |
| SAR 57 | cyclo(FfFRrRQ) | 69 |
| SAR 58 | cyclo(fFfrRrQ) | 70 |
| SAR 59 | cyclo(fFfRrRQ) | 71 |
| SAR 60 | cyclo(FfFrRrQ) | 72 |
| SAR 61 | cyclo(fFφrRrQ) | 73 |
| SAR 62 | cyclo(fΦfrRrQ) | 74 |
| SAR 63 | cyclo(φFfrRrQ) | 75 |
| SAR 64 | cyclo(FΦrRrQ) | 76 |
| SAR 65 | cyclo(fΦrRrQ) | 77 |
| SAR 66 | Ac-(Lys-fFRrRrD) | 78 |
| SAR 67 | Ac-(Dap-fFRrRrD) | 79 |
| SAR 68 | 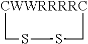 | 80 |
| SAR 69 | 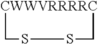 | 81 |
| SAR 70 | 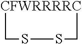 | 82 |
| SAR 71 | 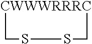 | 83 |
| Pin1 15 | cyclo(Pip-Nal-Arg-Glu-arg-arg-glu) | 84 |
| Pin1 16 | cyclo(Pip-Nal-Arg-Arg-arg-arg-glu) | 85 |
| Pin1 17 | cyclo(Pip-Nal-Nal-Arg-arg-arg-glu) | 86 |
| Pin1 18 | cyclo(Pip-Nal-Nal-Arg-arg-arg-Glu) | 87 |
| Pin1 19 | cyclo(Pip-Nal-Phe-Arg-arg-arg-glu) | 88 |
| Pin1 20 | cyclo(Pip-Nal-Phe-Arg-arg-arg-Glu) | 89 |
| Pin1 21 | cyclo(Pip-Nal-phe-Arg-arg-arg-glu) | 90 |
| Pin1 22 | cyclo(Pip-Nal-phe-Arg-arg-arg-Glu) | 91 |
| Pin1 23 | cyclo(Pip-Nal-nal-Arg-arg-arg-Glu) | 92 |
| Pin1 24 | cyclo(Pip-Nal-nal-Arg-arg-arg-glu) | 93 |
| Rev-13 | [Pim-RQRR-Nlys]GRRR^{b} | 94 |
| hLF | 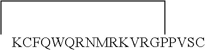 | 95 |
| cTat | [KrRrGrKkRrE]^{c} | 96 |
| cR10 | [KrRrRrRrRrRE]^{c} | 97 |
| L-50 | [RVRTRGKRRIRRpP] | 98 |
| L-51 | [RTRTRGKRRIRVpP] | 99 |
| [WR]₄ | [WRWRWRWR] | 100 |
| MCOTI- II | 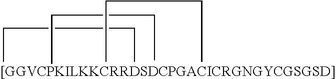 | 101 |
| Rotstein | [P-Cha-r-Cha-r-Cha-r-Cha-r-G]^{d} | 102 |
| et al. | | |
| Chem. | | |
| Eur. J. | | |
| 2011 | | |
| Lian et | Tm(SvP-F₂Pmp-H)-Dap-(FΦRRRR-Dap)]^{f} | 103 |
| al. | | |
| J. Am. | | |
| Chem. | | |
| Soc. | | |
| 2014 | | |
| Lian et | [Tm(a-Sar-D-pThr-Pip-ΦRAa)-Dap-(FΦRRRR-Dap)]^{f} | 104 |
| al. | | |
| J. Am. | | |
| Chem. | | |
| Soc. | | |
| 2014 | | |
| IA8b | [CRRSRRGCGRRSRRCG]^{g} | 105 |
| Dod- | [K(Dod)RRRR] | 106 |
| [R₅] | | |
| LK-3 | LKKLCKLLKKLCKLAG | 107 |
| | L\|KKLCKLL\|KKLCKLAG | |
| | RRRR-[KRRRE]^{c} | 108 |
| | RRR-[KRRRRE]^{c} | 109 |
| | RR-[KRRRRRE]^{c} | 110 |
| | R-[KRRRRRRE]^{c} | 111 |
| [CR]₄ | [CRCRCRCR] | 112 |
| cyc3 | [Pra-LRKRLRKFRN-AzK]^{h} | 113 |
| PMB | T-Dap-[Dap-Dap-f-L-Dap-Dap-T] | 114 |
| GPMB | T-Agp-[Dap-Agp-f-L-Agp-Agp-T] | 115 |
| cCPP1 | cyclo(FΦRRRRQ) | 116 |
| cCPP12 | cyclo(FfΦRrRrQ) | 117 |
| cCPP9 | cyclo(fΦRrRrQ) | 118 |
| cCPP11 | cyclo(fΦRrRrRQ) | 119 |
| cCPP18 | cyclo(FφrRrRq) | 120 |
| cCPP13 | cyclo(FφrRrRQ) | 121 |
| cCPP6 | cyclo(FΦRRRRRQ) | 122 |
| cCPP3 | cyclo(RRFRΦRQ) | 123 |
| cCPP7 | cyclo(FFΦRRRRQ) | 124 |
| cCPP8 | cyclo(RFRFRΦRQ) | 125 |
| cCPP5 | cyclo(FΦRRRQ) | 126 |
| cCPP4 | cyclo(FRRRRΦQ) | 127 |
| cCPP10 | cyclo(rRFRΦRQ) | 128 |
| cCPP2 | cyclo(RRΦFRRQ) | 129 |

Φ, L-2-naphthylalanine; φ, D-2-naphthylalanine; Pim, pimelic acid; Nlys, lysine peptoid residue; D-pThr, D-phosphothreonine; Pip, L-piperidine-2-carboxylic acid; Cha, L-3-cyclohexyl-alanine; Tm, trimesic acid; Dap, L-2,3-diaminopropionic acid; Sar, sarcosine; F₂Pmp, L-difluorophosphonomethyl phenylalanine; Dod, dodecanoyl; Pra, L-propargylglycine; AzK, L-6-Azido-2-amino-hexanoic; Agp, L-2-amino-3-guanidinylpropionic acid; ^{b}Cyclization between Pim and Nlys; ^{c}Cyclization between Lys and Glu; ^{d}Macrocyclization by multicomponent reaction with aziridine aldehyde and isocyanide; ^{e}Cyclization between the main-chain of Gln residue; ^{f}N-terminal amine and side chains of two Dap residues bicyclized with Tm; ^{g}Three Cys side chains bicyclized with tris(bromomethyl)benzene; ^{h}Cyclization by the click reaction between Pra and Azk.

Additionally, the cCPP used in the polypeptide conjugates and methods described herein can include any sequence disclosed in: U.S. application Ser. No. 15/312,878; U.S. application Ser. No. 15/360,719; U.S. App. No. 62/438,141, and U.S. App. No. 62/507,483, each of which is incorporated by reference in its entirety for all purposes.

Group that Binds to the Cargo by Electrostatic Interactions (P)

The polypeptide conjugate of the present disclosure comprises
a) a group that binds to a nucleic acid sequence (“cargo”) by electrostatic interactions (P) comprising at least one peptide or polyamine; and
b) at least one cell-penetrating peptide (CPP);
wherein each peptide comprises at least three monomers selected from arginine, arginine-analog, lysine, lysine-analog, histidine, or histidine-analog; wherein the P is conjugated to the CPP through a bond or at least one linker (L); and
wherein the polypeptide conjugate is optionally charged.

In one embodiment, the group that electrostatically interacts with the cargo (P) comprises a polyarginine peptide (pArg). In some embodiments, the polyarginine peptide comprises three arginine monomers or arginine-analog monomers. In some embodiments, the polyarginine peptide comprises four arginine monomers or arginine-analog monomers. In some embodiments, the polyarginine peptide comprises five arginine monomers or arginine-analog monomers. In some embodiments, the polyarginine peptide comprises 3 to 50 arginine monomers or arginine-analog monomers.

In some embodiments, P comprises polyarginine peptide comprising 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 ariginine monomers or arginine-analog monomers.

In some embodiments, P comprises a polyarginine peptide having the following repeating units where n is an integer selected from 1 to 100 and the polyarginine peptide can have one additional arginine monomer to make an odd-numbered chain.

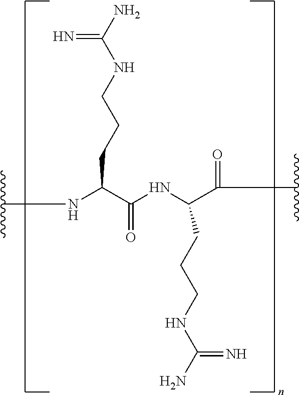

In some embodiments, P comprises a polyarginine peptide having the following repeating units where n is an integer selected from 1 to 50. In some embodiment, n is 1, 2, 3, 4, or 5.

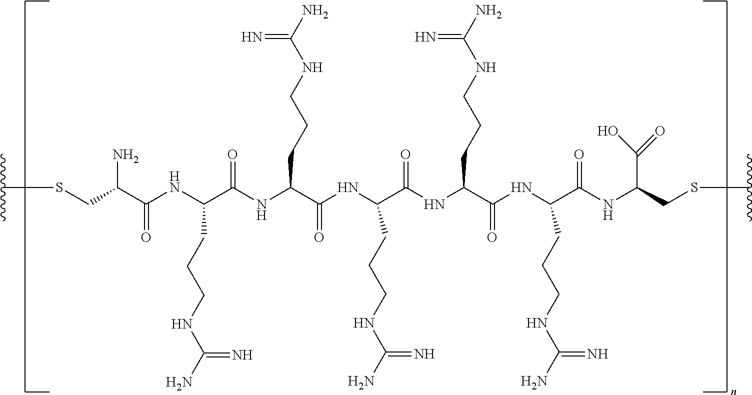

In one embodiment, the group that electrostatically interacts with the cargo (P) comprises a polylysine peptide (pLys). In some embodiments, the polylysine peptide comprises three lysine monomers or lysine-analog monomers. In some embodiments, the polylysine peptide comprises four lysine monomers or lysine-analog monomers. In some embodiments, the polylysine peptide comprises five lysine monomers or lysine-analog monomers. In some embodiments, the polylysine peptide comprises 3 to 50 lysine monomers or lysine-analog monomers.

In some embodiments, P comprises polylysine peptide comprising 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 lysine monomers or lysine-analog monomers.

In one embodiment, the group that electrostatically interacts with the cargo (P) comprises a polyhistidine peptide (pHis). In some embodiments, the polyhistidine peptide comprises three histidine monomers or histidine-analog monomers. In some embodiments, the polyhistidine peptide comprises four histidine monomers or histidine-analog monomers. In some embodiments, the polyhistidine peptide comprises five histidine monomers or histidine-analog monomers. In some embodiments, the polyhistidine peptide comprises 3 to 50 histidine monomers or histidine-analog monomers.

In some embodiments, P comprises polyhistidine peptide comprising 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 histidine monomers or histidine-analog monomers.

In one embodiment, the group that electrostatically interacts with the cargo (P) comprises a peptide comprises monomers selected from arginine, arginine-analog, lysine, lysine-analog, histidine, histidine-analog, or mixtures thereof. In some embodiments, the peptide comprises three monomers selected from arginine, arginine-analog, lysine, lysine-analog, histidine, or histidine-analog. In some embodiments, the peptide comprises four monomers selected from arginine, arginine-analog, lysine, lysine-analog, histidine, or histidine-analog. In some embodiments, the peptide comprises five monomers selected from arginine, arginine-analog, lysine, lysine-analog, histidine, or histidine-analog. In some embodiments, the peptide comprises 3 to 50 monomers selected from arginine, arginine-analog, lysine, lysine-analog, histidine, or histidine-analog.

In some embodiments, P comprises a peptide comprising 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 monomers selected from arginine, arginine-analog, lysine, lysine-analog, histidine, or histidine-analog. In some embodiments, arginine, arginine-analog, lysine, lysine-analog, histidine, or histidine-analog monomers can be in any order, D or L, or permutation thereof. In some embodiment, the monomers alternate D and L in the peptide sequence.

In some embodiments, P comprises a polyamine selected from a spermidine polymer or a spermine polymer. In some embodiments, P comprises a polyamine structure:

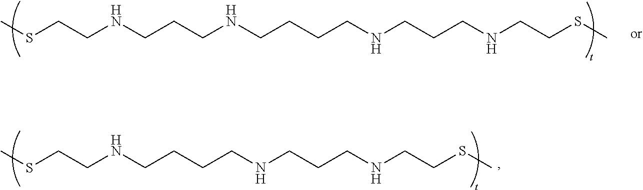

wherein t is an integer selected from 1-50, or a charged species thereof.

In some embodiments, P further comprises at least one cysteine monomer or a cysteine-analog monomer. In some embodiments, P further comprises at least two cysteine monomers or cysteine-analog monomers.

In some embodiments, P further comprises at least one group selected from:

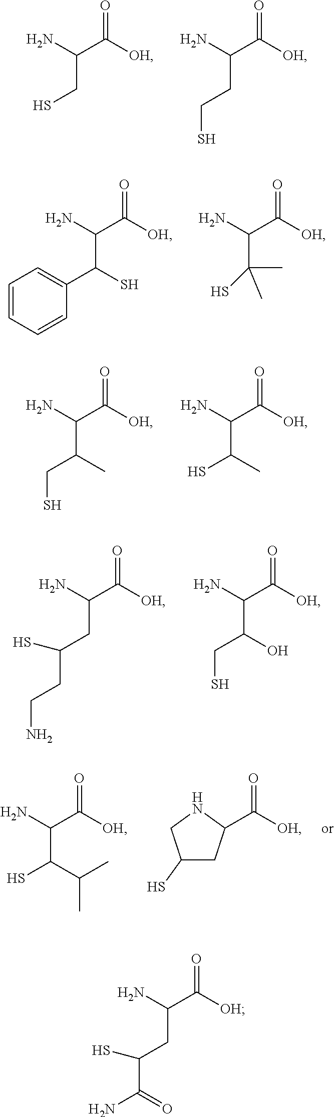

wherein the bond to the hydrogen on one at least one of the N- or C-termini is replaced by a bond to the peptide or polyamine; and
wherein the bond to the hydrogen on the thiol group is replaced by a bond to the CPP.

In some embodiments, P further comprises at least one group selected from:

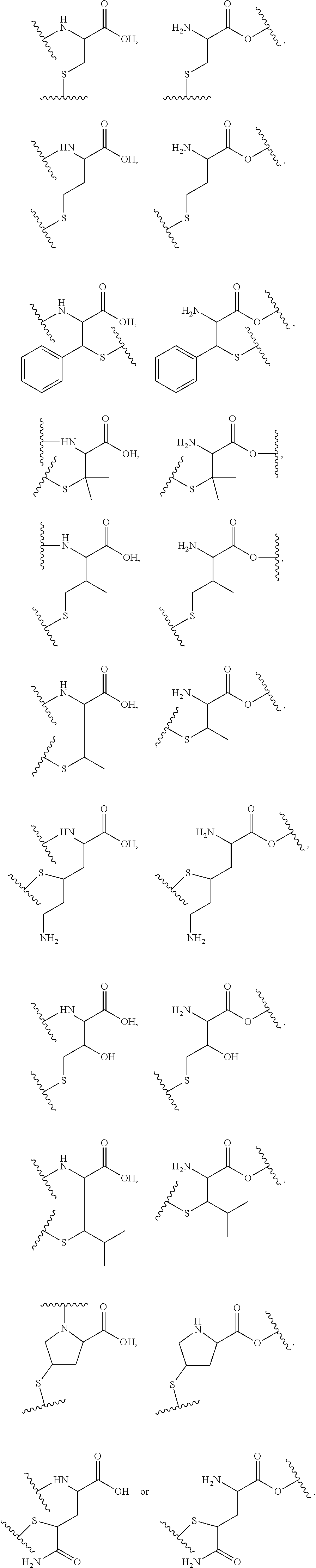

In some embodiments, at least one of the P (including P¹ and P²) in the polypeptide conjugate as disclosed herein further comprises

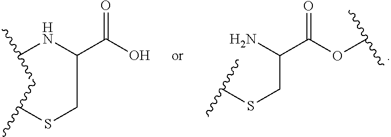 [SMILES: *NC(CS*)C(=O)O ; *OC(=O)C(N)CS*]

In other embodiments, at least one of the P in the polypeptide conjugate as disclosed herein further comprises at least two groups selected form

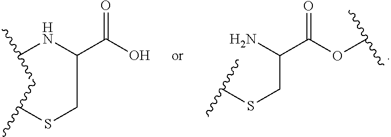 [SMILES: *NC(CS*)C(=O)O ; *OC(=O)C(N)CS*]

In some embodiments, P (including P¹ and P²) further comprises one or more β-alanine monomers. In one embodiment, P (including P¹ and P²) further comprises at least one amino acid monomer (e.g., such as those shown in Table 4). In one embodiment, P (including P¹ and P²) further comprises a thioether moiety (—S—). In some embodiments, P is pArg further comprising one or more β-alanine monomers.

In some embodiments, P (including P¹ and P²) comprises -Cys-(Arg)_{X}-Cys- (SEQ ID NO: 131), -Cys-βAla-(Arg)_{X}-Cys- (SEQ ID NO: 132), -Cys-(Arg)_{X}-βAla-Cys- (SEQ ID NO: 133), -Cys-βAla-(Arg)_{X}-βAla-Cys- (SEQ ID NO: 134), -Cys-(polyamine)_{X}-Cys-, -Cys-βAla-(polyamine)_{X}-Cys-, -Cys-(polyamine)_{X}-βAla-Cys-, -Cys-βAla-(polyamine)_{X}-βAla-Cys- (SEQ ID NO: 135), —S-(polyamine)_{X}-S—, wherein x=3, 4, 5, 6, 7, or 8.

In some embodiments, “-” between different groups of P (including [P]ₙ, [P]ₘ and [P]ₒ) represents a bond between two sulfur atoms (a disulfide bond).

In some embodiments, P, P¹, or P², at each occurrence, are same or different.

In some embodiments, P is charged. In some embodiments, P is positively charged. In some embodiments, P is positively charged and interact with negatively charged nucleic acids by non-covalent interactions. In some embodiments, P is positively charged and interact with negatively charged nucleic acids by electrostatic interactions.

In some embodiments, P binds (by electrostatic interactions) to a cargo to form 1:1 (mol:mol) complex. Without being bound to any theory, the complex formed between P and the cargo (e.g., nucleic acid) can protect the cargo and P from enzymatic degradation during storage or in vivo circulation.

In some embodiments, P is biodegradable. In some embodiments, P can undergo reductive cleavage inside the cytosol. In some embodiments, the reductive cleavage is by cleavage of the disulfide bonds. In some embodiment, the degradation is by proteolytic degradation. In some embodiments, P can release the cargo inside the cytosol.

### Linkers (L)

In some embodiments, the polypeptide conjugate comprises a linker (L) that connects the CPP to the P. In some embodiment of the polypeptide conjugate as disclosed herein, at least one L comprises a divalent optionally substituted group selected from amino acid, alkylene, alkenylene, alkynylene, aryl, cycloalkyl, cycloalkenyl, cycloalkynyl, heterocyclyl, heteroaryl, —(R¹—X—R²)z-, or combinations thereof; wherein
each of R¹ and R² are independently selected from a bond, alkylene, alkenylene, alkynylene, carbocyclyl, and heterocyclyl, wherein R¹ and R² are not both a bond;
each X is independently N, S, and O; and
z is an integer selected from 1 to 20.

In some embodiments of the polypeptide conjugate as disclosed herein, at least one L comprises a divalent polyethylene glycol moiety. In some embodiments, at least one L comprises an optionally substituted —(O—CH₂CH₂)z- or an optionally substituted —(CH₂CH₂—O)z-. In some embodiments, at least one L comprises a divalent 8-amino-3,6-dioxaoctanoic acid residue. In other embodiments, at least one L comprises a divalent 8-amino-3,6,9-trioxaundecanoic acid residue.

In some embodiments, L comprises one or more groups selected from:

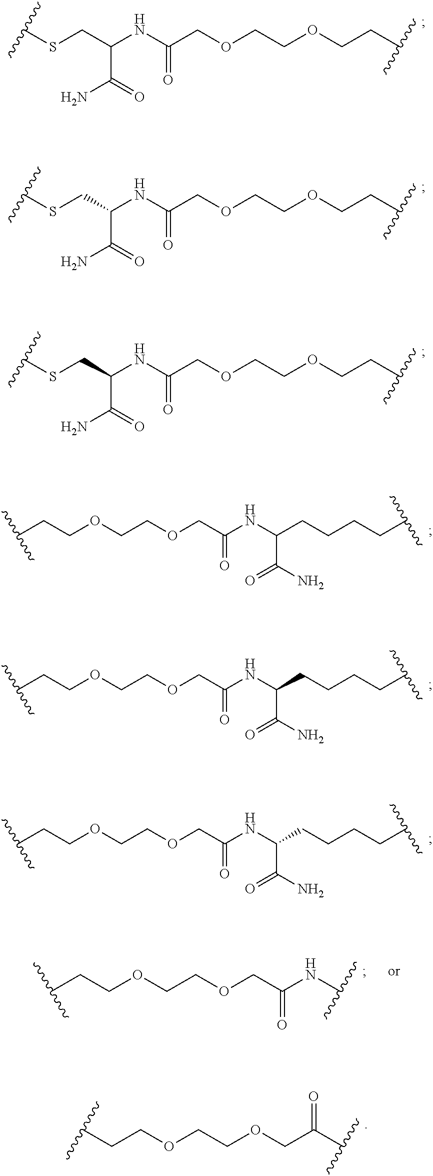

In some embodiments, at least one L comprises a physiological cleavable group (PCG). In some embodiments, each PCG is, independently, selected from —S—S—, carbonate (—O—C(O)O—), thiocarbonate (—O—C(S)O—), thioester (—C(S)O—), sulfoxide (—S(O)—), hydrazine (—H₂N—NH₂—), or protease-cleavable dipeptide linker. In other embodiments, each PCG comprises at least one —S—S—.

In some embodiments, L comprises two or more PCGs. Non-limiting examples of such L include dendrimers (e.g., cyclotriphosphazene, polypropylenimine, polylysine, and polyamidoamine dendrimers having appropriate terminal groups to form a PCG), dendrons (e.g., 2-bis(hydroxymethyl) propanyl-based dendrons having appropriate terminal groups to form a PCG), and hyperbranched polymers (e.g., hyperbranched bis-MPA polyester ving appropriate terminal groups to form a PCG).

In some embodiments of any one of the polypeptide conjugate as disclosed herein, at least one of the “-” between L (including L¹ and L²) and P (including [P]ₙ [P¹]ₚ, [P²]_{q} and ([P]ₘ)ₛ) represents a bond between two sulfur atoms (disulfide bond). In other embodiments, the “-” between L and P each represents a bond between two sulfur atoms (disulfide bond).

In some embodiments, L, L¹, or L², at each occurrence, are same or different.

### Complex

The present disclosure also relates to a complex comprising any one of the polypeptide conjugate as disclosed herein and a nucleic acid sequence (also referred to as cargo).

In some embodiments, the cargo is charged. In some embodiments, the cargo is negatively charged. In some embodiments, the negatively charged cargo interacts with positively charged P by non-covalent interactions. In some embodiments, the negatively charged cargo interacts with positively charged P by electrostatic interactions.

In some embodiments, the cargo is a nucleic acid. In some embodiments, the cargo is a nucleic acid sequence. In some embodiment, the nucleic acid or the nucleic acid sequence is therapeutically active or therapeutically effective.

In some embodiments, the cargo comprises therapeutically active agent for a gene therapy. In some embodiments, the cargo comprises splice-switching oligonucleotides, microRNAs, anti-microRNAs, antisense oligonucleotides, small interfering DNAs, plasmid DNAs, small interfering RNAs and/or mRNAs.

In some embodiments, the cargo comprises therapeutically active agent for a gene-editing.

In some embodiments, the cargo can enhance target gene expression or modulate/or switch mRNA splicing to express desired gene products.

The present disclosure also relates to a cell comprising any one of the polypeptide conjugate as disclosed herein. The present disclosure also relates to a cell comprising a complex comprising any one of the polypeptide conjugate as disclosed herein and a nucleic acid sequence.

### Polypeptide Conjugate as a Delivery Device

The present disclosure also relates to a method of delivering the cargo (e.g., a nucleic acid sequence) to a cell, comprising contacting the cell with any one of the polypeptide conjugate complex as disclosed herein.

The present disclosure also relates to a method of delivering the cargo to a cell of a subject in need thereof, comprising administering any one of the polypeptide conjugate complex as disclosed herein.

The present disclosure also relates to a method of treating a disease or condition in a patient in need thereof, comprising administering any one of the polypeptide conjugate complex as disclosed herein to the patient. The disease or condition may be any disease or condition that can be treated by gene replacement or gene therapy. In some embodiment, the disease or condition is cancer, genetic diseases, autoimmunity, inflammatory diseases, neurodegenerative diseases, or infectious diseases.

### EXAMPLES

### Example 1. Polypeptide Conjugate with Two Terminal cCPPs

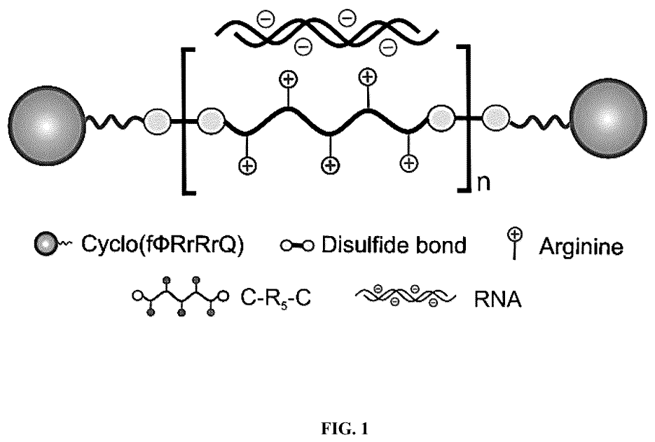

Design and Synthesis. A non-covalent complex between cyclic CPP-based delivery vector and a nucleic acid (NA) cargo of interest instead of covalent attachment was selected for a number of reasons. First, in a covalent CPP-nucleic acid conjugate, the negatively charged nucleic acid may interact with the positively charged CPP (either intramolecularly or intermolecularly) and mutually interfere with each other's function. Second, in a covalent adduct, the nucleic acid cargo is unprotected from nuclease action and may have limited in vivo stability. Third, production of a covalent conjugate is more complex, requiring chemical synthesis of each component followed by biorthogonal conjugation, whereas a non-covalent complex can be formed by simply mixing the two components. However, it was also desired to avoid the formation of nanoparticles, which are largely limited to biodistribution into the liver and kidney (see Juliano, R. L. Nucleic Acids Res., 2016, 44(14): 6518-6548; Shi, B. et al., J Histochem Cytochem., 2011, 59(8): 727-740). With these considerations in mind, a novel NA delivery system was designed consisting of a biodegradable polyarginine sequence flanked by two cyclic CPPs, which are copolymerized through the formation of a series of disulfide bonds (FIG. 1). The polyarginine moiety was expected to bind tightly to a NA cargo (e.g., siRNA) through electrostatic interactions to form a 1:1 (mol/mol) complex, while the two terminal CPPs would be exposed for binding to the cell membrane and ensuring cellular entry of the complex. Non-covalent complexation could allow the same vector to deliver different siRNA sequences (and potentially other NAs).

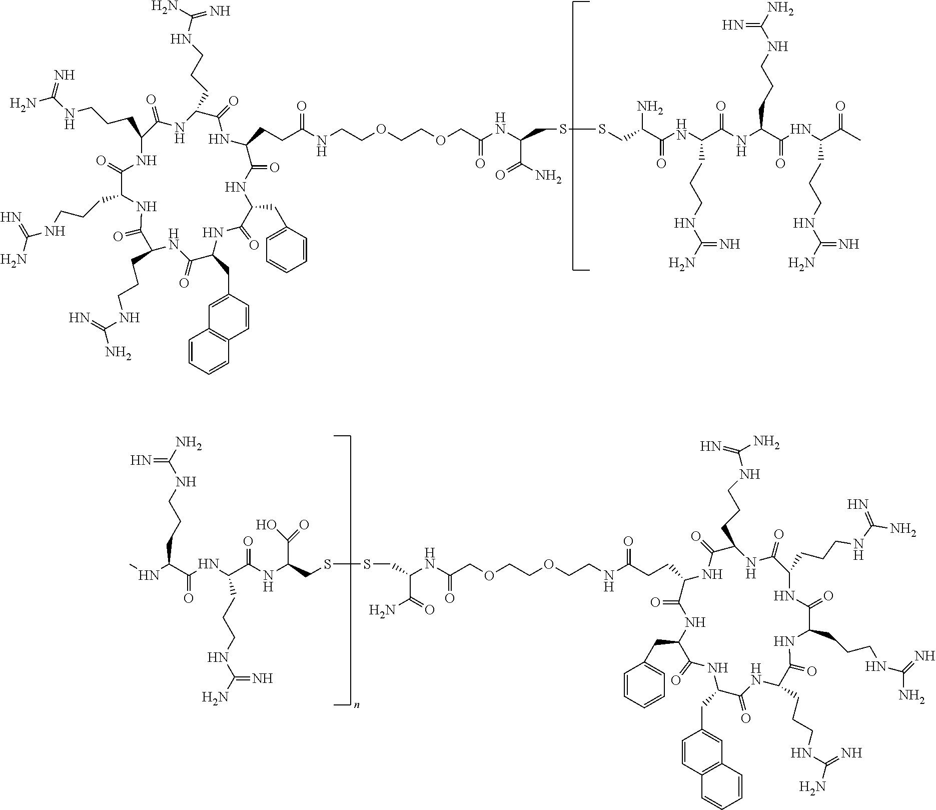

CPP9 (see Table 4), which has a cytosolic delivery efficiency of 62%, was selected for this study. To minimize any mutual interference between the CPP and the cargo, a long, flexible linker (miniPEG) was attached to the Gln side chain and a cysteine was added to the other end of the linker. The polyarginine sequence was formed by polymerization of Cys-Arg-Arg-Arg-Arg-Arg-Cys (SEQ ID NO: 136) (CR₅C) through the formation of disulfide bonds. A pentaarginine was selected because longer polyarginines (e.g., R₁₀), without bound to any theory, would bind more tightly to NAs and may not effectively release the cargo inside the cell.

Peptides CR₅C (SEQ ID NO: 136) and cyclo(fΦRrRrQ)-miniPEG-Cys (SEQ ID NO: 141) were synthesized manually on Rink amide resin (0.54 mmol/g) using standard Fmoc chemistry. The typical coupling reaction contained 5 eq of Fmoc-amino acid, 5 eq HATU and 10 eq DIPEA in DMF with mixing for 1 h. The N-terminal Fmoc group was removed by 20% piperidine in DMF. For cyclo(fΦRrRrQ) peptide (SEQ ID NO: 118), after the entire sequence was completed but prior to Fmoc deprotection, the allyl group on the C-terminal Glu was removed by treatment with 0.1 eq Pd(PPh₃)₄ and 10 eq phenylsilane in DCM (3×15 min). The N-terminal Fmoc was then removed and the peptide was cyclized by treatment with 5 eq PyBOP, 5 eq HOBt and 10 eq DIPEA in DMF for 3 h. The peptides were cleaved from the resin and side chain deprotected by treatment with 90/2.5/2.5/2.5/2.5 (v/v) TFA/TIPS/DMB/DCM/EDT/water for 3 h. The peptides were triturated with cold ethyl ether and purified by reversed-phase HPLC. The purity of peptides (>95%) was confirmed with an analytical reversed-phase HPLC and the identity of peptides was confirmed by MALDI-TOF mass spectrometry.

Three different polymerization reactions were carried out by mixing 5, 10 or 20 equivalents of CR₅C and 1 equivalent of CPP9-miniPEG-Cys in PBS (pH 7.4) containing 30% DMSO and stirring the mixture for 24 h. The reaction products were dialyzed against a semipermeable membrane (MWCO: 10 kDa) to remove DMSO and low-MW species. The resulting polymers were lyophilized. The expected MWs of the polymers are listed in Table 5.

TABLE 5
Theoretical molecular weight (MW) of CRC polymers

| Polymer | Mole ratio of CR₅C per CPP9 | Theoretical MW (kDa) |
|---|---|---|
| CRC5 | 5 | 12.7 |
| CRC10 | 10 | 26.9 |
| CRC15 | 20 | 42.8 |

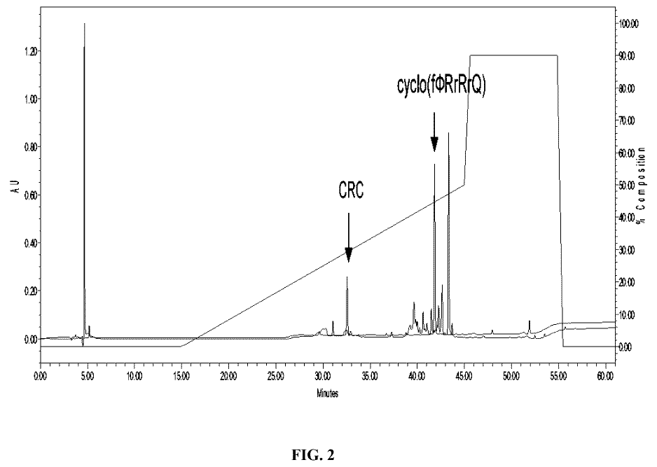

Polymer Characterization by Analytical HPLC. Analytical HPLC of the CRC5 polymer (from 5:1 CRC and CPP9) revealed a mixture of many species, with the most abundant species having a retention time of 43.3 min (FIG. 2). Treatment of the CRC5 polymer with 10 mM DTT converted the polymer into a mixture of predominantly two species, with retention times corresponding to those of CR&C and CPP9-miniPEG-Cys, suggesting that the CRC5 polymer was indeed formed, but contained a mixture of polymers of different numbers of CR₅C units.

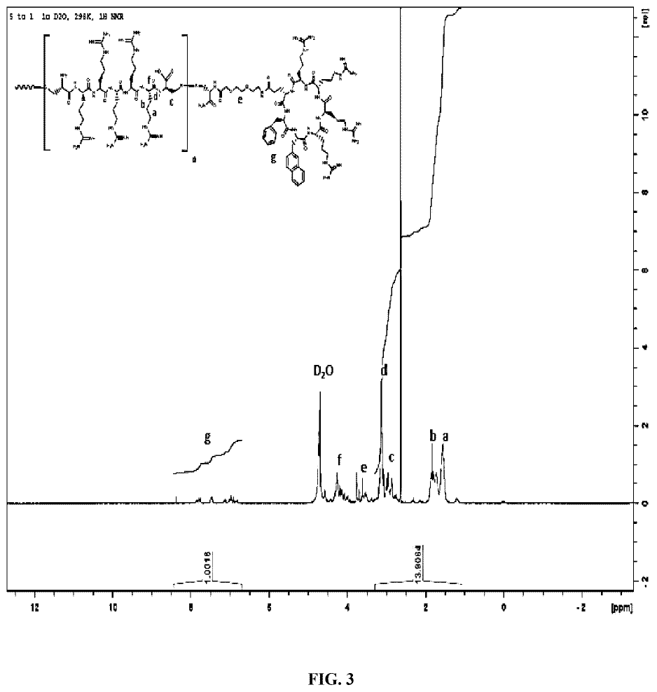

Estimation of MW by ¹H-NMR. The ¹H-NMR spectra of CRC5 and CRC10 polymers (5 mM concentration in 100% D₂O) were recorded at 600 MHz (Bruker, MA, USA). FIG. 3 shows the ¹H-NMR spectrum of CRC5. Resonances at δ1.4-1.6 (signal a), δ1.6-1.9 (signal b) and δ3.1-3.3 (signal d) were assigned to the —CH₂-groups in the arginine side chain. The signals at δ2.8-3.1 (signal c) were assigned to the —CH₂— group of cysteine. The signals at δ3.6-3.9 (signal e) were derived from the miniPEG linker. The signals at δ4.0-4.4 (signal f) were from the C—H's of amino acids. Signals at δ6.7-8.4 (g) are assigned to the aromatic protons from phenylalanine and naphthylalanine. The average number of repeating units (CR₅C) in the polymers (n) and the MW of the polymers were estimated by comparing the integrated signal intensities of the arginine side chains (signals a, b, c, d) and the aromatic protons (signal g). For the CRC5 polymer, n was found to be 8, corresponding to a MW of 10.6 kDa. For the CRC10 polymer, n was found to be 20 and the average MW was 22.6 kDa. These values are similar to the theoretical values (Table 5).

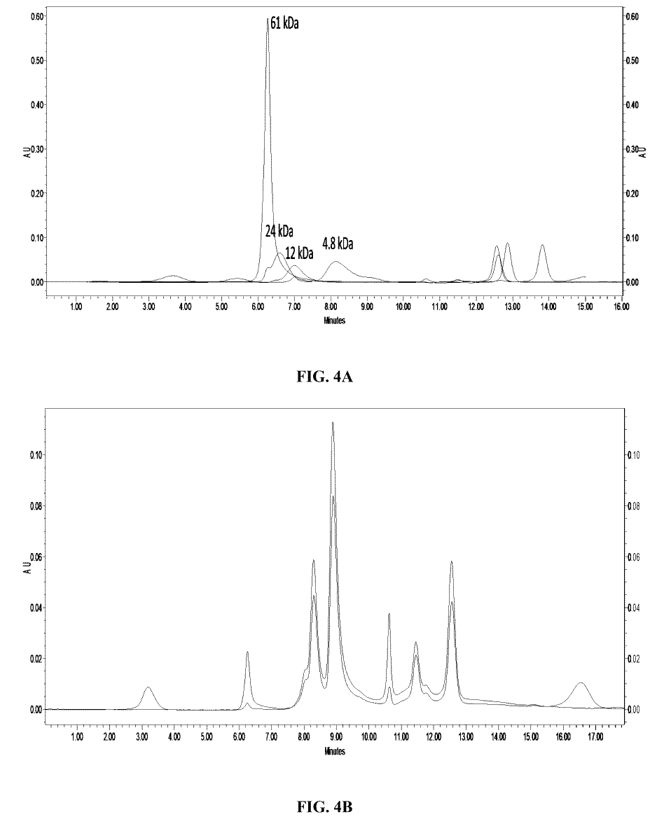

Estimation of MW by Gel-Filtration Chromatography (GPC). The MW distribution of the polymers (in 0.1 M NaCl) was assessed by GPC on a Waters 600 HPLC system (Waters, MA, USA) equipped with a TSKgel G3000PWx1-CP column (Tosoh Biosciences, CA, USA) and UV detection at 214 nm. Poly-L-lysine (PLL) of varying MWs (4.8, 12, 24 and 61 kDa) were used as MW standards (FIG. 4A). Comparison of the GPC chromatogram of CRC5 to PLL suggested that 1-2% of the reaction mixture corresponded to a species with MW˜60 kDa (peak at ˜6.2 min). The predominant species, however, had retention times of 8.3 and 9.0 min, corresponding to MW of 4-5 kDa and 2-4 kDa, respectively (FIG. 4B, top line at t=9 min). A similar MW distribution was observed for the CRC10 polymer. However, the ˜60 kDa species (peak at ˜6.2 min) was present at a higher percentage (˜15% of the total) than in CRC5 (FIG. 4B, bottom line at t=9 min). A species with very high MW (retention time=3.2 min) was also observed. It should be noted that neither the NMR method described above nor the GPC analysis can provide an accurate measurement of the MWs. More accurate MW determination will require prior separation of the polymer mixture into individual species and NMR analysis of the individual species.

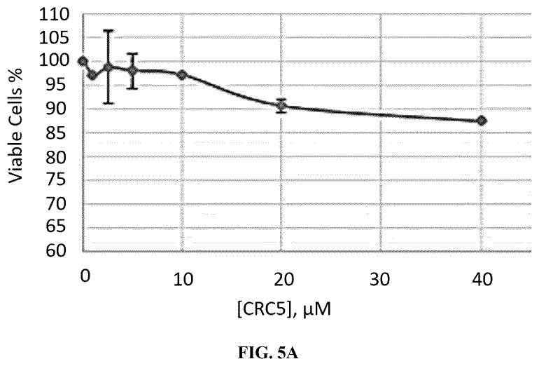

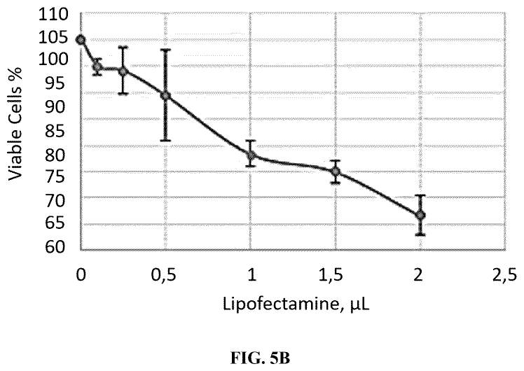

Cytotoxicity. The cytotoxicity of CRC5 polymer on HeLa cells was evaluated using the MTT assay. HeLa cells (3×10³ cells per well) in 100 μL of DMEM containing 10% FBS were seeded in 96-well plate and incubated for 24 h. Then cells were treated with varying concentrations (0-40 μM) of CRC5 and incubated at 37° C. with 5% CO₂ for 72 h. An MTT stock solution (10 μL; 5 mg/mL) was added into each well. The plate was incubated at 37° C. for 4 h. Then 100 μL of SDS-HCl solubilizing buffer was added into each well, and the resulting solution was mixed thoroughly. The plate was incubated at 37° C. overnight. The absorbance of the formazan product was measured at 570 nm on a Tecan M1000 plate reader. CRC5 was relatively non-toxic to HeLa cells, causing≤15% reduction in viability over the concentration range of 0 to 40 μM (or 0-500 μg/mL; FIG. 5A). In contrast, addition of 2 μL of the commercial lipofectamine solution (which is the recommended amount for transfection experiments) reduced the viability of HeLa cells by 35% (FIG. 5B).

siRNA Binding. The ability of CRC5 and CRC10 to bind siRNA targeting the firefly luciferase gene (siLuc) was tested by the gel retardation method using various N/P ratios, which correspond to the ratio between positively charged amino groups in the polymer (N stands for nitrogen in amino group) to negatively charged phosphate groups in the nucleic acid (P stands for phosphorous in phosphate group). The CRC polymer/siRNA complexes were formed with a fixed concentration of siRNA and increasing concentrations of CRC polymer to N/P ratios of 1:1 to 50:1. Free siRNA was used as a control. The samples were separated by electrophoresis on 1% agarose gel containing ethidium bromide at 70 V for 30 min in Tris-acetate-EDTA (TAE) buffer. While free siLuc RNA gave a discrete band on the gel, addition of the CRC polymers progressively decreased the intensity of the free RNA band and resulted in the formation of apparently an RNA/polymer complex that failed to migrate out of the sample loading well. Complete conversion of free siRNA into the RNA/polymer complex was observed at an N/P ratio of 10 for CRC5, while the corresponding ratio was 5 for CRC10. As expected, treatment of a preformed siLuc/CRC5 complex with 10 mM DTT resulted in a discrete band with the same mobility as the free RNA.

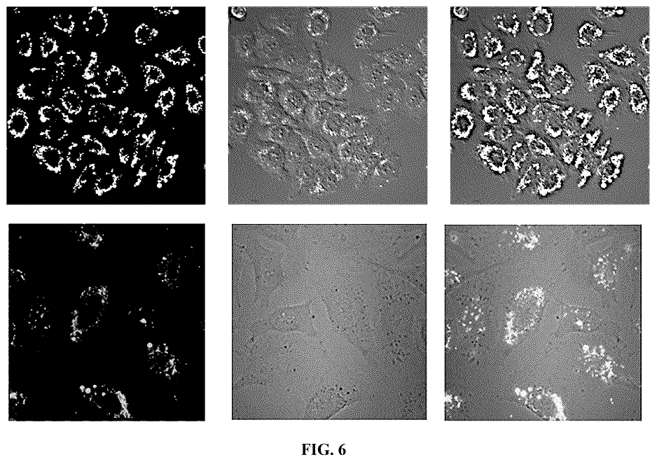

Cellular Uptake. To monitor the intracellular uptake of the CRC5/siLuc complex, 5′-fluorescein (FAM)-labelled siLuc (1 or 3 μM) was mixed with CRC5 to give an N/P ratio of 10 in OptiMEM or DMEM with 1% FBS and 1% Abs. HeLa cells were seeded in a 35 mm glass-bottomed microwell dish at a density of 3×10⁴ cells/mL and cultured overnight. Cells were washed twice with DPBS and treated with the CRC5/5′-FAM-siRNA complex for 2 h. After removal of the medium, the cells were gently washed with DPBS twice and imaged on a Nikon A1R live-cell confocal microscope equipped with a 100× oil objective or a Visitech Infinity 3 Hawk 2D-array live cell confocal microscope equipped with 60× oil objective. Data were analyzed using NISElemenets AR or MetaMorph Premier. A mix of diffuse and punctate fluorescence was observed inside the cytoplasm of all treated cells, indicating that the CRC5/siLuc complex was able to enter the cells and at least partially escaped from the endosomes into the cytosol (FIG. 6). In FIG. 6, live-cell confocal microscopic images of HeLa cells treated with the CRC5/5′-FAM-siLuc complex at 3 μM siRNA in OptiMEM (top) or at 1 μM siRNA in DMEM with 1% FBS, 1% Abs (bottom). Left, GFP channel; Center, DIC; and right, overlap of the above.

Knockdown of Luciferase Expression. The ability of CRC polymers to deliver siRNA intracellularly was tested on a HeLa cell line stably transfected with a firefly luciferase gene (HeLa-Luc). The siLuc sequences used were: sense 5′-CUUACGCUGAGUACUUCGAdTdT-3′ (SEQ ID NO: 137) and antisense 5′-UCGAAGUACUCAGCGUAAGdTdT-3′ (SEQ ID NO: 138). Hela-Luc cells were seeded onto 96-well plate at a density of 1.5×10⁴ cells/well in 100 μL of DMEM containing 10% FBS and cultured overnight. CRC5/siLuc complexes were formed by mixing CRC5 with siLuc in 20 mM HEPES, pH 7.4 followed by 15 min incubation. Complexes were then mixed with OptiMEM, added to cells and incubated at 37° C. for 24 or 48 h. Free siLuc, CRC5, Lipofectamine2000/siLuc, and pCRC/siLuc were used as controls. pCRC is a polymer of CRC without CPP9 at the two termini. Lipofectamine2000 was complexed with siRNA according to the manufacturer's protocol. A luciferase assay kit (OZ Biosciences) was used to quantitate the luciferase gene silencing level according to manufacturer's protocol. Luminescence was measured on a Tecan Infinite M1000 plate reader.

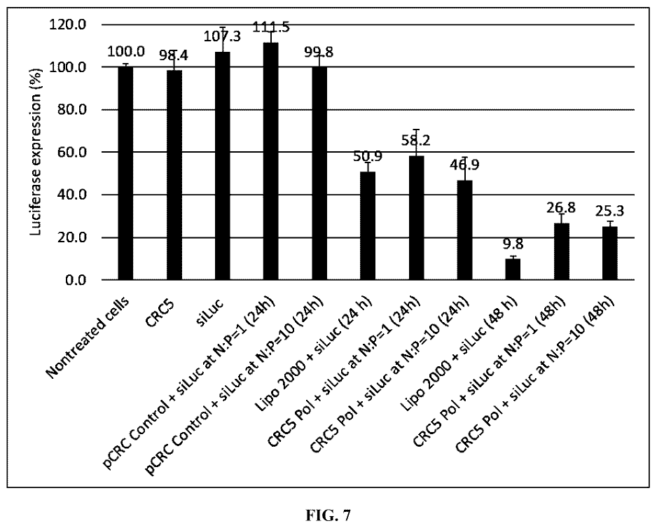

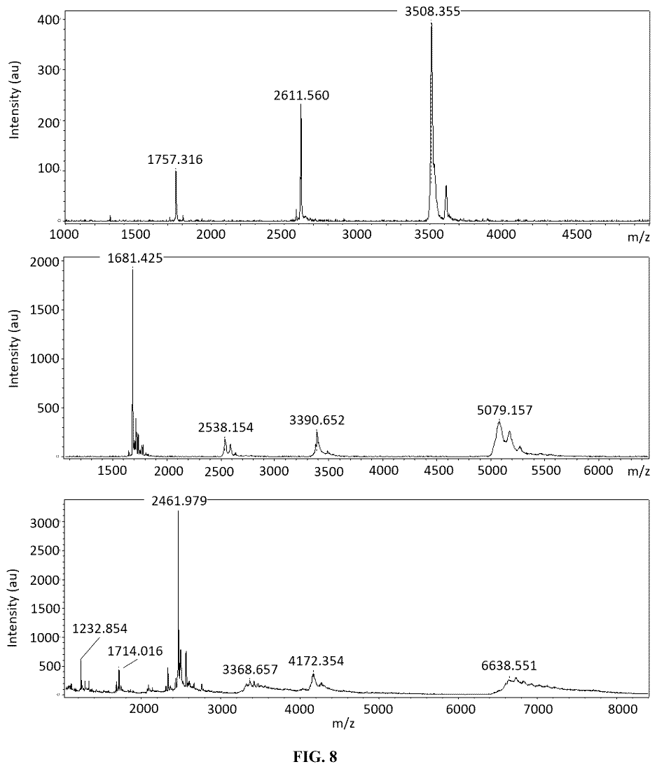

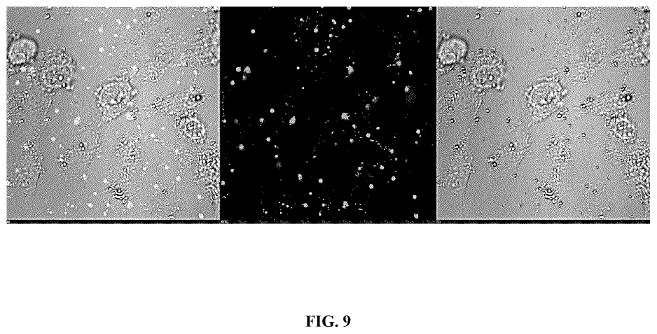

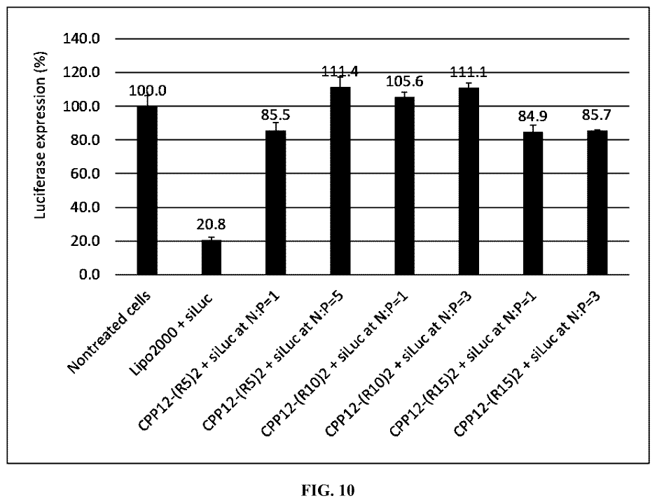

Treatment of HeLa-Luc cells with free siLuc, CRC5 alone, or the pCRC/siLuc complex did not reduce the luciferase expression (FIG. 9). On the other hand, the CRC5/siLuc complex substantially decreased the luciferase activity (50-75%) at all N/P ratios tested, with the highest silencing efficiency (75%) observed at an N/P ratio of 10 ([siLuc]=75 nM or 1 μg/mL and 6.6 μg/mL CRC5) and after 48 h of incubation. In fact, the gene silencing efficiency of the CRC5/siLuc complex was comparable to that of Lipofectamine2000/siLuc. However, as described above, CRC5 has much lower cytotoxicity than Lipofectamine. At their effective concentrations for siRNA delivery, lipofectamine 2000 (2 μL, the actual concentration of which is undisclosed by the commercial supplier) reduced the viability of HeLa-Luc cells by 35%, whereas CRC5 (6.6 μg/mL or ˜0.5 μM) showed no detectable cytotoxicity (FIG. 7).

### Example 2. Polypeptide Conjugate with One cCPP Terminal

Design and Synthesis. While the CRC polymers described in Example 1 are effective for siRNA delivery and simple to prepare, they are mixtures of different species, the precise MWs and structures of which can be more difficult to characterize.

In this example, a cyclic CPP was covalently linked to a 3,5-bis(mercaptomethyl)benzoyl (Bmb) moiety through a long, flexible linker, miniPEG-lysine (Scheme 1). CPP12, which has cytosolic delivery efficiency of 121%, was selected for this design (see Qian, 2016). Two polyarginine peptides (R5, R10, or R15) are conjugated to the Bmb scaffold through disulfide bonds. We envisioned that the two polyarginine peptides would bind tightly to double-stranded siRNA via electrostatic interactions, leaving the cyclic CPP exposed for cellular uptake. Upon entering the cytosol, the disulfide bonds would be cleaved by GSH, releasing the siRNA for biological function.

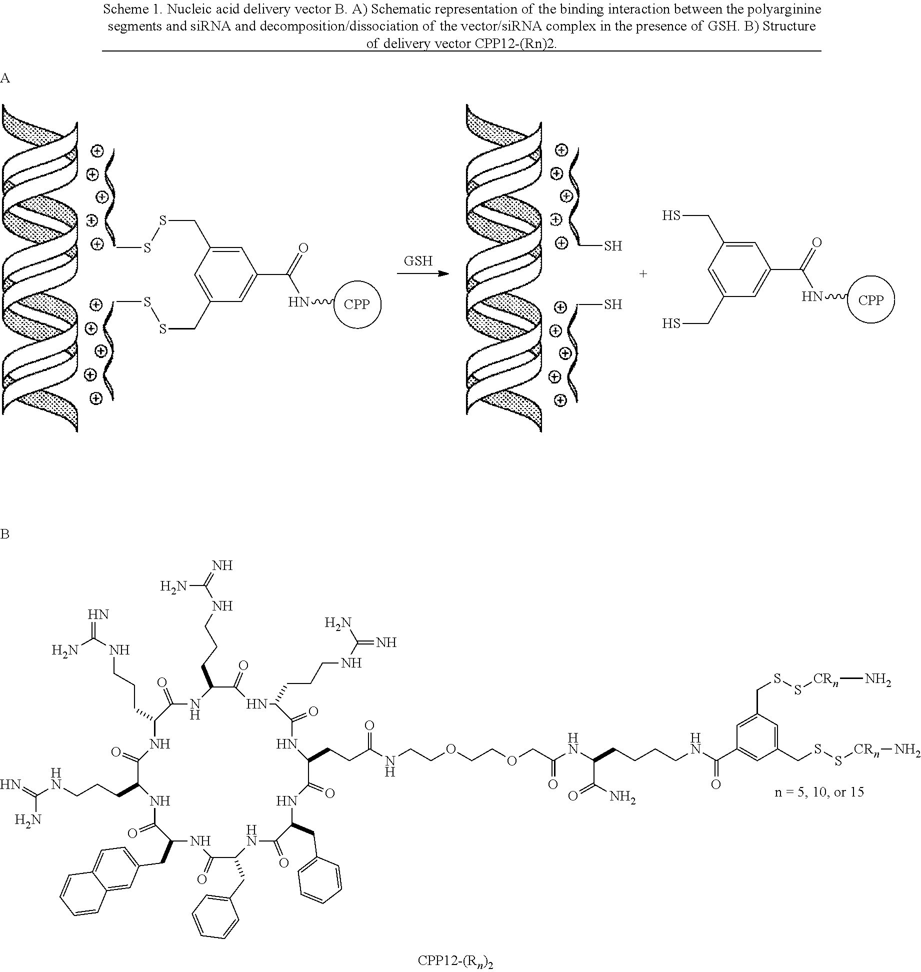

The cCPP-polyarginine conjugate was prepared by first synthesizing CPP12-miniPEG-Lys (Mtt)-NH₂ on Rink amide resin. The Mtt group on the lysine side chain was selectively removed by treatment with 2% TFA and bis(trityl)-protected Bmb was coupled to the lysine side chain with Oxyma/DIC/DIPEA as coupling reagents. The resulting peptide, CPP12-miniPEG-Lys (Bmb)-NH₂, was cleaved from the resin and side chain deprotected using the standard conditions and purified by reversed-phase HPLC. The freshly eluted peptide was treated with an excess of 2,2-dithiodipyridine to protect (and activate) the two thiol groups of Bmb. Meanwhile, the polyarginine peptides CRₙ, where n=5, 10 or 15, were synthesized on the solid phase with a cysteine residue added at the N-terminus. After deprotection, cleavage, and HPLC purification, the CRₙ peptide was mixed with the thiopyridylated CPP12 at neutral pH to afford the conjugates CPP12-(Rₙ)₂. The identity of the vectors was confirmed by MALDI-TOF mass spectrometry (Table 6, FIG. 8). In FIG. 8: MALDI-TOF MS spectra of CPP12-(R₅)₂ (top), CPP12-(R₁₀)₂ (middle), and CPP12-(R15)2 (bottom).

TABLE 6
MW of CPP12-(Rₙ)₂ polymers

| | MW | |
|---|---|---|
| Polymer | calculated | observed |
| CPP12-(R₅)₂ | 3510 | 3508 |
| CPP12-(R₁₀)₂ | 5076 | 5079 |
| CPP12-(R₁₅)₂ | 6636 | 6638 |

siRNA Binding. CPP12-(R₅)₂, CPP12-(R₁₀)₂ and CPP12-(R₁₅)₂ were tested for binding to siLuc by the gel retardation method using various N/P ratios as described above. The CPP12-(Rₙ)₂/siRNA complexes were formed with a fixed concentration of siRNA and increasing concentrations of CPP12-(Rₙ)₂ to give N/P ratios of 0.5:1 to 10:1. Free siRNA was used as a control. Complete conversion of free siRNA into the RNA/polymer complex was observed at an N/P ratio of 5 for CPP12-(R₅)₂ and N/P ratio of 3 for CPP12-(R₁₀)₂ and CPP12-(R₁₅)₂. Treatment of the preformed siLuc/CPP12-(R₅)₂ complex with 10 mM DTT resulted in a discrete band with the same mobility as the free RNA (FIG. 12, top right). On the other hand, similar treatment of the siLuc/CPP12-(R₁₀)₂ and siLuc/CPP12-(R₁₅)₂ complexes with 10 mM DTT did not result in a discrete RNA band (FIG. 12, middle and bottom right), indicating that R₁₀ and R₁₅ remain bound to siRNA after DTT treatment.

Cellular Uptake. 5′-FAM-labelled siLuc (3 μM) was mixed with CPP12-(R₅)₂ at an N/P ratio of 5 in OptiMEM medium. HeLa cells were treated with the CPP12-(R₅)₂/5′-FAM-siRNA complex for 2 h. Cells were washed and imaged by confocal microscopy as described above. Interestingly, CPP12-(R₅)₂ and 5′-FAM-siRNA formed large, insoluble particles of nanometer sizes (FIG. 9). In FIG. 9: center, GFP channel; right, DIC; and left, overlap of the above. Although some of the labeled siRNA was internalized by the cells, the intracellular fluorescence level was lower than that obtained with the copolymer of Example 1.

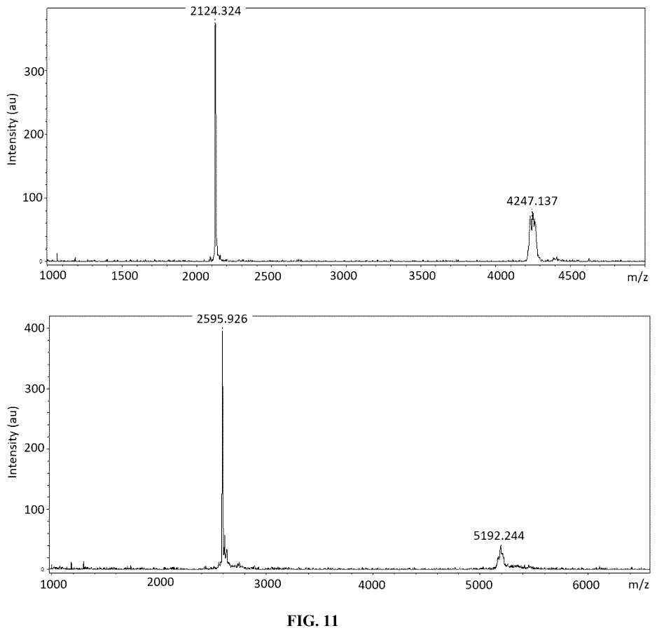

Knockdown of Luciferase Expression. The ability of CPP12-(Rₙ)₂ to deliver siLuc intracellularly was tested on HeLa-Luc cells as described above. CPP12-(Rₙ)₂/siLuc complexes were formed at various N:P ratios by mixing CPP12-(Rₙ)₂ with siLuc in 20 mM HEPES, pH 7.4 followed by 15 min incubation. The complexes were then mixed with OptiMEM medium, added to cells, and incubated at 37° C. for 48 h. Lipofectamine2000/siLuc was used as a positive control. As shown in FIG. 10, treatment of HeLa-Luc cells with the CPP12-(Rₙ)₂/siLuc complexes (75 nM siLuc) at N:P ratios of 1 to 5 only slightly reduced the expression of the luciferase (≤15%), in agreement with their inefficient cellular uptake (FIG. 9).

### Example 3. Modification of Peptide Conjugate in Example 2

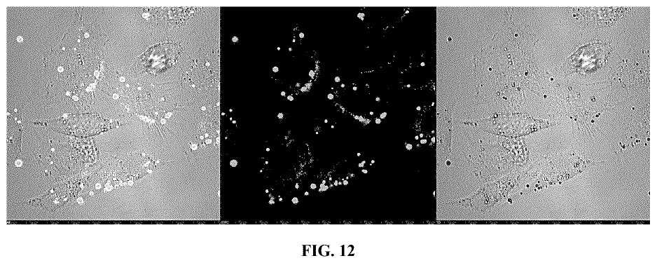

Design and Synthesis. In modifying the peptide conjugate of Example 2, cyclic CPP (e.g., CPP9) was directly attached to a polyarginine peptide (Rₙ) through a miniPEG linker. A cysteine was added to the C-terminus of the peptide. The entire molecule was readily synthesized by standard solid-phase peptide chemistry, cleaved off the solid support, deprotected, and purified by HPLC. Exposure of the peptide to an oxidant (e.g., DMSO) resulted in homodimerization through the formation of a disulfide between the C-terminal cysteines. Again, the (Rₙ)₂ moiety (where n=5 or 8) in the central section is expected to bind to siRNA with high affinity in the oxidizing extracellular environment, whereas the two terminal CPPs would mediate endocytic uptake. Once inside the cytosol, the disulfide bond would be, without bound to any theory, reduced and the siRNA would be released. One advantage of this design was thought to be that the presence of two CPPs should enhance the cellular uptake efficiency of the vector (relative to Example 2). The identity of the synthesized peptide conjugate was determined by MALDI-TOF mass spectrometry (Table 7 and FIG. 11). In FIG. 11, MALDI-TOF mass spectra of (CPP9-R₅)₂ (top) and (CPP9-R₈)₂ (bottom).

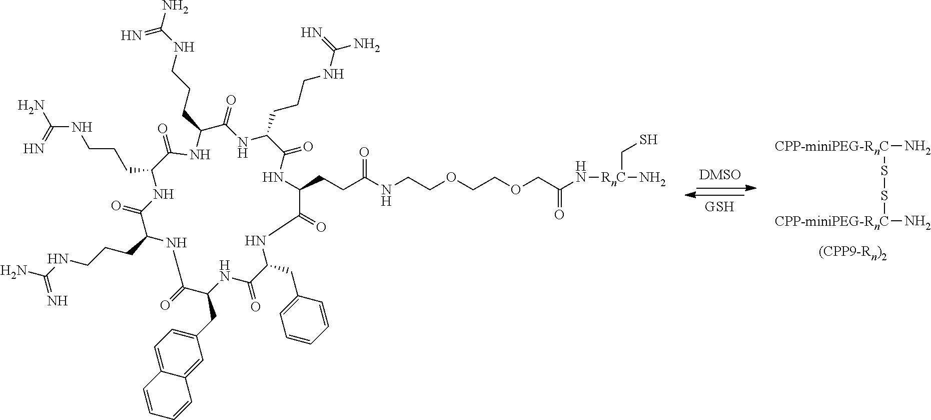

TABLE 7
MW of CPP9-Rₙ)₂ polymers

| | MW | |
|---|---|---|
| Polymer | calculated | observed |
| (CPP9-R₅)₂ | 4250 | 4247 |
| (CPP9-R₈)₂ | 5190 | 5192 |

siRNA Binding. (CPP9-R₅)₂ and (CPP9-R₈)₂ were evaluated for binding to siLuc by the gel retardation method at various N/P ratios as described above. The (CPP9-Rₙ)₂/siRNA complexes were formed with a fixed concentration of siRNA and increasing concentrations of CPP12-(Rₙ)₂ to give N/P ratios of 0.5:1 to 20:1. Free siRNA was used as a control. Complete conversion of free siRNA into the RNA/polymer complex was observed at an N/P ratio of 3 for (CPP9-R₅)₂ and N/P ratio of 1 for (CPP9-R₈)₂.

Cellular Entry by Confocal Microscopy. 5′-FAM-labelled siLuc (3 μM) was mixed with (CPP9-R₅)₂ at an N/P ratio of 2 in OptiMEM. HeLa cells were seeded and washed as described above and treated with the (CPP9-R₅)₂/5′-FAM-siRNA complex for 2 h. Cells were washed and imaged as described above. The complex formed insoluble aggregates which are clearly visible under the microscope. Internalization of the complex by HeLa cells was also evident and somewhat more efficient than the 1^{st}-generation vector (FIG. 12). In FIG. 12, Center, GFP channel; Right, DIC; and left, overlap of the above.

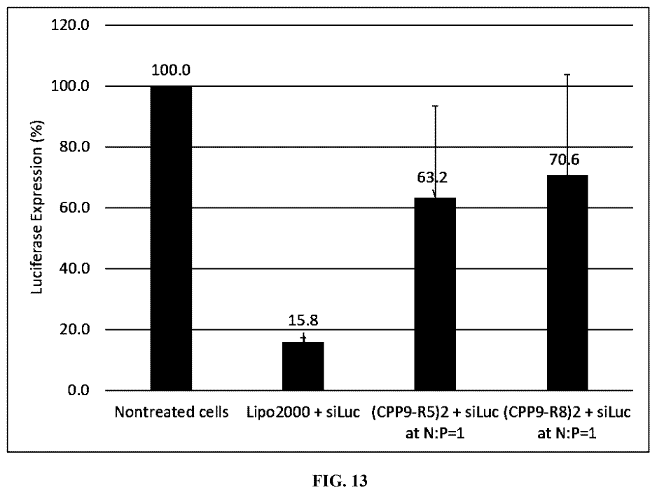

Knockdown of Luciferase Expression. The ability of (CPP9-Rₙ)₂ vectors to deliver siLuc intracellularly was tested on HeLa-Luc cells as described above. (CPP9-Rₙ)₂/siLuc complexes were formed at different N:P ratios by mixing (CPP9-Rₙ)₂ with siLuc in 20 mM HEPES, pH 7.4 followed by 15 min incubation. Complexes were then mixed with OptiMEM, added to the cells and incubated at 37° C. for 48 h. Lipofectamine2000/siLuc was used as a positive control. Treatment of HeLa-Luc cells with the (CPP9-R₅)₂/siLuc and (CPP9-R₈)₂/siLuc complexes at N:P ratio of 1:1 (75 nM siLuc) decreased the luciferase activity by 30-35% (FIG. 13). Interestingly, the (CPP9-R₈)₂/siLuc complex was slightly less effective than the (CPP9-R₅)₂/siLuc complex, likely because of the less efficient release of siRNA from the former.

### Example 4. Modification of Peptide Conjugate in Example 2

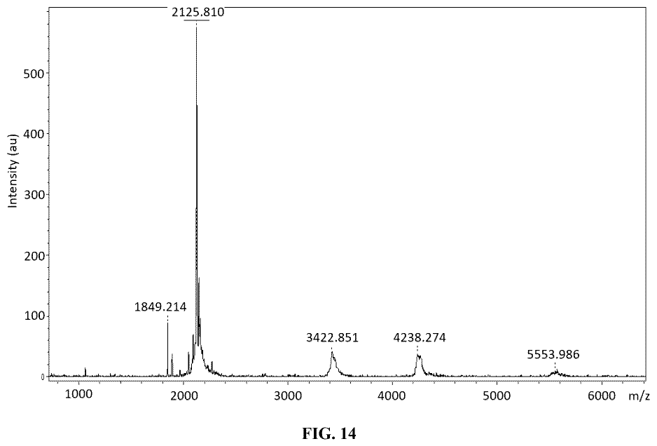

Design and Synthesis. The above Examples suggest that for optimal siRNA delivery efficiency, the polyarginine moiety should have sufficient length to bind to siRNA with high affinity and interact with entire length of the siRNA to prevent it from nuclease action. Once inside the cell, the polymer must be broken down into small fragments that readily dissociate from the siRNA. The polypeptide conjugate of this Example has the general structure of CPP9-R₅—S—S—R₆—S—S—R₅—CPP9 (SEQ ID NO: 144), which consists of two cyclic CPP9-miniPEG-R₅ (SEQ ID NO: 142) units covalently linked to an internal R₆ (SEQ ID NO: 143) unit through two disulfide bonds. The polypeptide conjugate of this Example is structurally very similar to the copolymers from Example 1, but can be readily synthesized as a single species. Briefly, the CPP9-miniPEG-R₅-Cys (SEQ ID NO: 145) unit was synthesized by standard solid-phase peptide chemistry and reacted with dithiodipyridine to give the thiopyrodylated form, which is stable upon storage. The internal R₆ unit has a Cys-β-Ala dipeptide on each side to give some flexibility to the polymer structure. Simply mixing a 2:1 ratio (mol/mol) of the CPP9-miniPEG-R₅ (SEQ ID NO: 142) and R₆ (SEQ ID NO: 143) units at the physiological pH gave the desired polymer as the predominant species, which was purified to homogeneity by reversed-phase HPLC. The identity of polypeptide conjugate of this Example was confirmed by MALDI-TOF mass spectrometry (FIG. 14).

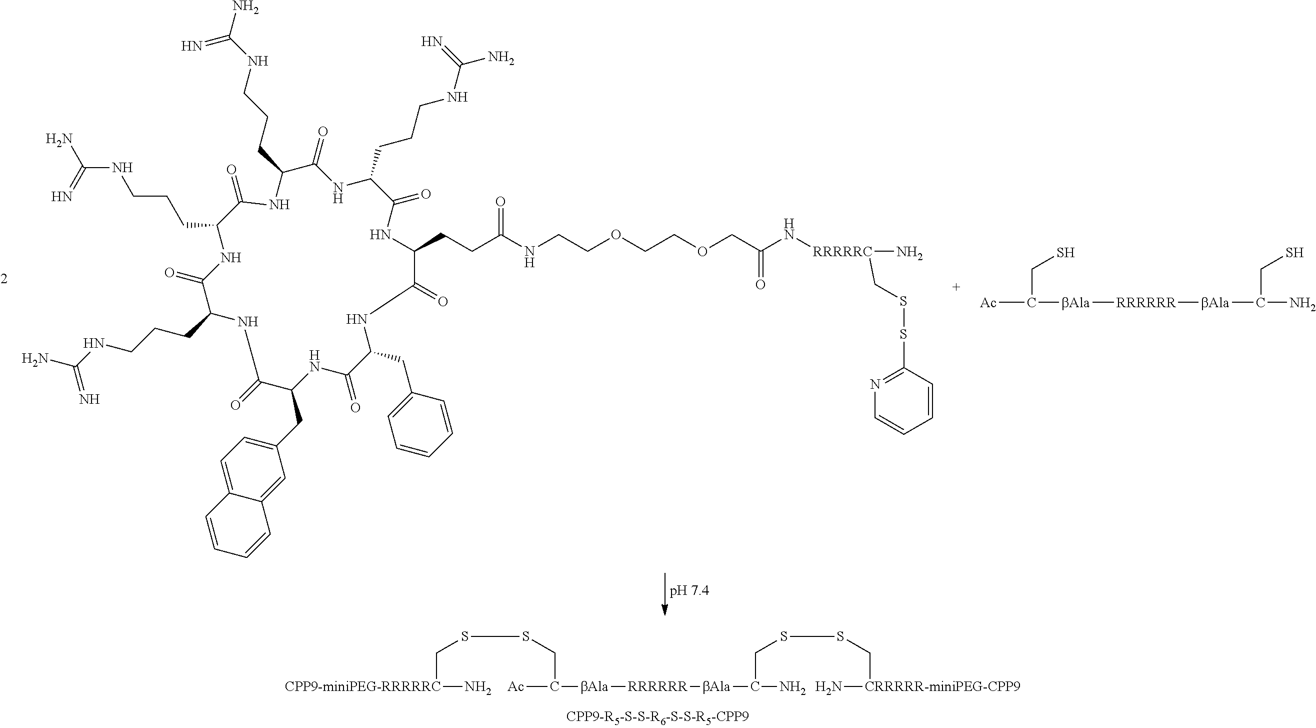

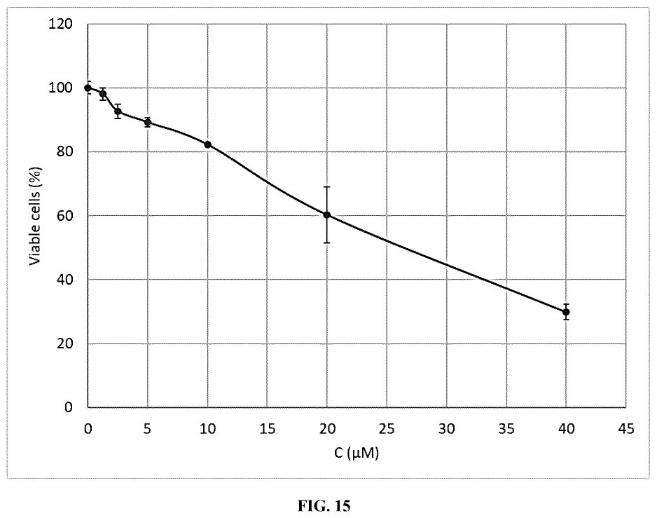

Cytotoxicity. The polypeptide conjugate of this Example was tested for cytotoxicity against HeLa cells using the MTT assay. HeLa cells were treated with varying concentrations (0-40 μM) of the 3rd-generation vector and incubated at 37° C. with 5% CO₂ for 72 h. Cells were treated with MTT stock solution and SDS-HCl solubilizing buffer as described above. The absorbance of the formazan product was measured at 570 nm on a Tecan M1000 plate reader. In contrast to the copolymers, which caused ≤20% reduction in viability up to 40 μM concentration (FIG. 5A), the polypeptide conjugate of this Example reduced the viability of HeLa cells in a dose-dependent manner, by 70% at 40 μM (FIG. 15).

siRNA Binding. The polypeptide conjugate of this Example was able to bind siLuc. Complete conversion of free siRNA into the siRNA/vector complex was observed at an N/P ratio of 2. Treatment of the preformed siLuc/vector complex with 10 mM DTT did not regenerate the free siRNA band; instead, a broad, smeared siRNA band was observed, indicating that siRNA remained bound to the polyarginine species even after reduction of the disulfide bonds.

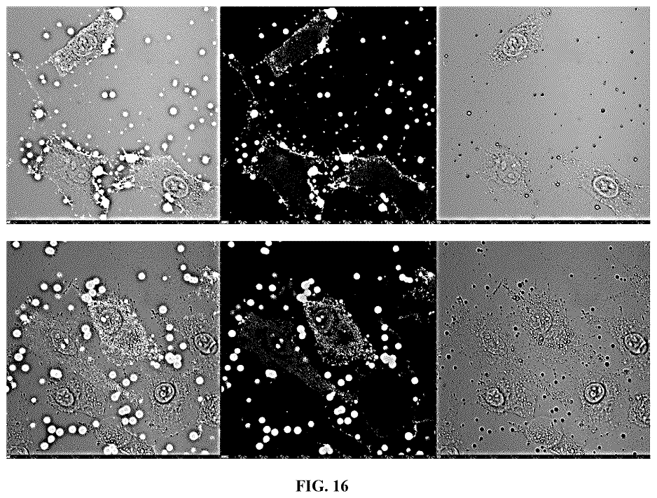

Cellular Uptake by Confocal Microscopy. 5′-FAM-labelled siLuc (1 or 3 μM) was mixed with the 3rd-generation vector at an N/P ratio of 2 in OptiMEM or DMEM with 1% FBS. HeLa cells were treated with the polypeptide conjugate of this Example/5′-FAM-siRNA complex for 2 h. Cells were washed and imaged by live-cell confocal microscopy. Cellular entry of the polypeptide conjugate of this Example/5′-FAM-siRNA complex was more efficient than the Examples 2 or 3, and the diffuse fluorescence throughout the entire cell volume indicate that the complex had escaped from the endosome into the cytosol and nucleus (FIG. 16). Formation of insoluble aggregates was still observed. In FIG. 16, at 1 μM siRNA in DMEM with 1% FBS (top) or 3 μM siRNA in OptiMEM (bottom). Center, GFP channel; Right, DIC; and left, overlap of the above.

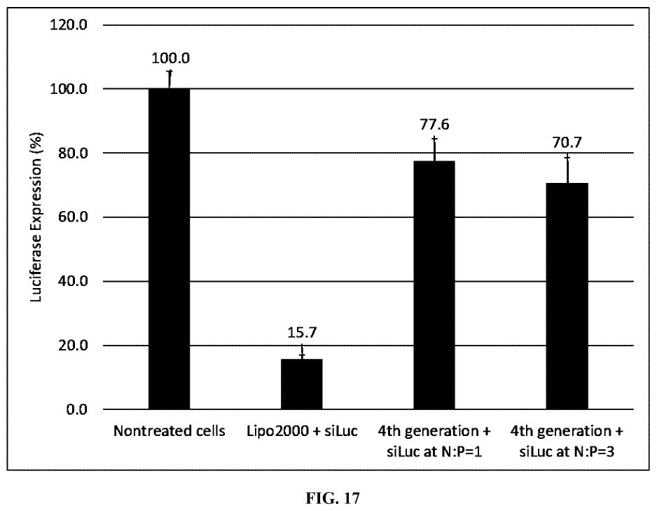

Knockdown of Luciferase Expression. Treatment of HeLa-Luc cells with the 3 polypeptide conjugate of this Example/siLuc complexes at N:P ratios of 1:1 and 3:1 (both at 75 nM siLuc RNA) for 48 h at 37° C. reduced the luciferase expression by 22% and 29%, respectively. Under the same conditions, the lipofectamine2000/siLuc complex reduced the luciferase expression by 84% (FIG. 17).

Knockdown of Firefly Luciferase Expression Normalized by Renilla Luciferase Expression. The ability of CRC polymers to deliver siRNA intracellularly was tested on a HeLa cell line stably transfected with Firefly and Renilla luciferase genes (Dual-HeLa). Firefly luciferase serves as an experimental reporter, while Renilla luciferase serves as a control reporter allowing to normalize the specific gene silencing of an experimental reporter. The siLuc sequences used were: sense 5′-AAmCGmCmUGGGmCGmUmUAAmUmCAAdTdT-3′ (SEQ ID NO: 139) and antisense 5′-UUGAUmUAACGCCmCAGCGUUdTdT-3′ (SEQ ID NO: 140). Dual-HeLa cells were seeded onto white 96-well plates at a density of 1.0×104 cells/well in 100 μL of DMEM containing 10% FBS and cultured overnight. CRC5/siLuc complexes were formed by mixing CRC5 with siLuc in 20 mM HEPES, pH 7.4 followed by 15 min incubation. Complexes were then mixed with OptiMEM, added to cells and incubated at 37° C. for 48 h. Lipofectamine 2000/siLuc was used as a positive control. Lipofectamine 2000 was complexed with siRNA according to the manufacturer's protocol. Dual-Glo Luciferase Assay System (Promega, USA) was used to quantitate the luciferase gene silencing level according to manufacturer's protocol. Luminescence was measured on a Tecan Infinite M1000 plate reader.

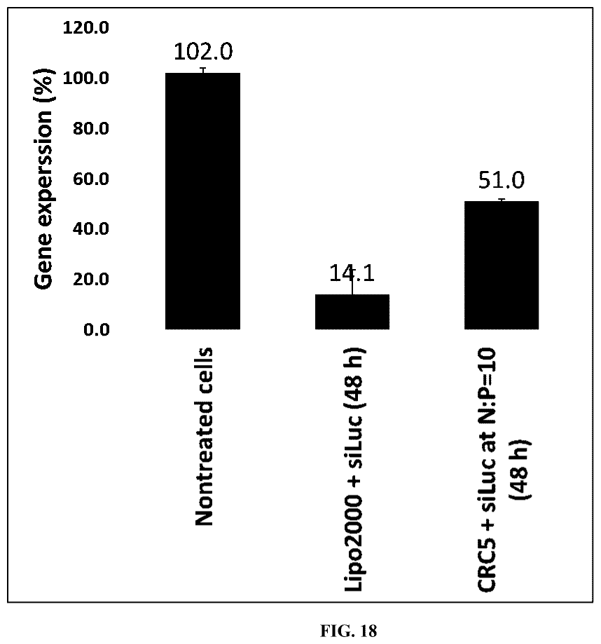

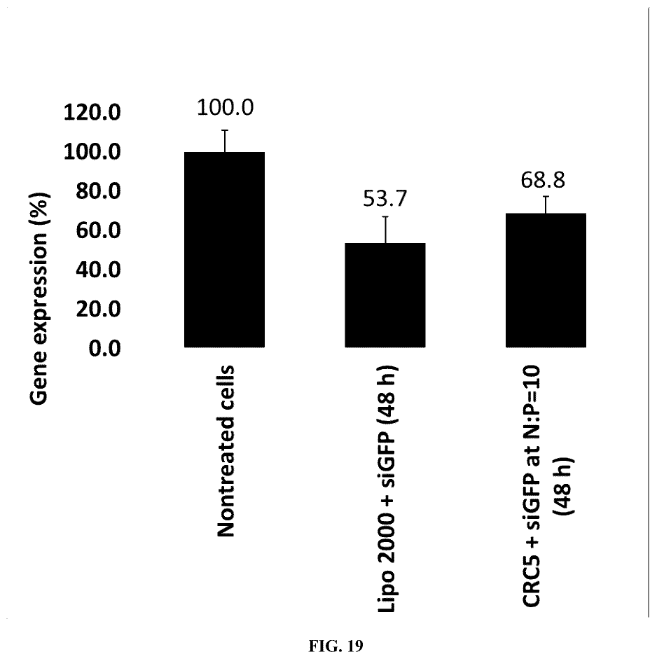

Treatment of HeLa-Luc cells with the CRC5/siLuc complex decreased the Firefly luciferase activity by 49% at an N:P ratio=10 ([siLuc]=75 nM and 6.6 μg/mL CRC5) after 48 h of incubation after normalization by Renilla luciferase expression (FIG. 18). Under similar conditions, Lipofactamine 2000 resulted in 86% reduction.

Knockdown of GFP Expression. The ability of CRC polymers to deliver siRNA intracellularly was also tested on a HeLa cell line stably transfected with GFP gene (HeLa-GFP). The siGFP used in this experiment was Silencer™ GFP (eGFP) siRNA (#AM4626, Thermo Fisher, USA). HeLa-GFP cells were seeded onto 96-well plate at a density of 1.0×10+ cells/well in 100 μL of DMEM containing 10% FBS and cultured overnight. CRC5/siLuc complexes were formed by mixing CRC5 with siLuc in 20 mM HEPES, pH 7.4 at N:P=10 followed by 15 min incubation. Complexes were then mixed with OptiMEM, added to cells and incubated at 37° C. for 48 h. Lipofectamine 2000/siLuc was used as a positive control. After incubation cells were lysed on ice for 30 min in IP lysis buffer supplemented with protease and phosphatase inhibitors. Cell lysates were centrifuged and 15000 rpm for 10 min, and the extracted proteins were collected. Protein concentration was measured using a BCA Protein Assay Kit (Thermo Fisher, USA) and equal amount of total proteins (˜200 ng) were added to a black 384-well plate. Fluorescence intensity was measured at 510 nm on a Tecan Infinite M1000 plate reader.

Treatment of HeLa-GFP cells with the CRC5/siGFP complex at N:P ratio=10 ([siLuc]=75 nM and 6.6 g/mL CRC5) decreased the GFP expression by 31% after 48 h of incubation, which was comparable to the efficiency of Lipofectamine 2000 (46%; FIG. 19).

### Discussion (Examples 1-4)

Without bound to any theory, the ideal siRNA delivery vector (polypeptide conjugates) should bind to siRNA with high affinity and interact with the entire siRNA molecule to prevent the latter from nuclease action. Once inside the cell, however, the vector should be readily broken down into small fragments that readily dissociate from the siRNA. In addition, because liposome- and nanoparticle-based siRNA delivery systems often resulted in accumulation of siRNA in well-vascularized tissues such as the liver, spleen, and kidney, soluble, oligomeric vector/siRNA complexes (ideally 1:1 vector/siRNA complex) which should have better tissue penetration and potentially broader distribution in vivo are desirable. All four siRNA delivery vectors described in the Examples are capable of delivering siRNA into mammalian cells and knocking down the expression of luciferase gene, but meet the above design criteria to different extents. Overall, the polypeptide of Example 1 demonstrated better performance than Examples 2-3. It very effectively delivers siRNA into the cytosol of mammalian cells (as evidenced by confocal microscopy) and most efficiently knocks down luciferase expression. It exhibited minimal cytotoxicity to HeLa cells at up to 40 μM concentration. It did not form any insoluble species (i.e., nanoparticles). It is also operationally very simple to prepare. The challenge with Example 1's polypeptide conjugate may be its structural heterogeneity (i.e., different number of R₅ units in the copolymer), which may require extra attention during their preparation in order to produce copolymers of consistent compositions.

The three vectors as prepared according to Examples 2-4, on the other hand, have well-defined structures (single species) and are readily prepared in pure forms. However, the challenge was the formation of insoluble aggregates and generally having lower siRNA delivery efficiencies and higher cytotoxicities. Presumably, without bound to any theory, their smaller sizes (compared to the conjugate of Example 1) result in weaker binding to siRNA when outside the cell and during endocytic uptake. Because their polyarginine sections may be too short to cover the entire siRNA surface, the cCPPs at the termini may bind to the siRNA as well, potentially interfering with their membrane binding and CPP function. At the same time, binding of multiple polyarginine peptides to one siRNA molecule (and/or multiple siRNA molecules to the same polyarginine peptide) could condense nucleic acids into large, insoluble nanoparticles, as was commonly observed for other arginine-rich CPPs such as Tat (see Arthanari, Y. et al., J. Controlled Release 2010, 145, 272-280) and R9 (see Law, M. et al., Biotechnol Prog, 2008, 24, 957-963). After cytosolic entry and reduction of the disulfide bonds, the fragments derived from the vectors in Examples 2-4 contain 9 or more arginine residues [except for CPP9-(R₅)₂]. As demonstrated by gel electrophoresis in Examples 2 and 4, these fragments remain tightly associated with the siRNA and reduce the knockdown efficiency of the siRNA. Additionally, the polyarginine fragments may bind to endogenous nucleic acids inside the cell, causing cytotoxicity. In comparison, reduction of the copolymer of Example 1 produces fragments with 5 or less arginine residues, which without bound to any theory do not bind to siRNA or other nucleic acids with high affinity as demonstrated by gel electrophoresis in Examples 1.

The Examples demonstrate that the copolymerization of cCPPs and pentaarginine peptides through disulfide bonding generated a family of cationic copolymers that bind siRNA with high affinity, effectively deliver them into the cytosol of mammalian cells, and then release them for functional knockdown of specific mRNA levels. This novel delivery method would be useful in delivering nucleic acids to target cells.

The publications discussed herein are provided solely for their disclosure prior to the filing date of the present application. Nothing herein is to be construed as an admission that the present invention is not entitled to antedate such publication by virtue of prior invention.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

We claim:

1. A compound of Formula: 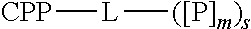
or a pharmaceutically acceptable salt thereof;
wherein:
P is a polypeptide comprising at least three monomers selected from arginine, lysine, and histidine;
m and s are 1;
L is a Linker comprising a —(CH₂CH₂—O)_{z}— moiety wherein z is an integer between 1 and 20;
CPP is a cyclic polypeptide conjugate;
wherein the compound also comprises a cargo moiety.

2. The compound of claim 1, wherein P comprises at least three lysine monomers.

3. The compound of claim 2, wherein the cargo is an antisense oligonucleotide.

4. The compound of claim 3, wherein CPP comprises between 4 and 14 amino acid monomers.

5. The compound of claim 3, wherein CPP comprises 8 amino acid monomers.

6. The compound of claim 5, wherein P comprises at least one arginine monomer.

7. The compound of claim 1, wherein CPP is capable of penetrating a cell membrane.

8. The compound of claim 1, wherein CPP comprises 7 amino acid monomers.

9. The compound of claim 1, wherein CPP comprises 8 amino acid monomers.

10. The compound of claim 1, wherein CPP comprises 9 amino acid monomers.

11. The compound of claim 1, wherein each amino acid of CPP is a natural amino acid.

12. The compound of claim 11, wherein CPP comprises 8 amino acid monomers.

13. The compound of claim 12, wherein P comprises at least three lysine monomers.

14. The compound of claim 1, wherein CPP comprises at least two arginines and at least two hydrophobic amino acids.

15. The compound of claim 1, wherein the cargo is a therapeutically active agent.

16. The compound of claim 1, wherein the cargo is an antisense oligonucleotide.

17. The compound of claim 1, wherein CPP is of Formula: 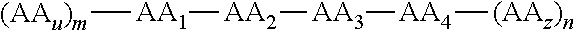
and wherein:
each of AA₁, AA₂, AA₃, and AA₄, are independently selected from a D or L amino acid or glycine;
each of AAᵤ and AA_{z} are independently selected from a D or L amino acid or glycine; and
m is 1 and n 0, 1, 2, 3, 4, 5, or 6.

18. The compound of claim 17, wherein at least two of AA₁, AA₂, AA₃, AA₄, AAᵤ, and AA_{z} are independently arginine.

19. The compound of claim 18, wherein at least two of AA₁, AA₂, AA₃, AA₄, AAᵤ, and AA_{z} are independently an amino acid with a hydrophobic side chain.

20. The compound of claim 19, wherein at least one amino acid with a hydrophobic side chain is glycine.

21. The compound of claim 20, wherein an arginine is adjacent to an amino acid with a hydrophobic side chain.

22. The compound of claim 20, wherein an arginine is adjacent to a glycine.

23. The compound of claim 20, wherein two arginines are adjacent to glycines.

24. The compound of claim 19, wherein at least one amino acid with a hydrophobic side chain is phenylalanine.

25. The compound of claim 24, wherein an arginine is adjacent to an amino acid with a hydrophobic side chain.

26. The compound of claim 24, wherein an arginine is adjacent to a glycine.

27. The compound of claim 24, wherein two arginines are adjacent to glycines.
